(19) United States
(12) Patent Application Publication
(10) Pub. No.: US 2003/0134353 A1
(43) Pub. Date: Jul. 17, 2003

(54) Recombinant dimorphic fungal cell

(75) Inventors: Anne Mette Wolff, Gentofte (DK); Karen Fuglede Appel, Copenhagen (DK); Jesper Breum Petersen, Vanlose (DK); Ulla Poulsen, Virum (DK); Jose Arnau, Hellerup (DK); Mette Dorph Jacobsen, Lyngby (DK)

Correspondence Address: BROWDY AND NEIMARK, P.L.L.C., 624 NINTH STREET, NW, SUITE 300, WASHINGTON, DC, 20001-5303, US

(73) Assignee: Bioteknologisk Institut, Hosholm (DK)

(21) Appl. No.: 10/092,947

(22) Filed: Mar. 8, 2002

Related U.S. Application Data
(60) Provisional application No. 60/274,650, filed on Mar. 12, 2001.

(30) Foreign Application Priority Data
Mar. 8, 2001 (DK) PA 2001 00395

(51) Int. Cl. C12P 21/02; C12N 1/16; C07H 21/04

(52) U.S. Cl. 435/69.1; 435/254.8; 435/320.1; 530/350; 536/23.1

(57) ABSTRACT

It is an object of the present invention to provide fungal host organisms capable of expressing recombinant proteins while at the same time exhibiting satisfactory growth characteristics. It is a further object to provide—in a single fungal host organism—the characteristic of homogeneous growth and low viscosity typically associated with yeast organisms, and the capability for high protein secretion normally associated with filamentous fungi. It is a yet further object of the invention to provide useful tools for genetic analysis in zygomycetes, including dimorphic zygomycetes. Accordingly, the present invention relates to a recombinant, fungal cell or dimorphic fungal cell comprising a regulatable expression of a regulator of morphology. The nucleotide sequence encoding the at least one regulator of morphology is operably linked to an expression signal not natively associated therewith. Expression of the at least one regulator of morphology directed by the expression signal not natively associated therewith results in a dimorphic shift of a dimorphic fungal cell or a desirable, improved filamentation of a fungal cell or a dimorphic fungal cell. The improved filamentation of the fungal cell or the dimorphic fungal cell is positively correlated with an increased production and/or secretion of a desirable polypeptide.

This application is a nonprovisional of U.S. provisional application Serial No. 60/274,650 filed on Mar. 12, 2001, which is hereby incorporated by reference in its entirety. All patent and nonpatent references cited in the application, or in the present application, are also hereby incorporated by reference in their entirety.

### TECHNICAL FIELD OF THE INVENTION

The present invention relates to a recombinant, dimorphic fungal cell, including a cell comprising a regulatable expression of a regulator of morphology. The nucleotide sequence encoding the regulator of morphology is operably linked to an expression signal not natively associated therewith. Regulated expression of the regulator of morphology directed by the expression signal not natively associated therewith results in a dimorphic shift and/or an improved filamentation of the dimorphic fungal cell. The improved filamentation of the dimorphic fungal cell is positively correlated with an increased production and/or secretion of a desirable polypeptide.

It is an object of the present invention to provide fungal host organisms capable of expressing recombinant proteins while at the same time exhibiting satisfactory growth characteristics. It is a further object to provide—in a single fungal host organism—the characteristic of homogeneous growth and low viscosity typically associated with yeast organisms, and the capability for high protein secretion normally associated with filamentous fungi. It is a further object of the invention to provide useful tools for genetic analysis in zygomycetes, including dimorphic zygomycetes.

### BACKGROUND OF THE INVENTION

The use of recombinant host cells in the expression of heterologous proteins has in recent years greatly simplified the production of large quantities of commercially valuable proteins, which otherwise are obtainable only by purification from their native sources. Currently, different expression systems including prokaryotic and eukaryotic hosts are available. The selection of an appropriate expression system will often depend on the ability of the host cell to produce adequate yields of the protein as well as on the intended end use of the protein and the requirement for post-transcriptional modifications (e.g., glycosylation, etc).

Although mammalian cells and yeasts are the most commonly used eukaryotic hosts, filamentous fungi may possibly also be used as host cells for recombinant protein production. However, the use of filamentous fungi for recombinant protein production is often associated with several practical problems.

Filamentous fungi are recognized for their ability to produce and secrete high amounts of proteins, and certain species of e.g. the genus Aspergillus have been used effectively as host cells for recombinant protein production. Although Aspergillus niger and Aspergillus oryzae are routinely used in recombinant protein production, several notable drawbacks are associated with using such strains.

In particular, the morphology of filamentous fungi is not optimal for growth in fermentors, as the viscosity of the culture tends to become rather high as biomass increases. Increased viscosity limits the ability to mix and aerate the fermentation culture, and this leads to oxygen and nutrient starvation of the mycelia, which therefore becomes non-viable and unproductive.

Furthermore, filamentous growth in a fermentor is often associated with the formation of an uneven distribution of too dense aggregates of mycelium. This also results in nutrient starvation.

Accordingly, for commercial purposes, there continues to be a need for fungal host organisms capable of expressing recombinant proteins while at the same time exhibiting satisfactory growth characteristics. In particularly, there is a need for combining—in a single fungal host organism—the characteristic of homogeneous growth and low viscosity typically associated with yeast organisms, and the capability for high protein secretion normally associated with filamentous fungi.

Many members of the fungal kingdom have a distinguishing feature, dimorphism, which is the ability to switch between two morphological forms: a yeast form and a filamentous form. The morphological transition between yeast and filamentous forms is rather variable and encompasses pseudohyphal and true filamentous growth.

Some species display pseudohyphal but also true filamentation (e.g., Candida). Yet another class of dimorphism occurs when differentiated hyphae disarticulate to generate young cells, called arthroconidia or thalloconidia (e.g., Geotrichum, Arxula; Wartmann et al., 1995).

The morphological state of these organisms is determined by a combination of environmental stimuli and is often associated with pathogenesis (Madhani and Fink 1998). The yeast stage is normally unicellular and uninucleated, while the filamentous phase is multicellular. A remarkable exception to this rule is found in fungi belonging to the Zygomycetes (e.g., Mucor). In Mucor, both filamentous and yeast growth is organized in a single multinucleated cell. Mucor yeasts are multipolar (daughter cells can originate at different positions of the mother cell as opposed to bipolar and monopolar) each harboring more than one nucleus, while mycelium is aseptate but with evenly distributed nuclei.

Although Mucor species display a variety of differentiated hyphal morphologies, mainly associated with arthrospores, sporangiospores, or zygospores, only those species capable of growing in the form of spherical, multipolar, budding yeasts are referred to as dimorphic. Examples of dimorphic Mucor species include M. circinelloides, M. rouxii, M. genevensis, M. bacilliformis, and certain strains of M. subtilissimus.

M. circinelloides and M. racemosus are used interchangably in the art. For example, M. racemosus R7B has two different names in the ATCC collection, M. racemosus and M. circinelloides, and any mentioning herein of M. circinelloides shall be understood to refer to M. circinelloides (syn. racemosus), thus covering also strains designated as M. racemosus in the art. The bibliography of strain R7B (ATCC 90680) and the parental strain ATCC 1216b shows that American groups have traditionally called the organism as M. racemosus, while European groups have always used M. circinelloides.

Species of Mucor genetically constrained to a monomorphic existence include M. mucedo, M. hiemalis, M. miehei, M. pusillus, M. rammanianus, and certain strains of M. subtilissimus. Besides Mucor, only two other genera of dimorphic zygomycetes have been described. These are Mycotypha and Cokeromyces, both of which within the order Mucorales.

Mucor species are capable of generating three different types of spores. Zygospore are spiny, black, thick-walled structures. Germination of zygospores result in the formation of sporangiospores. Sporangiospores are formed only on a solid substractum under an aerobic atmosphere. Mucor sporangiospores are characteristically ellipsoidal in shape.

The sporangiospore is capable of developing into either the yeast or hyphal form upon germination, the precise morphological direction taken being dependent, among other things, on the nutritional and gaseous environments. This capability establishes Mucor species as unique among commonly studied microbial models of development in that they are faced with a bifurcation in the morphogenetic sequence at which critical regulatory responses presumably direct the organism to construct one or the other of alternative morphologies. Irrespective of whether the sporangiospore ultimately develops into a budding yeast cell or a hyphal germ tube, it is initially likely to undergo a period of spherical growth.

Arthrospores represent the least studied and most poorly understood cell type made by Mucor species. Although arthrospores derive only from hyphae, arthrospores from dimorphic species of Mucor also have the ability to germinate into either yeasts or hyphae depending on their environment. Such morphogenetic conversions have not yet been studied or described in any detail.

Accordingly, Mucor hyphae may develop from any of the spore types mentioned above and from Mucor yeasts, and Mucor yeasts may develop from sporangiospores, arthrospores, and hyphae of dimorphic species.

The initial phase of yeast development from sporangiospores is indistinguishable from that described above for hyphal development from sporangiospores. It should be noted that hyphal fragments persist as a significant portion of the cell population for a considerable time after the initiation of hypha-to-yeast morphogenesis. The resulting, mixed-cell population has so far dissuaded researchers from studying morphological conversions in this direction in any significant detail.

Much of the knowledge about the molecular mechanisms underlying the dimorphic switch has been obtained from studies of yeast and pseudohyphal differentiation in Saccharomyces cerevisiae. Different signal transduction pathways are involved in regulating the transition between these two forms in the budding yeast S. cerevisiae (Roberts and Fink 1994; FIG. 2), and evidence is now emerging that homologous signaling modules are involved in regulating filament formation in a range of other fungi. In S. cerevisiae and C. albicans, parallel signal transduction pathways are involved in dimorphism, namely a cAMP dependent and a MAP kinase-dependent pathway (FIG. 2). However, large differences in the stimuli, regulation and control of the dimorphic shift are found in different fungi. As an example, exogenous addition of cAMP to Candida albicans (Ernst 2000) promotes filamentation whereas in Mucor circinelloides results in ‘constitutive’ yeast growth (Orlowski 1991). Thus, different role and control of the cAMP dependent protein kinase A in these two fungi must exist.

Biochemical data on Mucor dimorphism have been extensively reviewed (Orlowski 1991). Generally, anaerobiosis and the presence of a fermentable hexose result in yeast growth, while aerobiosis and nutrient limitation are associated with filamentous growth. However, a gradient in the requirements for yeast or filamentous growth is found within the genus Mucor. M. genevensis can grow as yeast in aerobiosis if supplied with a high concentration of hexose; M. rouxii requires both hexose and anaerobic conditions to grow as a yeast and M. circinelloides cannot grow as yeast anaerobically at all unless an hexose is present in the medium.

In addition to morphopoietic agents, oxygen, CO₂, and hexoses occuring naturally in the external environment, a variety of synthetic compounds have also been reported to alter Mucor cell morphology. Some, but not all of these, have been useful when studying the regulation of Mucor morphogenesis.

Substances that inhibit mitochondrial energy-generating functions lock at least some Mucor species into the yeast form under aerobic conditions. This prevents the generation of an increased filamentation and/or prevents a dimorphic shift from yeast to filamentous fungal cell.

Also, certain morphopoietic agents with the capacity to induce aerobic growth in the yeast form do so only if a hexose is present in the medium. It has also been reported that e.g. an elevated level of fermentative metabolism can be observed in cells chemically induced to grow as yeasts under aerobic conditions. This observation suggests a linkage between alcoholic fermentation and yeast morphology, but the issue remains largely unresolved.

Although a given set of environmental parameters and conditions can generally be expected to evoke the same morphological response from sporangiospores, arthrospores, and vegetative cells on solid as well as liquid growth media, researchers studying Mucor species with respect to their morphological responses to the environment have in some cases found enough variation to preclude any unqualified extrapolation of experimental results from one system to another.

Intracellular cAMP concentrations may play a role in the morphology of dimorphic fungi, and yeast morphology is characterised by an intracellular level of cAMP that has been reported about 3-fold higher than the level of cAMP in filamentous cells. An immediate target of cAMP is the cAMP-dependent protein kinase A (PKA). cAMP is also known to act primarily as an effector of PKA in eukaryotic cells.

Extensive knowledge has been gained about PKA in a variety of experimental systems (for review, see Taylor et al., 1990). PKA consists of two regulatory subunits (PKAR) that bind to and inhibit the activity of two catalytic subunits (PKAC). In the presence of cAMP, PKAR dissociates from PKAC, resulting in free catalytic subunits that are the active kinase.

The catalytic core of PKAC contains two well conserved regions: an ATP-binding site and a serine/threonine protein kinase active site. The ATP-binding site is a glycine-rich stretch of residues (GXGXXG) in the vicinity of a lysine residue. The active site is located in the central part of the catalytic domain and contains a conserved apartic acid residue, which is important for the catalytic activity of the enzyme. In contrast, the N-termini of PKACs are quite variable. In PKAR, three major structural features are present. First, a dimerisation domain located at the N-terminal one-third of the protein that mediates dimer formation between two PKAR subunits and interaction with other proteins. Second, a highly conserved six-residue sequence (consensus RRtSVs) kinase inhibitor domain that mediates interaction between PKAR and PKAC and acts as an inhibitor of PKAC kinase activity. The third feature of PKAR is the presence of two near-duplicate cAMP binding domains, which are also highly conserved.

The regulation of PKA activity is inversely proportional to the concentration of cAMP. Binding of cAMP to PKAR results in the release of PKAC subunits and the triggering of a kinase cascade resulting in morphogenesis, differentiation and dimorphism. Earlier work identified two species of cAMP-binding proteins in M. circinelloides and M. genevensis with similar molecular weight (51- or 65-kD; Forte and Orlowski 1980). At least one of these protein species may represent PKAR. Remarkably, in the related fungus M. rouxii, PKAR was identified as a 70-kDa protein (Moreno and Passeron 1980).

The involvement of PKA in polarized growth has been shown using of cAMP analogues. Addition of these analogues mimics activation of PKAC and results in Mucor yeast growth under aerobic conditions. Likewise, the regulation of PKA activity is crucial throughout the filamentation phase, since the removal of analogues results in the immediate shift from isodiametric to filamentous growth (Rossi et al., 1994; Orlowski 1991). Recently, cloning of the cAMP binding domains of the M. rouxii PKAR and recombinant production in E. coli has been reported (Sorol et al., 2000).

Addition of cAMP analogues has also been shown to repress the de novo synthesis of MRAS3, one of the three RAS proteins found in Mucor (Roze et al 1999). The RAS superfamily of small GTP-binding proteins includes signal-coupling proteins which are components of an intracellular signaling network mediating an appropriate cellular response to external stimuli. MRAS3 is mainly associated with polar growth, as well as being involved in other processes like sporulation and germination, while MRAS1 is only associated with the regulation of polar growth (Roze et al 1999). As for PKA activity, activation of a RAS-MAP kinase pathway in S. cerevisiae and C. albicans leads to high cAMP levels and pseudohyphal development, whereas in Mucor and in the maize pathogen Ustilago maydis high levels of cAMP constrain growth to the yeast form (Orlowski 1991; Borges-Walmsley and Walmsley 2000).

A few examples of heterologous protein production have been described for Mucor circinelloides. The production in M. circinelloides of a Mucor miehei aspartic protease (MmAP) represents one example of recombinant protein production in Mucor (Dickinson et al., 1987). In this case, the native promoter and the secretion signal of MmAP were used. Attempts to produce recombinant calf chymosin in M. circinelloides are also reported for the same expression and secretion signals, although the levels obtained were extremely low (Stroman et al., 1990). In both cases, filamentous growth on solid medium was preferred for protein production and secretion.

Other examples of recombinant proteins produced in M. circinelloides are limited to enzymes involved in biosynthesis of amino acids and carotenes. Direct selection in an auxotrophic host (Iturriaga et al., 1992) or expression of homologous genes involved in carotenogenesis has been exploited (Ruiz-Hidalgo et al., 1999; Navarro et al., 2000). In these cases, the natively associated signals were used to express the gene of interest.

A dimorphic fungal cell, Arxula adeninivorans, has recently been developed for the production of recombinant proteins (Wartmann et al., 2000). The type of morphology is dependent of the growth temperature. Filamentous growth is obtained by increasing temperature above 42° C. This approach has been proposed solely to enhance secretion of the recombinant protein. It is assumed in the art that filamentous fungi have a larger secretion capacity than yeasts, although interspecific comparisons are somewhat cumbersome.

However, the proposed use of a temperature shift is normally associated with the triggering of a heat shock response in the cell. Among the physiological and cellular effects of heat shock, the synthesis of a number of stress proteases is initiated and maintained during the period of growth at high temperature and the degradation of heat denatured proteins is very effectively carried out.

WO 93/08285 relates to psedohyphal growth of yeasts including Saccharomyces species incapable of true filamentous growth. Whereas vegetative growth by filamentous fungi is by hyphal elongation, vegetative growth by yeasts such as Saccharomyces is by budding of a unicellular thallus. The filamentous fungi of the present invention are thus morphologically, physiologically, and genetically distinct from yeasts.

### SUMMARY OF THE INVENTION

Although dimorphic fungi including Mucor species are capable of growing either as unicellular, essentially spherical cells, or as a filamentous fungi, the shift between growth as essentially spherical cells and filamentous growth requires the activation of a complex genetic machinery that is at present not well understood. This lack of understanding has prevented the morphological characteristics of dimorphic fungi from being exploited in biotechnological processes, e.g. when dimorphic fungi are used as host organisms for heterologous gene expression.

One way of controlling the morphology of Mucor sp. is to control the growth conditions. Thus, high glucose concentrations and anaerobic conditions favour growth of Mucor sp. as unicellular, essentially spherical cells. However, under these growth conditions high levels of ethanol is produced and results in growth inhibition and low biomass yields. In order to overcome this problem, the controlled expression of one or more key genes involved in the control of dimorphism or filamentation is preferred.

Accordingly, there exists a need for industrially applicable, dimorphic fungi capable of shifting morphology—rapidly and controlably—from a single cell morphology to a morphology characterised by a filamentous growth. In particular, there exists a need for such cells having an improved filamentation capability resulting in an increased production and/or secretion of a desirable polypeptide.

The lack of suitable genetic tools such as strong and regulated promoters has represented a significant drawback for the utilization of Mucor for production of heterologous proteins. However, the fact that Mucor is capable of growing either as a yeast or as a filamentous fungal cell represents a technological advantage, provided the shift between the two growth types can be made based on a set of readily controllable parameters.

It is an object of the present invention to provide fungal host organisms capable of expressing recombinant proteins while at the same time exhibiting satisfactory growth characteristics.

It is a further object to provide—in a single fungal host organism—the characteristic of homogeneous growth and low viscosity typically associated with yeast organisms, and the capability for high protein secretion normally associated with filamentous fungi.

Accordingly, the present invention provides novel recombinant fungal host cells and a novel fermentation procedure in which the host strain is engineered in order to exhibit growth characteristics particularly well suited for growth in fermentors during the biomass production phase and for protein secretion during the heterologous protein production phase.

The host cells of the invention are capable of rapid growth as multipolar yeasts, which exhibit low viscosity even at a high biomass concentration and result in evenly dispersed cells allowing sufficient diffusion of nutrients. In particular, the host cells of the invention are dimorphic fungi, which under appropriate fermentation conditions grow as multipolar yeasts. When suitable levels of biomass are obtained, the filamentous growth is regulated by regulating the expression of at least one of a group of regulatory genes involved in the control of dimorphism and filamentation, wherein said at least one gene is operably linked to a suitable regulated promoter.

Preferred fungal hosts produce a homogeneous yeast culture under non-induced conditions. Most preferably, the fungal host cells are selected from the group consisting of Mucor sp., and other dimorphic Zygomycetes. Also other dimorphic fungi where control of the dimorphic shift can be regulated during growth in fermentor are preferred.

The invention also provides recombinant fungal host cells, as defined above, comprising a nucleic acid fragment encoding a heterologous protein (which is herein understood also to encompass peptides), which protein can be expressed by the host cell.

The invention further provides a method for production of heterologous proteins comprising the step of culturing a host cell of the invention under conditions conducive to the expression of the heterologous protein of interest, and comprising the further step of increasing the filamentation and/or controlling the induction of filamentation by increasing or decreasing the expression of at least one regulator of filamentation and dimorphism in any appropriate genetic background, and recovering the heterologous protein from the culture, including any recovery of the protein from the supernatant of the culture.

It is a further object of the invention to provide genetic tools useful for cloning and screening procedures in dimorphic fungi. Examples include strong and/or regulatable expression signals and selective markers. In particular, a novel vector allowing selection for high copy number is provided.

In one preferred aspect of the present invention, there is provided an isolated polynucleotide comprising

i) a first nucleotide sequence encoding at least one regulator of morphology capable of regulating the morphology of a dimorphic fungal cell, and operably linked thereto

ii) a second nucleotide sequence comprising an expression signal capable of directing the expression of the first nucleotide sequence in a dimorphic fungal cell,

wherein the first and second nucleotide sequences are not natively associated.

In another aspect, the present invention relates to a fungal host cell transformed or transfected with such a polynucleotide, wherein said fungal host cell optionally further comprises

i) at least one nucleotide sequence encoding a gene product, and operably linked thereto,

ii) at least one further nucleotide sequence comprising a further expression signal capable of directing the expression in a dimorphic fungal cell of the at least one nucleotide sequence encoding the gene product, wherein said further expression signal is regulatable, during growth of the dimorphic fungal cell, by one or more of

a) the composition of the growth medium, including at least one of carbon source, nitrogen source including amino acids or precursors thereof, oxygen content, ionic strength, including NaCl content, pH, low molecular weight compounds, cAMP, and the presence or absence of a cell constituent, or a precursor thereof,

b) the temperature of the growth medium, including any change thereof, including an upshift eliciting the expression of one or more heat shock genes,

c) the growth phase of the dimorphic fungal cell, and

d) the growth rate of the dimorphic fungal cell.

wherein the nucleotide sequence encoding the gene product and the further nucleotide sequence comprising the regulatable expression signal are not natively associated.

In a further aspect there is provided a dimorphic fungal cell comprising

i) at least one nucleotide sequence encoding a gene product, and operably linked thereto,

ii) at least one further nucleotide sequence comprising a further expression signal capable of directing the expression in a dimorphic fungal cell of the at least one nucleotide sequence encoding the gene product, wherein said further expression signal is regulatable, during growth of the dimorphic fungal cell, by one or more of

a) the composition of the growth medium, including at least one of carbon source, nitrogen source including amino acids or precursors thereof, oxygen content, ionic strength, including NaCl content, pH, low molecular weight compounds, cAMP, and the presence or absence of a cell constituent, or a precursor thereof,

b) the temperature of the growth medium, including any change thereof, including an upshift eliciting the expression of one or more heat shock genes,

c) the growth phase of the dimorphic fungal cell, and

d) the growth rate of the dimorphic fungal cell.

wherein the nucleotide sequence encoding the gene product and the further nucleotide sequence comprising the regulatable expression signal are not natively associated.

In a still further aspect, the above-mentioned dimorphic fungal cell is transfected or transformed with the polynucleotide comprising

i) a first nucleotide sequence encoding at least one regulator of morphology capable of regulating the morphology of a dimorphic fungal cell, and operably linked thereto

ii) a second nucleotide sequence comprising an expression signal capable of directing the expression of the first nucleotide sequence in a dimorphic fungal cell,

wherein the first and second nucleotide sequences are not natively associated.

In further aspects, the present invention relates to a method for constructing a recombinant fungal cell, or a recombinant dimorphic fungal cell, said method comprising the step of transforming or transfecting a polynucleotide, or a vector comprising said polynucleptide, into a fungal cell or a dimorphic fungal cell.

The method for constructing a recombinant fungal cell, or a recombinant dimorphic fungal cell, preferably comprises the further steps of

transforming or transfecting said recombinant fungal cell or said recombinant dimorphic fungal cell with a further polynucleotide comprising

i) at least one nucleotide sequence encoding a gene product, and operably linked thereto, and

ii) at least one further nucleotide sequence comprising a further expression signal capable of directing the expression in a dimorphic fungal cell of the at least one nucleotide sequence encoding the gene product, wherein said further expression signal is regulatable, during growth of the dimorphic fungal cell, by one or more of

a) the composition of the growth medium, including at least one of carbon source, nitrogen source including amino acids or precursors thereof, oxygen content, ionic strength, including NaCl content, pH, low molecular weight compounds, cAMP, and the presence or absence of a cell constituent, or a precursor thereof,

b) the temperature of the growth medium, including any change thereof, including an upshift eliciting the expression of one or more heat shock genes,

c) the growth phase of the dimorphic fungal cell, and

d) the growth rate of the dimorphic fungal cell,

wherein the nucleotide sequence encoding the gene product and the further nucleotide sequence comprising the regulatable expression signal are not natively associated.

In a further aspect the present invention provides a method for regulating the morphology of a recombinant fungal cell or a recombinant dimorphic fungal cell, said method comprising the steps of

i) cultivating said fungal cell or said dimorphic fungal cell under conditions allowing expression of said first nucleotide sequence encoding the at least one regulator of morphology, and

ii) regulating the morphology of said recombinant fungal cell or said recombinant dimorphic fungal cell, wherein said regulation of the morphology results from regulating the expression in said recombinant fungal cell, or said recombinant dimorphic fungal cell, of said at least one regulator of morphology.

In a further aspect there is provided a method for obtaining a predetermined dimorphic shift of a dimorphic fungal cell, said method comprising the steps of

i) cultivating said dimorphic fungal cell under conditions allowing expression of said first nucleotide sequence encoding the at least one regulator of morphology, and

ii) obtaining a predetermined dimorphic shift of said dimorphic fungal cell, wherein said dimorphic shift results from regulating the expression in said dimorphic cell of said at least one regulator of morphology.

In a still further aspect the present invention provides a method for increasing the filamentation of a fungal cell, or a dimorphic fungal cell, said method comprising the steps of

i) cultivating said fungal cell, or said dimorphic fungal cell, under conditions allowing expression of said first nucleotide sequence encoding the at least one regulator of morphology, and

ii) increasing the filamentation of said fungal cell, or said dimorphic fungal cell, wherein said increased filamentation results from regulating the expression in said dimorphic cell of said at least one regulator of morphology.

In a still further aspect there is provided a method for increasing the secretory capacity of a fungal cell, or a dimorphic fungal cell, said method comprising the steps of

i) cultivating said fungal cell, or said dimorphic fungal cell, under conditions allowing expression of said first nucleotide sequence encoding the at least one regulator of morphology, and

ii) increasing the secretory capacity of said fungal cell, or said dimorphic fungal cell, wherein said increased secretory capacity results from regulating the expression in said dimorphic cell of said at least one regulator of morphology.

In an even further aspect there is provided a method for producing a gene product in a fungal cell, or a dimorphic fungal cell, said method comprising the steps of

i) cultivating said fungal cell, or said dimorphic fungal cell, under conditions allowing expression of said first nucleotide sequence encoding said at least one regulator of morphology, and

ii) cultivating said fungal cell, or said dimorphic fungal cell, under conditions allowing expression of said nucleotide sequence encoding said gene product, and

iii) producing the gene product.

### BRIEF DESCRIPTION OF THE DRAWINGS

FIG. 1

Following a shift from anaerobic to aerobic growth conditions, a transition of Mucor circinelloides morphology gradually occurs from a unicellular, essentially spherical morphology to filamentous structures characterised by an aseptate mycelium comprising multinucleated cells. Panels 1 to 4 illustrate the unicellular, essentially spherical morphology of M. circinelloides that can be observed during anaerobic growth (panel 1) and immediately after the shift to aerobic growth (panels 2-4, representing the first 3 h after the shift). Panels 5-6 illustrate the subsequent development of numerous protruding structures which evolve into hyphae. Hyphal development (i.e., elongation) occurs rapidly (panels 7-8). Panels 9-11 illustrate that branching of hyphae becomes evident and proliferates following the first 10 h after the shift.

FIG. 2

Signal transduction pathways controlling filamentation and dimorphism in fungi. Two major pathways are depicted, the cAMP dependent (cAMP boxes) and the MAP kinase pathway (MAPK boxes). The gene nomenclature for each organism is used and the common denomination is shown below in brackets for comparison.

FIG. 3

Features of the M. circinelloides pkaR promoter: Putative CAAT boxes are shown in uppercase; a putative TATA box and a CT-rich stretch are depicted underlined; the start of the coding region is shown in uppercase with the translated protein in one-letter code.

FIG. 4

Multiple alignment of PKARs. The M. circinelloides PKAR was aligned with other fungal PKAR sequences. Identical residues are boxed. Abbreviations and accession numbers: Mcir: M. circinelloides, AJ400723 (EMBL); Anig: Aspergillus niger, Q9C196 (SwissProt); Beme: Blastocadiella emersonii, P31320 (SwissProt); Calb: C. albicans Q9HEW1 (SwissProt); Scer: S. cerevisiae, P07278 (SwissProt), Spom: Schizosaccharomyces pombe, P36600 (SwissProt); Mrou: M. rouxii, Q9P8K6 (SwissProt).

FIG. 5

Alignment of M. circinelloides PKAC with other fungal counterparts. Identical residues are boxed. Abbreviations and accession numbers: M.cir: M. circinelloides, AJ400723 (EMBL); Anig: A. niger, P87077 (SwissProt); Beme: B. emersonii, Q12741 (SwissProt); Calb: C. albicans Q9HEW0 (SwissProt); Scer: S. cerevisiae, P06245 (SwissProt), Spom: S. pombe, P40376 (SwissProt).

FIG. 6

Expression of pkaR and pkaC. A: Expression of pkaR and pkaC under anaerobic and aerobic growth conditions analysed by Northern blotting. RNA was isolated from overnight cultures of strain R7B grown anaerobically (lanes 1 and 3) or aerobically (lanes 2 and 4) in SIV medium. B: Expression of pkaR during shift from anaerobic to aerobic growth conditions. Northern blot analysis using RNA obtained from R7B growing anaerobically in Vogel's medium (lane 1) and the same culture 4 h after the shift to aerobic conditions without (lane 2) or with the simultaneous addition of 2, 5, or 10% glucose (lanes 3, 4, and 5, respectively). RNA gels were shown below for loading control.

FIG. 7

Overexpression of PKAR in M. circinelloides. A: Plasmid map (left) of pEUKA4-pkaR. Northern blot analysis (middle panel) of KFA121 (a pEUKA4-pkaR transformant) grown in YNB medium with 5% glucose (lane 2). The same conditions were used for the control strain KFA89 (lane 1). The RNA gel is shown below for loading control. Primer extension analysis (right panel): the fragment obtained is indicated with an arrow; a sequence ladder was run on pEUKA4-pkaR to determine the transcription start site (tss). The sequence obtained is shown below (the arrow indicates the tss; mRNA sequence is shown in italics, cloning site (XhoI) and ATG start codon of pkaR (bold). B: Colony morphology of KFA121 (right) and KFA89 (left) on YNB plates (2% glucose) showing the higher branching degree of KFA121.

FIG. 8

Growth morphology of M. circinelloides during anaerobic growth at different glucose concentrations. Anaerobic growth of strain R7B on YNB medium with 0.1% or 0.5% glucose (left and right panels, respectively).

FIG. 9

The M. circinelloides STE12 and MPK1 homologues. A: The protein sequence corresponding to the identified M. circinelloides ste12 fragment was aligned with relevant fungal STE12 sequences. Abbreviations and accession numbers: Mcir: M. circinelloides, AJ400723 (EMBL); A.nid: A. nidulans, O74252 (SwissProt); Calb: C. albicans P43079 (SwissProt); Scer: S. cerevisiae, P13574 (SwissProt). B: The protein sequence corresponding to the identified M. circinelloides mpk1 fragment was aligned with relevant fungal STE12 sequences. Abbreviations and accession numbers: Mcir: M. circinelloides, AJ400723 (EMBL); Calb: C. albicans P43068 (SwissProt); Scer: S. cerevisiae, Q00772 (SwissProt), Spom: S. pombe, Q92398 (SwissProt).

FIG. 10

Nucleotide sequence and derived amino-acid sequence of gpd1. Numbering of nucleotides is with respect to the start of the coding sequence. Exon sequences are capitalised. Sequences with homology to the lariat formation consensus sequence within introns are italicised. Putative TATA and CAAT boxes are boxed and bolded, respectively. Pyrimindine stretch is underlined. The putative polyadenylation signal is double underlined. The transcription start point is capitalised and bolded. The sequence corresponding to the gene-specific oligonucleotide used in Northern blotting and primer extension is wavy underlined.

FIG. 11

Northern blot analysis of the expression of gpd1, gpd2 and gpd3. A: Lane 1-3: 1.1 kb DNA fragments of gpd1, gpd2 and gpd3, respectively. Y: RNA isolated from M. circinelloides growing anaerobically as yeasts in the presence of glucose. M: RNA isolated from M. circinelloides shifted from anaerobic to aerobic conditions at the time of glucose depletion and further incubated for four hours allowing the initiation of mycelial growth. Filters 1, 2 and 3 were hybridized with oligonucleotides specific for gpd1, gpd2 and gpd3, respectively. B: Micrographs taken from the yeast (Y) and mycelial (M) culture.

FIG. 12

Northern blot analysis of the expression of gpd1. A: Total RNA was isolated from M. circinelloides grown aerobically in shake flask in rich medium with either glucose (Glu), glycerol (Gly) or ethanol (EtOH) added. B: Total RNA was isolated from M. circinelloides grown anaerobically in fermentor in rich medium with either glucose or galactose. Samples were taken with 2-4 hours interval until sugar was depleted. Sugar concentrations are indicated in the boxes. After depletion of glucose, additional glucose was added and a sample was taken 1 hour after the readdition. The blots were hybridized with a 1.1 kb gpd1 probe.

FIG. 13

Recombinant expression. A: Expression plasmids pEUKA4-crgA and pEUKA4-gox1 (EMBL accession nos. AJ305344 and AJ305345, respectively). B: M. circinelloides R7B transformed with pEUKA4-crgA, and as a control R7B carrying pEUKA4-gox1, grown in the dark for 3 days. Arrows indicate yellow colonies. C: Zymogram of culture supernatants. M. circinelloides harboring the expression plasmid pEUKA4-gox1 was inoculated in SIV medium containing 2% or 5% glucose and incubated under aerobic conditions. Samples were taken after 40 hours of incubation where glucose had not yet been depleted. GOX activity in culture supernatants was analyzed by native gel electrophoresis and staining for enzyme activity.

FIG. 14

Zymogram of culture supernatants. M. circinelloides R7B harboring the expression plasmid pEUKA4-gox1 was inoculated in 6×SIV medium containing 5% glucose and grown in fermentors: A: under anaerobic conditions, B: initially anaerobic conditions, then shifted to aerobic conditions (the dotted line separates samples taken before and after the shift), C: under aerobic conditions. Samples were taken between 16 and 45 hours after inoculation. GOX activity in culture supernatants was analyzed by native gel electrophoresis and staining for enzyme activity. Biomass (g dry weight pr. kg culture) and glucose concentration (g/l) of the culture was determined for each sample. D: Western blot analysis of culture supernatants. Samples from the anaerobic (1-4), shifted (5-8) and aerobic 9-12) cultures were subjected to Western blot analysis using GOX-specific antibodies. Commercial GOX (C) was used as positive control.

FIG. 15

Expression plasmid pEUKA8-gox1.

FIG. 16

Zymogram of culture supernatants. M. circinelloides R7B harboring the expression plasmid pEUKA8-gox1 was inoculated in 6×SIV medium containing 5% glucose and grown in fermentors: A: under anaerobic conditions, B: initially anaerobic conditions, then shifted to aerobic conditions (the dotted line separates samples taken before and after the shift), C: under aerobic conditions. Samples were taken between 18 and 60 hours after inoculation. GOX activity in culture supernatants was analyzed by native gel electrophoresis and staining for enzyme activity. Commercial GOX (C) was used as positive control. Biomass (g dry weight pr. kg culture) was determined for each sample.

FIG. 17

Spores of Mucor circinelloides strain R7B were plated on to YNB agar pH 6.0 supplemented with leucine (upper left plate in all panels) or the same medium supplemented with 50, 100 or 250 μg/ml of the antibiotic (geneticin, kanamycin, hygromycin B or neomycin) as indicated in each panel. After 4 days at 28° C., plates were examined and photographed.

FIG. 18

A vector for high copy number plasmids in M. circinelloides. A: plasmid pEUKA7-kan: this plasmid is a derivative of pEUKA4-gox1 (Wolff and Arnau, 2002) where the gox1 gene and the trpC terminator are replaced with the kan gene from vector pCR2.1 and the terminator of the M. circinelloides gpd1 gene, respectively; B: Southern blot analysis of PstI-digested DNA from strain KFA143 (a pEUKA7-kan transformant) grown in YNB without leucine (lane 1), or YNB with leucine and 100, 250 or 500 mg/ml geneticin (lanes 2, 3 and 4 respectively). A fragment of the M. circinelloides leuA gene was used as a probe. The chromosomal leuA locus is located in a 4.4 kb PstI fragment (depicted as ‘chromosome’) and the plasmid leuA is placed in a 5.1 kb fragment (‘plasmid’); C: Plasmid pEUKA11: Removal of approx. 1.7 kb unnecessary DNA from pEUKA7-kan and inclusion of a multi-cloning site allow the use of this vector to construct genomic libraries.

FIG. 19

Growth curves for S. cerevisiae strain MDO39 (vector control) and MDO41 (pkaR overexpressing strain) in SC medium supplemented with glucose (glu) or with galactose and raffinose (gal raf).

FIG. 20

Colony morphology of S. cerevisiae strain MDO39 (vector control) and MDO41 (pkaR overexpression strain) on SC medium. Top: typical yeast colony morphology on glucose-containing medium for MDO39 and MDO41. A series of pictures were taken 4 hours after the addition of a glucose drop to SC plates containing galactose and raffinose from the position of the drop (i.e., the first picture was taken at the position of the drop [e.g., MDO39 gal-1], the second half way through the plates [e.g., MDO39 gal-2] and the third at the opposite end of the plate [e.g., MDO39 gal-3]).

FIG. 21

Colony morphology of Rhizomucor pusillus transformed strains KFA183 (crgA overexpressing control) and KFA185 (pkaR). The strains were grown overnight in YNB medium supplemented with 1% or 5% glucose (left and right panels as indicated).

FIG. 22

Expression of the Mucor circinelloides tubA gene. Northern blot (top panel) using RNA isolated from strain R7B grown anaerobically (lane 1) or aerobically (lane 2) in YPG medium. The RNA gel stained with ethidium bromide is shown as a loading control (bottom panel).

FIG. 23

Expression of the Mucor circinelloides gal1 gene. Northern blot (top panel) using RNA isolated from strain R7B grown in YP medium containing different carbon sources and a gal1-specific probe. Lane 1: 2% glucose; lane 2: 2% galactose; lane 3: 2% glucose and 2% galactose; lane 4: 2% glycerol; lane 5: no additional carbon source added. The RNA gel stained with ethidium bromide is shown as a loading control (bottom panel).

### DETAILED DESCRIPTION OF THE INVENTION

The commercial use of any recombinant protein largely depends on the ability to achieve efficient production in large-scale fermentation, and productivity is limited by a number of factors in industrial fermentation of fungi.

The common problems associated with the use of filamentous fungi as hosts are related to the relatively high viscosity as compared to unicellular organisms, such as Saccharomyces cerevisiae and Bacillus sp., and the often very heterogeneous distribution of mycelium in dense aggregates causing a majority of the mycelium to starve, due to lack of O₂ and/or nutrient diffusion to all of the cells.

The high viscosity reduces the oxygen transfer rate that can be reached in the fermentor, which in turn adversely effects the overall energy the cells can produce, thereby leading to lower concentration of obtainable productive biomass and lower final product yield or longer fermentation times. It can therefore be seen that simply increasing biomass is not, without the proper morphology that leads to low viscosity, adequate to increase yield in fermentation. There must be an increase in productive biomass in order for any advantages to be obtained.

Dimorphic fungi do indeed exhibit certain growth characteristics, which render them suitable for culturing in fermentors. This group of fungi has the ability to reversibly switch between yeast and filamentous growth, typically as a response to a number of environmental stimuli. However, many of these organisms are relatively poorly characterized genetically and knowledge about the pathways controlling the dimorphic shift is very limited.

The present invention encompasses any dimorphic fungal cell, which can be used for the fermentation procedure as defined above. By “dimorphic fungal cell” is meant any fungal cell taxonomically belonging to the Eumycotina, including Zygomycetes, which is capable of displaying either a unicellular, essentially spherical morphology and/or a filamentous morphology characterised by a mycelium. This includes, but is not limited to, members of the genera Mucor. Further examples of taxonomic equivalents and other useful species can be found, for example, in Cannon, Mycopathologica 111: 75-83, 1990; Moustafa et al., Persoonia 14: 173-175, 1990; Stalpers, Stud. Mycol. 24, 1984; Upadhyay et al., Mycopathologia 87: 71-80, 1984; Subramanian et al., Cryptog. Mycol. 1: 175-185, 1980; Guarro et al., Mycotaxon 23: 419-427, 1985; Awao et al., Mycotaxon 16: 436-440, 1983; von Klopotek, Arch. Microbiol. 98:365-369, 1974; and Long et al., 1994, ATCC Names of Industrial Fungi, ATCC, Rockville, Md. Those skilled in the art will readily recognize the identity of appropriate equivalents.

As the results presented in the examples show, several strains may possess the morphology required to make them useful in the fermentation procedure described herein. Thus, it is understood that the utility is not limited to a single isolate or strain, but rather is a characteristic of a group of species. Those skilled in the art will recognize that other strains or isolates of these species can also be used in expression of heterologous expression. Many strains of Mucor circinelloides species are publicly available in the collections of the American Type Culture Collection (ATCC) 12301 Parklawn Drive, Rockville Md. 20852.

Suitability of other dimorphic fungal hosts for use in fermentors can be determined by the methods described in the following examples. Briefly, candidate fungi are cultured on standard growth medium such as salts/yeast extract, soy, potato protein, or any medium supplemented with glucose or other appropriate carbon source. The fermentation is carried out at a pH of about 4-7 and at a temperature of from about 25° C. to 35° C. It will be recognized that the temperature of the control fermentation should be that which is optimal for the control strain; for M. circinelloides, this is about 28° C.

Useful fungal strains should be able to switch between a unicellular, essentially spherical morphology and a filamentous morphology characterised by a mycelium in response to a variety of environmental and nutritional conditions. Confirmation of utility is best determined in fermentors, by evaluating actual viscosity of the culture at various time points in the fermentation. Viscosity determination can be made by any means known in the art, e.g., Brookfield rotational viscometry (defined or unlimited shear distance and any type of spindle configuration), kinematic viscosity tubes (flow-through tubes), failing ball viscometer or cup-type viscometer.

As noted above, Mucor circinelloides is, because of its excellent dimorphic morphology, among the preferred species for use in recombinant protein production. However, this species, in anaerobic cultures grown without nutrient limitation adopts a unicellular, essentially spherical morphology that results in low biomass production. This is due, among other things, to growth inhibition caused by accumulation of ethanol.

Aerobic growth of Mucor circinelloides results in mycelia, which are the preferred source of production of the recombinant proteins. The mycelia of filamentous fungi have a naturally high capacity for protein secretion due to their saprophytic lifestyle, and protein secretion in filamentous fungi occurs at the hyphal tips (Gordon et al 2000; Wösten et al 1991).

The present invention combines the advantages of both of the above-mentioned growth morphologies without compromising biomass production or protein secretion.

Fungal Cells the Morphology of which Is Regulatable by a Regulator of Morphology

When the present invention in one aspect relates to an isolated polynucleotide comprising

i) a first nucleotide sequence encoding at least one regulator of morphology capable of regulating the morphology of a dimorphic fungal cell, and operably linked thereto

ii) a second nucleotide sequence comprising an expression signal capable of directing the expression of the first nucleotide sequence in a dimorphic fungal cell,

wherein the first and second nucleotide sequences are not natively associated,

the dimorphic fungal cell, the morphology of which is regulatable by at least one regulator of morphology, is preferably capable of growing as i) a multinucleated cell having a unicellular, essentially spherical morphology and/or ii) a mycelium having a filamentous structure and comprising multinucleated cells. The different morphologies can be observed individually under appropriate growth conditions, and both forms of morphology can be observed in connection with a dimorphic shift. The unicellular dimorphic fungal cells are in one embodiment multinucleated and optionally multipolar, but they may also be bipolar or monopolar.

It will be understood that the dimorphic fungal cells mentioned herein immediately above and below are in one embodiment fungal cells, the morphology of which is regulatable by the at least one regulator of morphology. Such fungal cells thus characterise in functional terms the polynucleotide according to the invention comprising the first nucleotide sequence encoding the at least one regulator of morphology and the second nucleotide sequence comprising the expression signal, wherein said first and second nucleotide sequences are not natively associated.

In another embodiment, the dimorphic cells listed herein below are also comprised by the term “host cell”, wherein such a host cell is transfected or transformed with the polynucleotide according to the invention comprising said first and second not natively associated nucleotide sequences, wherein said second nucleotide sequence is capable of directing expression of the first nucleotide sequence in such a host cell. Furthermore, “host cells” shall also be understood to comprise fungal cells that are not dimorphic fungal cells.

When regulating the morphology of a dimorphic fungal cell, the increased or decreased production of the at least one regulator of morphology preferably results in a dimorphic shift of the dimorphic fungal cell and/or in a filamentous morphology of the dimorphic fungal cell and/or an improved filamentation of the dimorphic cell.

When regulating the morphology of a fungal cell that is not a dimorphic cell, the increased or decreased production of the at least one regulator of morphology in such a fungal cell preferably results in a filamentous morphology of the fungal cell and/or an improved filamentation of the fungal cell.

Improved or increased filamentation of a fungal cell including a dimorphic fungal cell is defined herein as any one of i) the transition of a single cell from a unicellular, essentially spherical morphology to a mycelium having a filamentous morphology; ii) an increase in the proportion of cells having a filamentous morphology; iii) an increased branching frequency (hyperbranching); and iv) a decreased hyphal growth unit (HGU) length.

The HGU is the total length (or area) of a hyphal element divided by the number of tips. The growth unit of a mycelium clearly differs qualitatively from the “growth units” (i.e. cells) of a unicellular fungal cell having an essentially spherical morphology. Branch initiation in the mycelium of a filamentous fungal cell is regulated during filamentous growth. When the mean HGU value of a mycelium (total hyphal length of the mycelium divided by its total number of tips) exceeds a critical value, a new branch is initiated. A unicellular fungal cell having an essentially spherical morphology is defined has having an HGU of 0, and such a cell will only obtain a HGU if it starts to produce filaments. Increased branching will result in a decrease of HGU.

There is a linear relation between the HGU length and culture viscosity (Bocking et al. 1999). According to one hypothesis, the number of hyphal tips per unit biomass is maximal at early stages of filamentous growth, whereas at later times their numbers do not increase in proportion to the biomass (Chaudhuri et al 1999).

Consequently, the at least one regulator of morphology can be expressed in both a dimorphic fungal cell and a fungal cell that is not capable of displaying dimorphic morphology. The result of regulating the production of the at least one regulator is preferably an improved filamentation, and such an improved filamentation in one embodiment occurs in a dimorphic fungal cell independently of a dimorphic shift. This will e.g. be the case when the at least one regulator is being produced in a dimorphic cell growing as a mycelium or adopting a filamentous morphology, wherein said increased or decreased production of regulator of morphology results in an improved filamentation. An increased expression or a decreased expression is an expression that is altered as compared to the expression of the at least one regulator directed by the native expression signal of said regulator.

In one embodiment, the dimorphic fungal cell, the morphology of which is regulatable by the at least one regulator of morphology, belongs to the class of Zygomycetes, including the order of Mucorales, including from the order of Mucorales a genus selected from the group of genera consisting of Mucor, Thermomucor, Rhizomucor, Mycotypha, Rhizopus, and Cokeromyces, including Cokeromyces recurvatus. Another preferred dimorphic fungal cell is Yarrowia lipolytica.

It is preferred in one embodiment that the dimorphic fungal cell, the morphology of which is regulatable by the at least one regulator of morphology, belongs to the genus Mucor. Preferred Mucor species includes, but is not limited to M. circinelloides, M. hiemalis, M. rouxii, M. genevensis, M. bacilliformis, and M. subtillissimus. M. circinelloides is particularly preferred.

Additionally preferred Mucor species, the morphology of which is regulatable by the regulator of morphology, includes, but is not limited to Mucor abundans, Mucor adventitius=Syn. of Mucor hiemalis f. hiemalis, Mucor adventitius var. aurantiacus=Syn. of Mucor hiemalis f. hiemalis, Mucor alboater=Syn. of Mucor piriformis, Mucor aligarensis, Mucor amphibiorum, Mucor angulisporus=Syn. of Mortierella ramanniana var. angulispora, Mucor ardhlaengiktus, Mucor aromaticus=Syn. of Mucor recurvus var. recurvus, Mucor assamensis=Syn. of Hyphomucor assamensis, Mucor attenuatus=Syn. of Mucor flavus, Mucor azygosporus, Mucor bacilliformis, Mucor bainieri, Mucor bedrchanii=Syn. of Mucor fuscus, Mucor botryoides=Syn. of Actinomucor elegans, Mucor botryoides var. minor=Syn. of Actinomucor elegans, Mucor brunneogriseus=Syn. of Mucor plumbeus, Mucor brunneus=Syn. of Mucor plumbeus, Mucor buntingii=Syn. of Rhizomucor pusillus, Mucor chibinensis=Syn. of Mucor racemosus f. chibinensis, Mucor christianiensis=Syn. of Mucor racemosus f. racemosus, Mucor circinans=Syn. of Pirella circinans, Mucor circinelloides f. circinelloides, Mucor circinelloides f. griseocyanus, Mucor circinelloides f. janssenii, Mucor circinelloides f. lusitanicus, Mucor coprophilus=Syn. of Mucor mucedo, Mucor corticolus=Syn. of Mucor hiemalis f. corticolus, Mucor corymbifer=Syn. of Absidia corymbifera, Mucor cunninghamelloides=Syn. of Actinomucor elegans, Mucor cylindrosporus=Syn. of Mucor microsporus, Mucor dimorphosporus=Syn. of Mucor racemosus f. racemosus, Mucor dispersus=Syn. of Backusella lamprospora, Mucor dispersus var. megalosporus=Syn. of Mucor zychae var. linnemanniae, Mucor dubius=Syn. of Mucor circinelloides f. circinelloides, Mucor falcatus, Mucor flavus, Mucor fragilis, Mucor fuscus, Mucor genevensis, Mucor gigasporus, Mucor globosus=Syn. of Mucor racemosus f. sphaerosporus, Mucor glomerula=Syn. of Actinomucor elegans, Mucor grandis, Mucor griseobrunneus=Syn. of Mucor fuscus, Mucor griseocyanus=Syn. of Mucor circinelloides f. griseocyanus, Mucor griseocyanus f. janssenii=Syn. of Mucor circinelloides f. janssenii, Mucor griseolilacinus=Syn. of Mucor circinelloides f. lusitanicus, Mucor griseoochraceus=Syn. of Mucor mucedo, Mucor griseoochraceus var. minuta=Syn. of Mucor minutus, Mucor griseoroseus=Syn. of Mucor circinelloides f. circinelloides, Mucor guilliermondii, Mucor hiemalis, Mucor hiemalis f. corticolus, Mucor hiemalis f. hiemalis, Mucor hiemalis f. luteus, Mucor hiemalis f. silvaticus, Mucor hiemalis var. albus=Syn. of Mucor hiemalis f. hiemalis, Mucor hiemalis var. flavus=Syn. of Mucor hiemalis f. hiemalis, Mucor hiemalis var. griseus=Syn. of Mucor hiemalis f. hiemalis, Mucor hiemalis var. toundrae=Syn. of Mucor hiemalis f. hiemalis, Mucor humicolus=Syn. of Mucor hiemalis f. hiemalis, Mucor inaequisporus, Mucor indicae-seudaticae=Syn. of Thermomucor indicae-seudaticae, Mucor indicus, Mucor janssenii=Syn. of Mucor circinelloides f. janssenii, Mucor jauchae=Syn. of Mucor circinelloides f. lusitanicus, Mucor javanicus=Syn. of Mucor circinelloides f. circinelloides, Mucor kanivcevii=Syn. of Mucor strictus, Mucor kurssanovii=Syn. of Mucor circinelloides f. janssenii, Mucor lamprosporus=Syn. of Backusella lamprospora, Mucor lausannensis=Syn. of Mucor hiemalis f. hiemalis, Mucor laxorrhizus, Mucor laxorrhizus var. ovalisporus, Mucor lusitanicus=Syn. of Mucor circinelloides f. lusitanicus, Mucor luteus=Syn. of Mucor hiemalis f. luteus, Mucor luteus var. indica=Syn. of Mucor variisporus, Mucor mandshuricus=Syn. of Mucor circinelloides f. circinelloides, Mucor mephitis=Syn. of Mucor flavus, Mucor meridionalis=Syn. of Mucor flavus, Mucor microsporus, Mucor miehei=Syn. of Rhizomucor miehei, Mucor minutus, Mucor mirus=Syn. of Mortierella isabellina, Mucor mousanensis, Mucor mucedo, Mucor mucilagineus=Syn. of Mucor plasmaticus, Mucor murorum=Syn. of Mucor mucedo, Mucor nanus, Mucor norvegicus=Syn. of Rhizopus oryzae, Mucor oblongiellipticus, Mucor oblongisporus, Mucor odoratus, Mucor ovalisporus=Syn. of Mucor aligarensis, Mucor peacockensis=Syn. of Mucor flavus, Mucor petrinsularis=Syn. of Mucor fuscus, Mucor petrinsularis var. echinosporus=Syn. of Mucor fuscus, Mucor petrinsularis var. ovalisporus=Syn. of Mucor aligarensis, Mucor pirelloides=Syn. of Pirella circinans, Mucor piriformis, Mucor pispekii=Syn. of Mucor racemosus f. racemosus, Mucor plasmaticus, Mucor plumbeus, Mucor plumbeus var. globosus=Syn. of Mucor racemosus f. sphaerosporus, Mucor plumbeus var. intermedius=Syn. of Mucor fuscus, Mucor plumbeus var. levisporus=Syn. of Mucor racemosus f. sphaerosporus, Mucor prainii=Syn. of Mucor circinelloides f. circinelloides, Mucor prayagensis, Mucor pseudolamprosporus=Syn. of Backusella circina, Mucor psychrophilus, Mucor pusillus=Syn. of Rhizomucor pusillus, Mucor pyri=Syn. of Mucor racemosus f. sphaerosporus, Mucor racemosus f. brunneus=Syn. of Mucor racemosus f. racemosus, Mucor racemosus f. chibinensis, Mucor racemosus f. racemosus, Mucor racemosus f. sphaerosporus, Mucor ramannianus=Syn. of Mortierella ramanniana var. ramanniana, Mucor ramificus=Syn. of Mucor circinelloides f. circinelloides Mucor ramiger=Syn. of Mucor fuscus, Mucor ramosissimus, Mucor recurvus, Mucor recurvus var. indicus, Mucor recurvus var. recurvus, Mucor rouxianus=Syn. of Mucor indicus, Mucor rouxii, Mucor rufescens=Syn. of Mucor odoratus, Mucor saturninus, Mucor saximontensis=Syn. of Zygorhynchus moelleri, Mucor sciurinus=Syn. of Mucor flavus, Mucor silvaticus=Syn. of Mucor hiemalis f. silvaticus Mucor sinensis, Mucor sphaerosporus=Syn. of Mucor racemosus f. sphaerosporus, Mucor spinescens=Syn. of Mucor plumbeus, Mucor spinosus=Syn. of Mucor plumbeus, Mucor strictus, Mucor subtilissimus, Mucor tauricus=Syn. of Rhizomucor tauricus, Mucor tenellus=Syn. of Mucor circinelloides f. janssenii Mucor thermophilus, Mucor tuberculisporus, Mucor ucrainicus, Mucor vallesiacus=Syn. of Mucor hiemalis f. hiemalis, Mucor variabilis, Mucor varians=Syn. of Mucor racemosus f. racemosus, Mucor variisporus, Mucor vesiculosus=Syn. of Gongronella butleri, Mucor wosnessenskii=Syn. of Mucor piriformis, Mucor zeicola=Syn. of Mucor circinelloides f. lusitanicus, Mucor zonatus, Mucor zychae var. linnemanniae, and Mucor zychae var. zychae (“=” denotes “same as”).

Although dimorphic fungal cells represent one group of fungal cells, the morphology of which is regulatable by the at least one regulator of morphology, the present invention is not limited to dimorphic fungal cells as host cells for the at least one regulator of morphology. The invention also relates to host cells in the form of any other fungal cell including any filamentous form of the subdivision Eumycotina. The fungal cells are characterized by a vegetative mycelium composed of chitin, cellulose, and other complex polysaccharides. Vegetative growth by filamentous fungi is by hyphal elongation. In contrast, vegetative growth by yeasts such as S. cerevisiae is by budding of a unicellular thallus. The filamentous fungi of the present invention are thus morphologically, physiologically, and genetically distinct from yeasts. Also, recent illustrations of differences between S. cerevisiae and filamentous fungi include the inability of S. cerevisiae to process Aspergillus and Trichoderma introns and the inability to recognize many transcriptional regulators of filamentous fungi.

Various species of filamentous fungi may be used as host cells in accordance with the present invention, including the following genera: Aspergillus, including Aspergillus niger and Aspergillus oryzae, Trichoderma, Neurospora, Podospora, Endothia, Mucor, Cochiobolus and Pyricularia. Specific expression hosts include A. nidulans (Yelton, M., et al., 1984, Proc. Natl. Acad. Sci. USA, 81:1470-1474; Mullaney, E. J. et al., 1985, Mol. Gen. Genet., 199:37-45; John, M. A. and J. F. Peberdy, 1984, Enzyme Microb. Technol., 6:386-389; Tilburn, et al., 1982, Gene, 26:205-221; Ballance, D. J., et al., 1983, Biochem. Biophys. Res. Comm., 112:284-289; and Johnston, I. L., et al., 1985, EMBO J., 4:1307-1311), A. niger (Kelly, J. M. and M. Hynes, 1985, EMBO, 4:475-479), A. awomari, e.g., NRRL 3112, ATCC 22342 (NRRL 3112), ATCC 44733, ATCC 14331 and strain UVK 143f, A. oryzae, e.g., ATCC 11490, N. crassa (Case, M. E., et al., 1979, Proc. Natl. Acad. Sci. USA, 76:5259-5263; and Lambowitz U.S. Pat. No. 4,486,533; Kinsey, J. A. and J. A. Rambosek, 1984, Molecular and Cellular Biology 4:117-122; Bull, J. H. and J. C. Wooton, 1984, Nature, 310:701-704); Trichoderma reesei, e.g. NRRL 15709, ATCC 13631, 56764, 56765, 56466, 56767, and Trichoderma viride, e.g., ATCC 32098 and 32086.

Further preferred fungal host cells are cells of the genus Cunninghamella, including Cunninghamella elegans and Cunninghamella polymorpha, cells of the genus Rhizomucor, including Rhizomucor miehei, Rhizomucor pusillus, Rhizomucor variabilitis, Rhizomucor variabilitis, and Rhizomucor variabilitis, cells of the genus Rhizopus, including Rhizopus oryzae, Rhizopus microsporus var. oligosporus, and Rhizopus niveus, cells of the genus Mortierella, including Mortierella isabelina, Mortierella verticillata, Mortierella alpina, and Mortierella vinacea.

In another embodiment, there is provided a polynucleotide comprising

i) a first nucleotide sequence encoding at least one regulator of morphology capable of regulating the morphology of a dimorphic fungal cell, and operably linked thereto

ii) a second nucleotide sequence comprising an expression signal capable of directing the expression of the first nucleotide sequence in a dimorphic fungal cell,

wherein the first and second nucleotide sequences are not natively associated, and wherein the dimorphic fungal cell, the morphology of which is regulatable by the at least one regulator of morphology, is capable of growing as a uninucleated cell having a unicellular, essentially spherical morphology, and/or capable of growing as a filamentous structure comprising uninucleated cells. Examples of such dimorphic fungi includes, but is not limited to Yarrowia, Candida and Arxula.

Origin of the First and/or Second Nucleotide Sequence

In yet another preferred embodiment, there is provided a polynucleotide comprising

i) a first nucleotide sequence encoding at least one regulator of morphology capable of regulating the morphology of a dimorphic fungal cell, and operably linked thereto

ii) a second nucleotide sequence comprising an expression signal capable of directing the expression of the first nucleotide sequence in a dimorphic fungal cell,

wherein the first and second nucleotide sequences are not natively associated, and wherein said first and/or second nucleotide sequence is derived from a microbial cell, including a microbial cell selected from the group of microbial cells consisting of eukaryotic microbial cells and procaryotic microbial cells.

When the microbial cell from which the first and/or second nucleotide sequence is derived is a eukaryotic microbial cell, it is preferably selected from the group of eukaryotic cells consisting of fungal cells and yeast cells.

In one preferred embodiment, the eukaryotic microbial cell from which the first and/or second nucleotide sequence is derived is a fungal cell, including a filamentous fungal cell, including a dimorphic fungal cell, including dimorphic fungal cells capable of growing as a multinucleated cell having a unicellular, essentially spherical morphology and/or capable of growing as a mycelium having a filamentous structure and comprising multinucleated cells, including a fungal cell belonging to the class of Zygomycetes, including a fungal cell belonging to the order of Mucorales, including a fungal cell belonging to the genus selected from the group of genera consisting of Mucor, Thermomucor, Rhizomucor, Mycotypha, Rhizopus and Cokeromyces, including Cokeromyces recurvatus, including a fungal cell belonging to the genus Mucor, including a fungal cell selected from the group of Mucor species consisting of M. circinelloides; M. hiemalis, M. rouxii, M. genevensis, M. bacilliformis, and M. subtillissimus, including a fungal cell such as M. circinelloides.

In another preferred embodiment, the eukaryotic microbial cell from which the first and/or second nucleotide sequence is derived is a dimorphic fungal cells capable of growing as a uninucleated cell having a unicellular, essentially spherical morphology, and/or capable of growing as a filamentous structure comprising uninucleated cells. Examples of such dimorphic fungi includes, but is not limited to Yarrowia, Candida and Arxula.

Vector Comprising the Polynucleotide Encoding the at least One Regulator of Morphology

The polynucleotide according to the invention comprising

i) a first nucleotide sequence encoding at least one regulator of morphology capable of regulating the morphology of a dimorphic fungal cell, and operably linked thereto

ii) a second nucleotide sequence comprising an expression signal capable of directing the expression of the first nucleotide sequence in a dimorphic fungal cell,

wherein the first and second nucleotide sequences are not natively associated, is in one embodiment located on a extrachromosomal, recombinant DNA molecule, preferably in the form of an expression vector, which may further comprise a signal sequence encoding a signal peptide, wherein the signal sequence is operably linked to the coding sequence of the first nucleotide sequence. The recombinant DNA molecule preferably further comprises a selectable marker, and optionally a genetic element, preferably a transposon, capable of mediating transposition of the recombinant DNA molecule. In another embodiment, the first and second nucleotide sequences are chromosomally located. Artificial chromosomes, including BACs and YACs, are included in the term vector as used herein.

Regulators of Morphology

A regulator of morphology as used herein includes any polypeptide the recombinant production of which in a fungal cell, including a dimorphic fungal cell, results in an altered filamentation including an increased or decreased filamentation, as compared to the filamentation observed when the at least one regulator is encoded by a polynucleotide operably linked to its native expression signal.

It is preferred that the recombinant production in a fungal cell, including a dimorphic fungal cell, of the at least one regulator of morphology results in an increased filamentation. In the case of a dimorphic fungal cell, the production of the at least one regulator of morphology generates in one preferred embodiment an increased filamentation by inducing a dimorphic shift from i) a predominant yeast-like morphology characterised in one embodiment by a multinucleated cell having a unicellular, essentially spherical morphology to ii) a predominant filamentous structure characterised by a mycelium comprising multinucleated cells.

Example 1 is a study of the morphologies associated with a dimorphic shift of Mucor circinelloides. The dimorphic fungal cell Mucor circinelloides can respond to environmental cues by growing e.g. as a multinucleated cell having a unicellular, essentially spherical morphology, or as a filamentous fungal cell characterised by a mycelium comprising multinucleated cells. Both filamentous and non-filamentous growth is organized in a single multinucleated cell. Unicellular Mucor circinelloides cells of essentially spherical morphology are multipolar meaning that daughter cells can originate at different positions of the mother cell as opposed to bipolar and monopolar cells. The Mucor circinelloides cells each harbor more than one nucleus, while Mucor circinelloides mycelium is aseptate, but with evenly distributed nuclei.

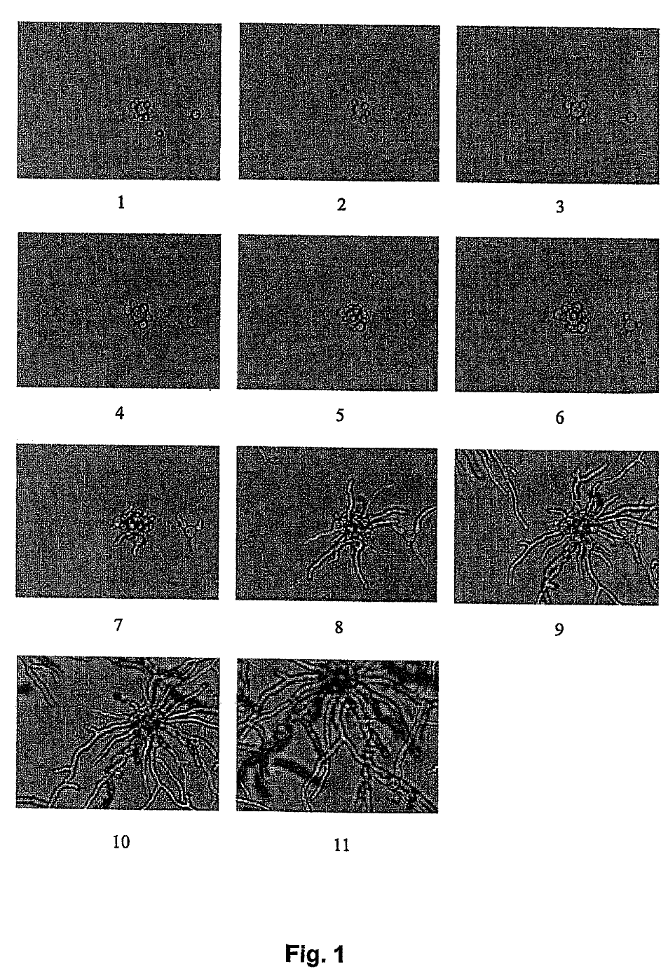

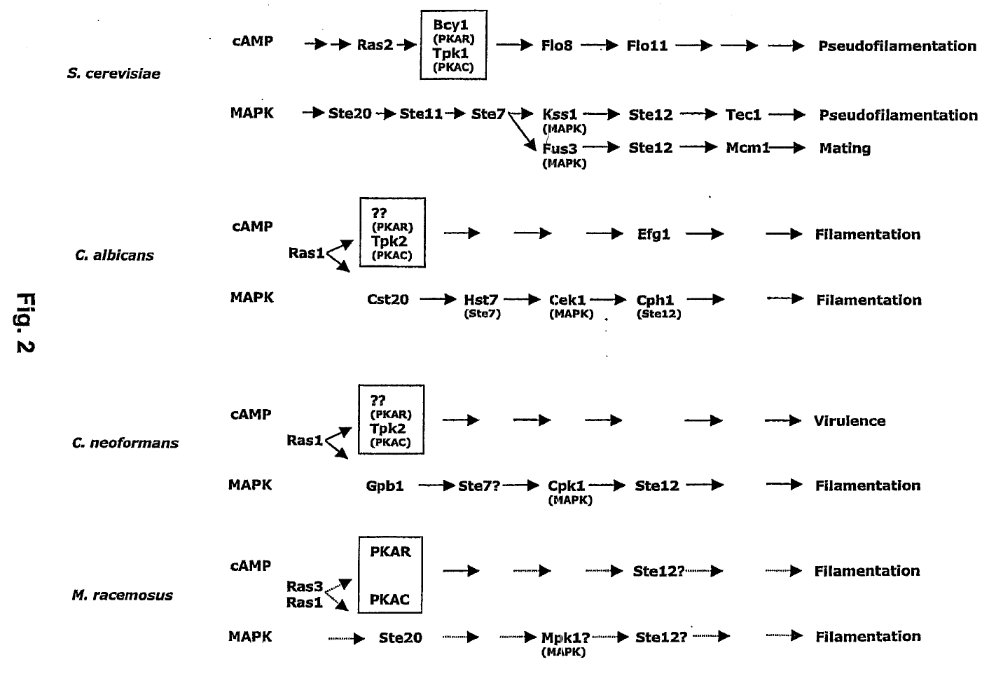

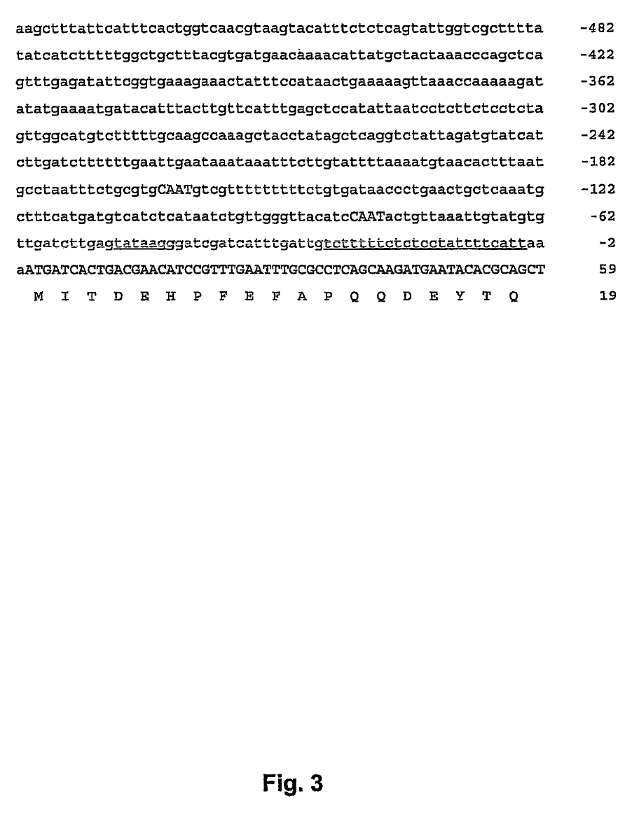

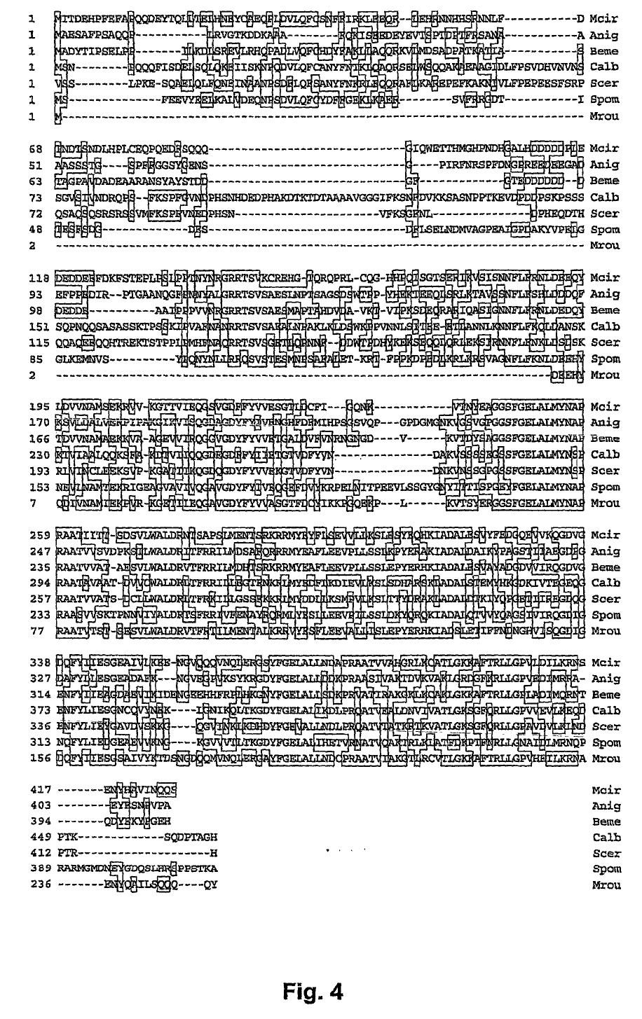

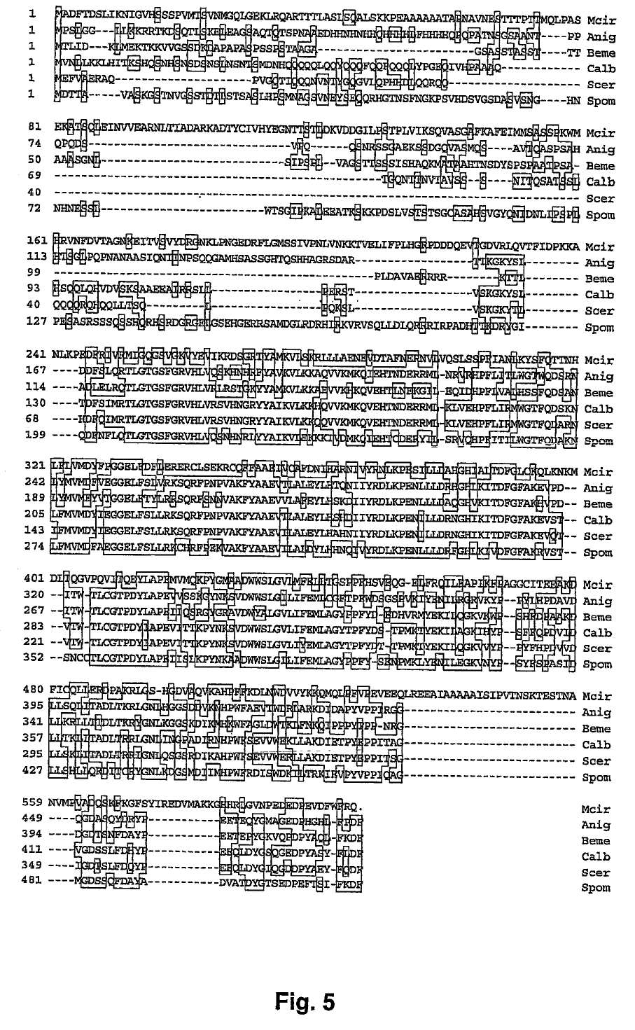

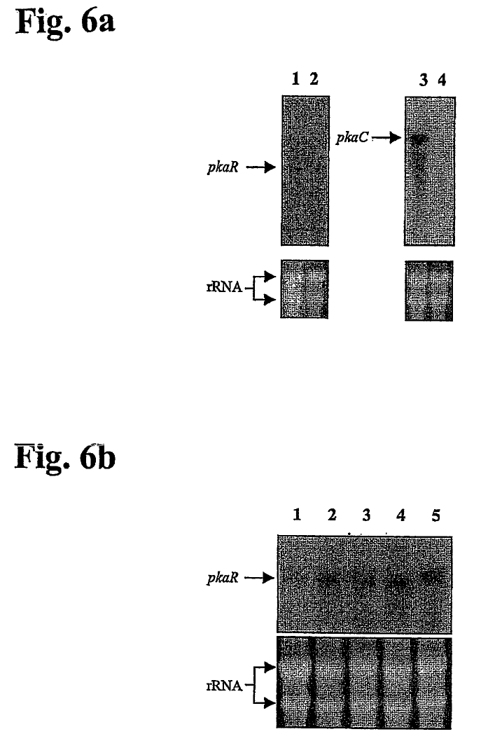

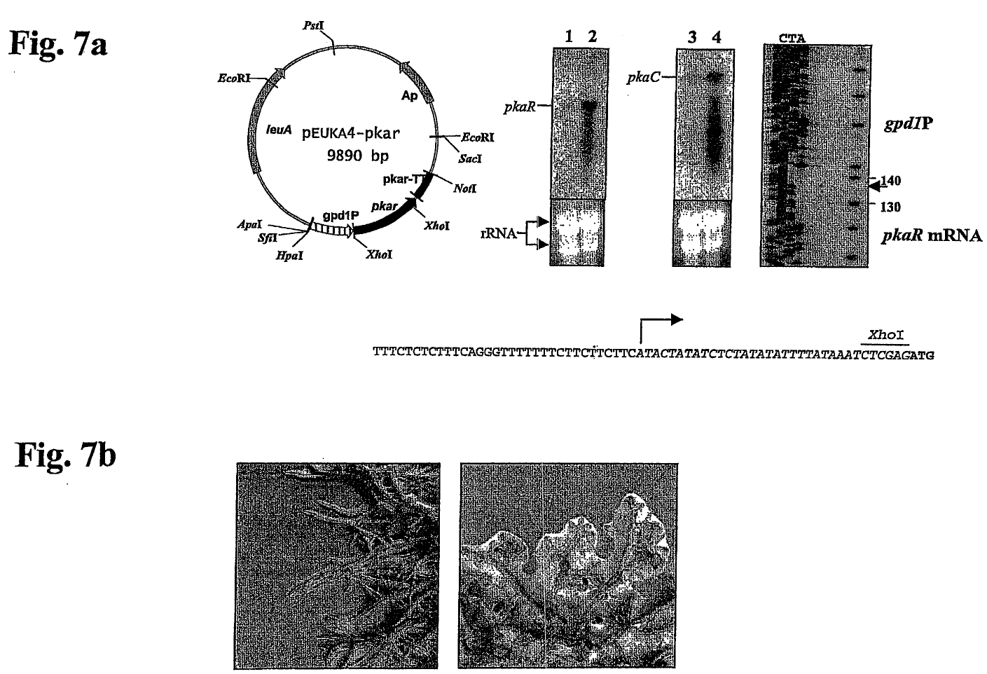

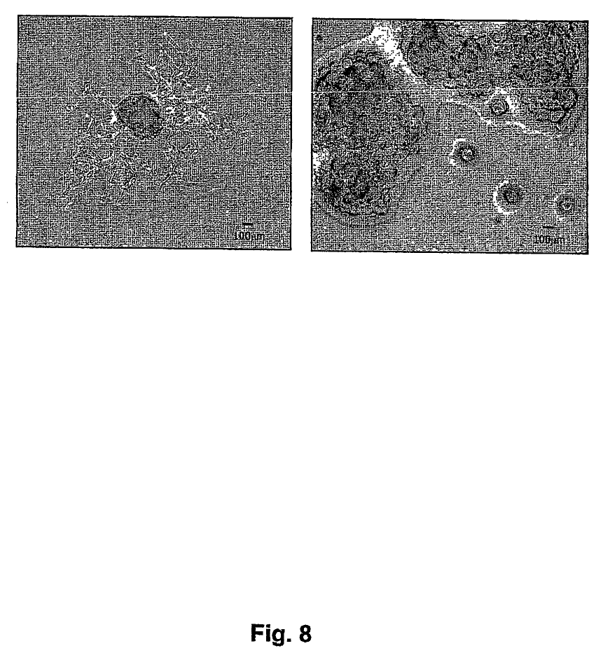

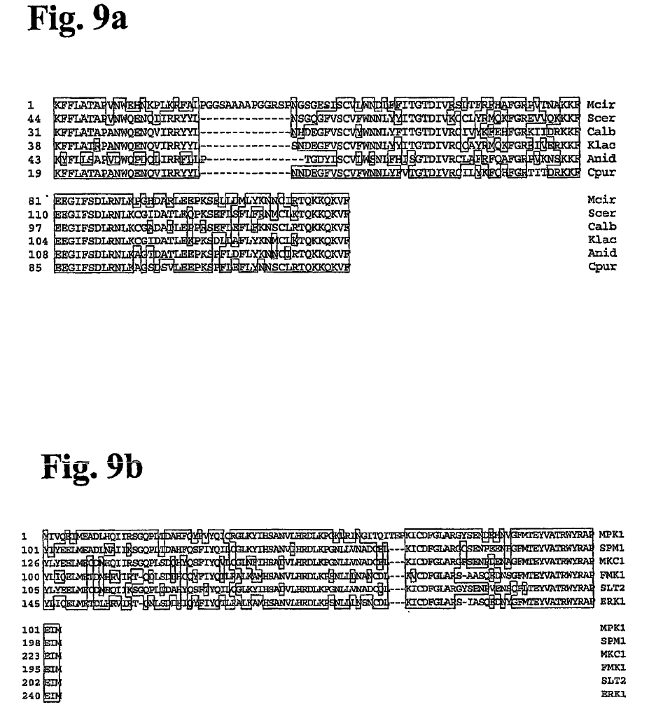

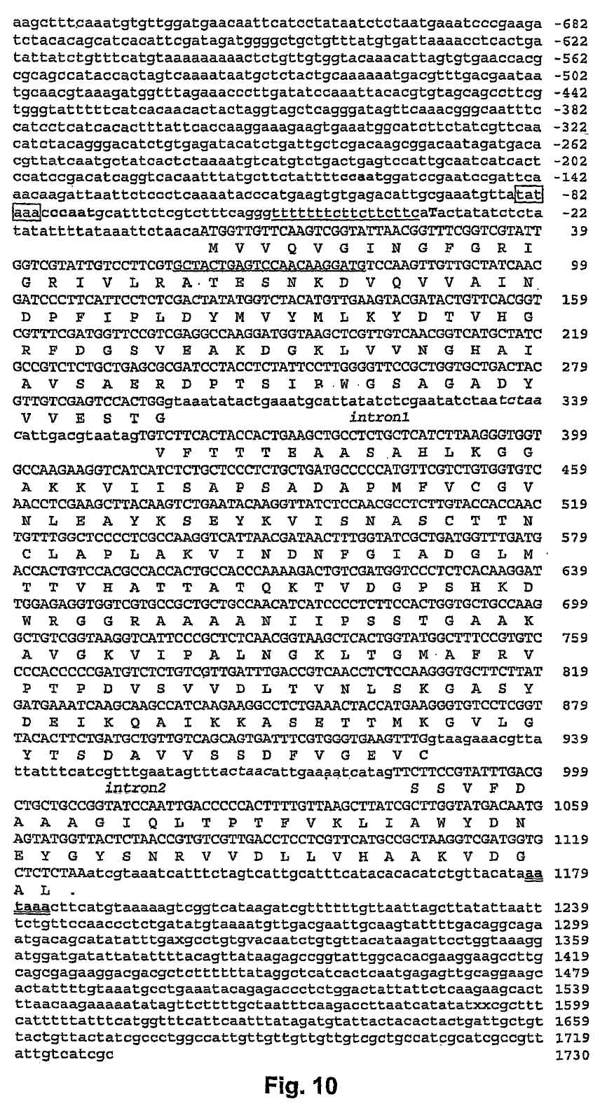

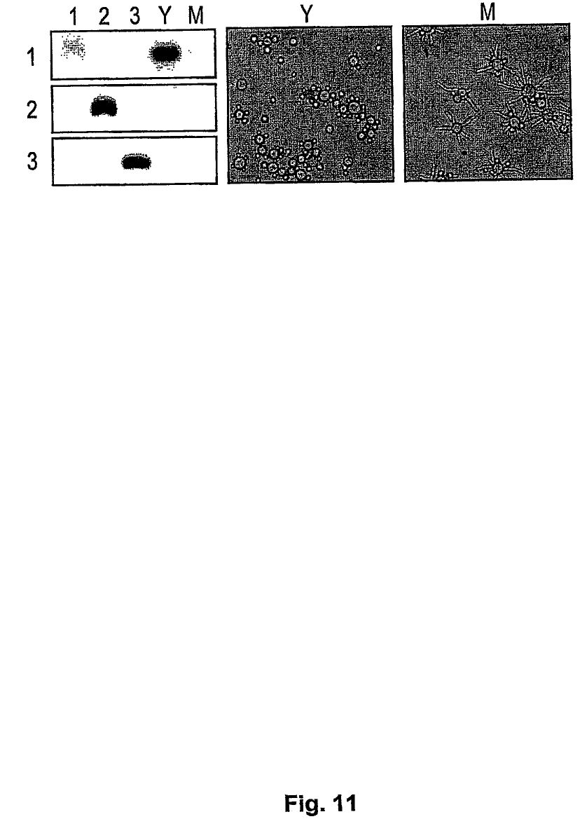

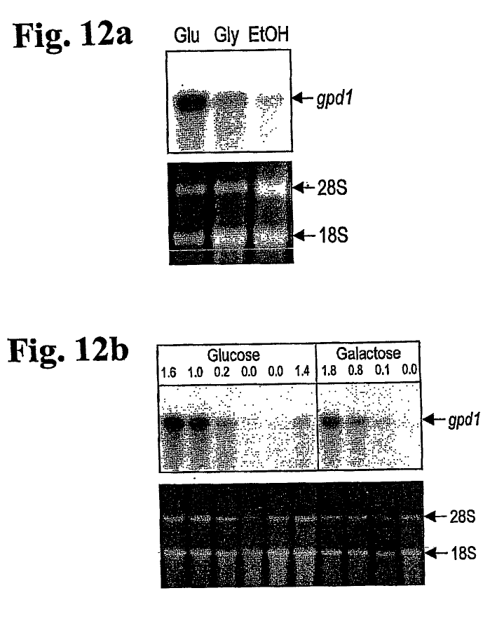

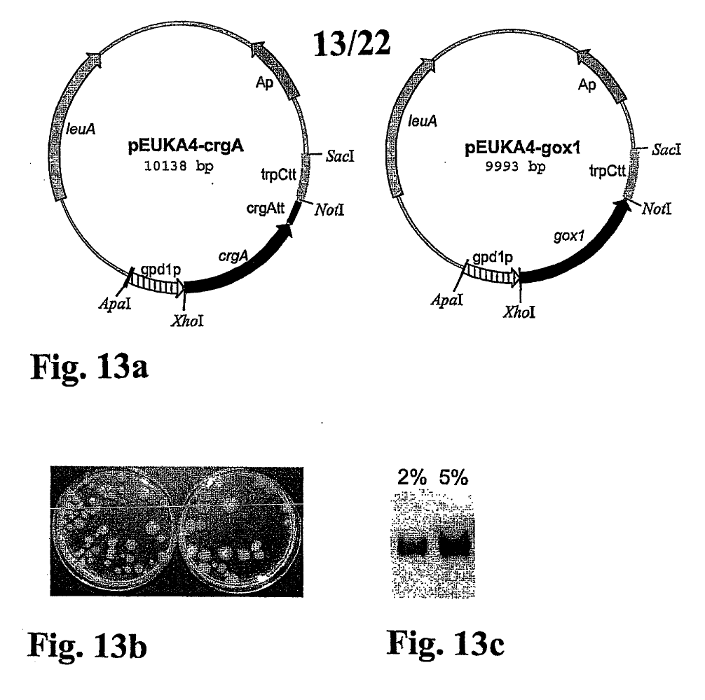

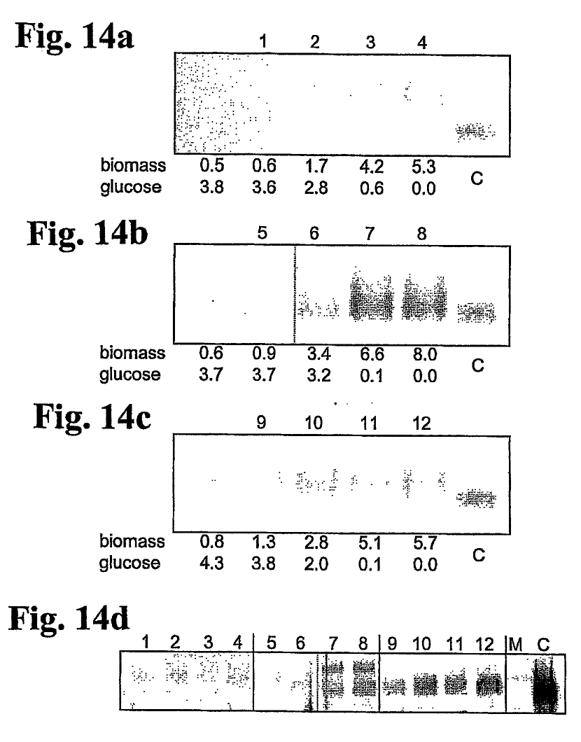

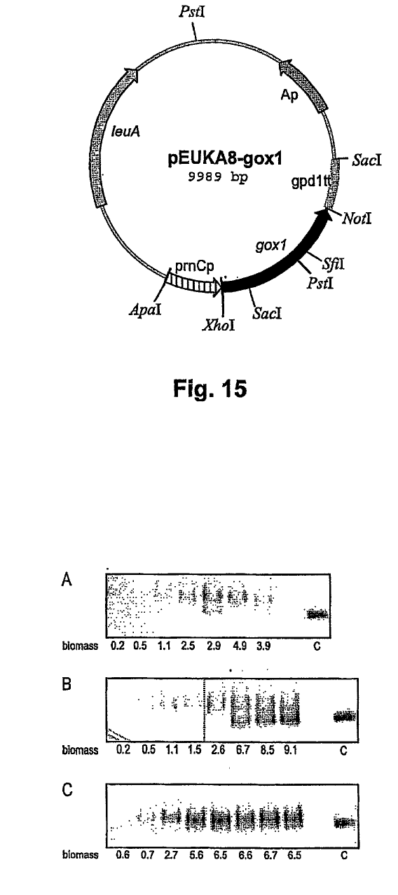

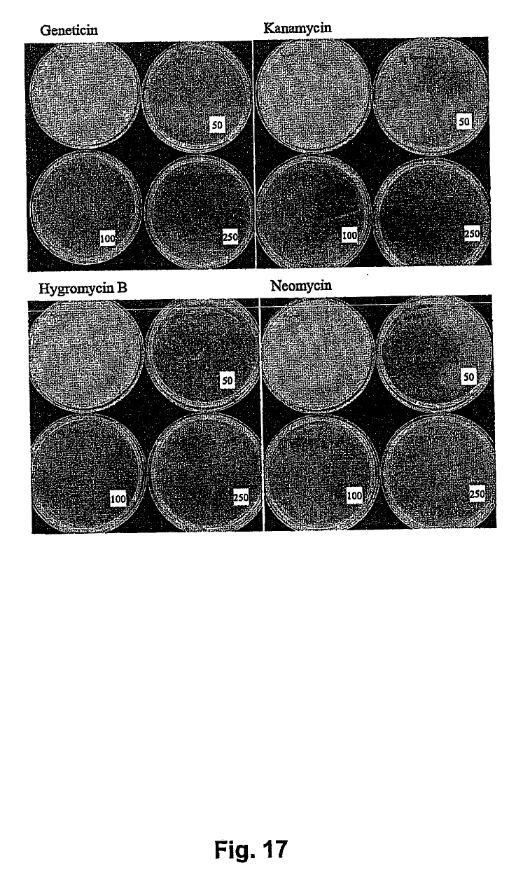

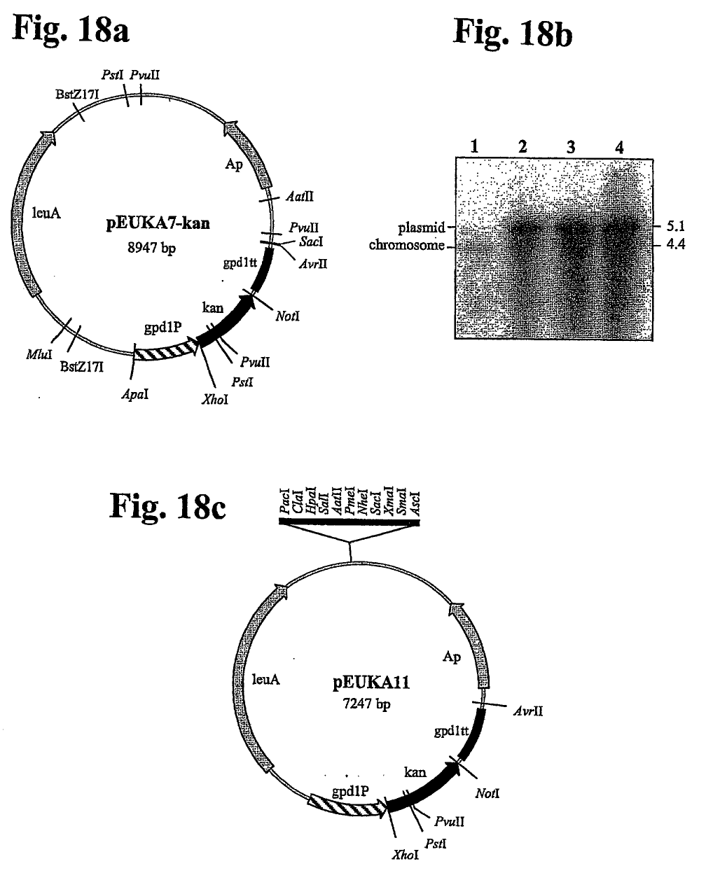

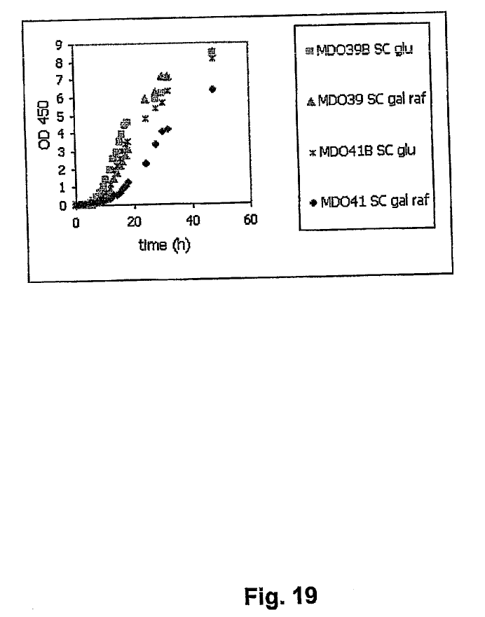

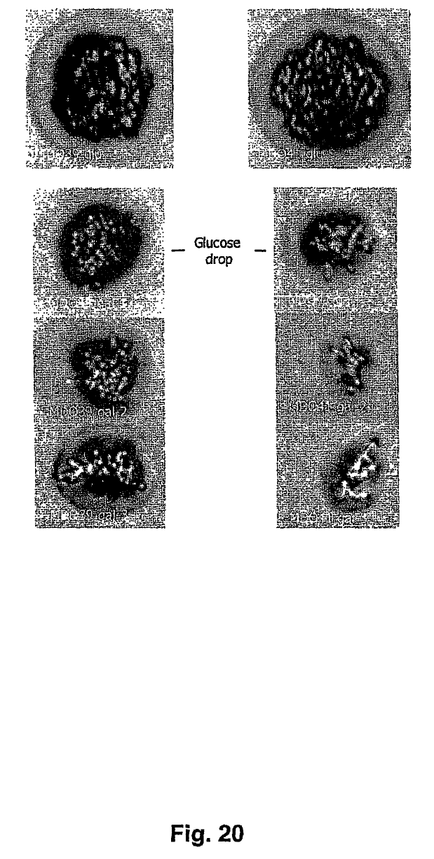

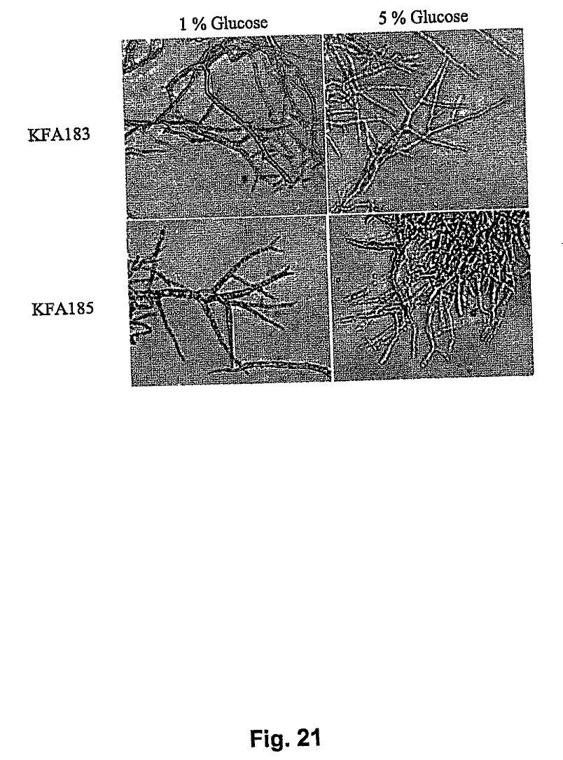

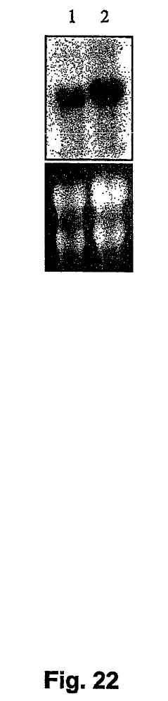

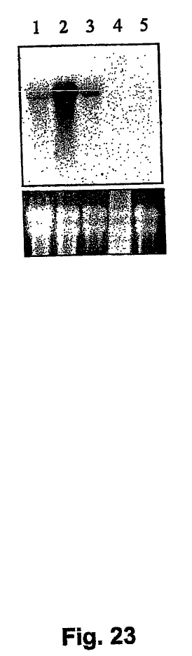

Following a shift from anaerobic to aerobic conditions, a transition from non-filamentous cell morphology to filamentous structures occurs. FIG. 1 illustrates the monitoring of this morphogenetic process on single Mucor circinelloides cells.

Preferred regulators in accordance with the present invention are kinases and transcription factors participating in the genetic networks regulating fungal cellular processes such as e.g. filamentation. A number of such regulators are illustrated in FIG. 2. Further preferred regulators of morphology according to the present invention are described herein below.

Key Components of the Signal Transduction Network Regulating Morphology

Example 2 discloses the cloning of pkaR and pkaC encoding the regulatory subunit (PKAR) and the catalytic subunit (PKAC), respectively, of the cAMP-dependent protein kinase A of M. circinelloides. In anaerobically grown yeast cells, the levels of expression of both pkaR and pkaC was significantly higher as compared to the levels of expression in aerobically grown mycelium. However, during the dimorphic shift, i.e., during the transition from anaerobic yeast growth to aerobic filamentous growth, the expression of pkaR was found to increase approximately twofold.

Induction of pkar expression is dependent of the shift from anaerobic to aerobic growth and independent of glucose concentration. Overexpression of pkaR resulted in a multi-branched colony phenotype on solid medium.

Mitogen-activated protein (MAP) kinases and the transcription factor STE12 are elements of parallel signal transduction pathways involved in filamentation in different fungi (FIG. 2). Via degenerate PCR, the present invention has identified both a gene encoding a STE12 homologue (ste12) and a gene encoding a MAP kinase homologue (mpk1; mitogen activated protein kinase 1). Further, identification of an upstream regulator, the MAP kinase kinase kinase STE20 homologue (ste20) is described in Example 2.

PKAR

Accordingly, when the present invention in one aspect relates to an isolated polynucleotide comprising

i) a first nucleotide sequence encoding at least one regulator of morphology capable of regulating the morphology of a dimorphic fungal cell, and operably linked thereto

ii) a second nucleotide sequence comprising an expression signal capable of directing the expression of the first nucleotide sequence in a dimorphic fungal cell,

wherein the first and second nucleotide sequences are not natively associated,

the first nucleotide sequence is preferably selected from the group consisting of

i) a polynucleotide comprising nucleotides 542 to 1930 of SEQ ID NO:1, and

ii) a polynucleotide comprising or essentially consisting of the coding sequence of pkaR encoding the regulatory subunit of protein kinase A (PKAR) of Mucor circinelloides, as deposited with DSMZ under accession number DSM 14062; and

iii) a polynucleotide encoding a polypeptide having the amino acid sequence as shown in SEQ ID NO:2; and

iv) a polynucleotide encoding a fragment of a polypeptide encoded by polynucleotides (i) or (ii), wherein said fragment

a) has Mucor circinelloides protein kinase A regulatory subunit activity and is a regulator of morphology of a dimorphic fungal cell; and/or

b) is recognised by an antibody, or a binding fragment thereof, which is capable of recognising the inhibitor peptide at the N-terminal end of the regulatory subunit of Mucor circinelloides protein kinase A, or a cAMP binding domain of the regulatory subunit of Mucor circinelloides protein kinase A, wherein said inhibitor peptide or said cAMP binding domain is preferably comprised by the polypeptide having the amino acid sequence as shown in SEQ ID NO:2; and/or

c) is competing with a polypeptide comprising or essentially consisting of the amino acid sequence as shown in SEQ ID NO:2 for binding to at least one predetermined binding partner, including cAMP and/or the Mucor circinelloides catalytic subunit for protein kinase A; and

v) a polynucleotide, the complementary strand of which hybridizes, under stringent conditions, with a polynucleotide as defined in any of (i), (ii) (iii), and (iv), and encodes a polypeptide that

a) has Mucor circinelloides protein kinase A regulatory subunit acitivty and is a regulator of morphology of a dimorphic fungal cell; and/or

b) is recognised by an antibody, or a binding fragment thereof, which is capable of recognising the inhibitor peptide at the N-terminal end of the regulatory subunit of Mucor circinelloides protein kinase A, or a cAMP binding domain of the regulatory subunit of Mucor circinelloides protein kinase A, wherein said inhibitor peptide or said cAMP binding domain is preferably comprised by the polypeptide having the amino acid sequence as shown in SEQ ID NO:2; and/or

c) is competing with a polypeptide comprising or essentially consisting of the amino acid sequence as shown in SEQ ID NO:2 for binding to at least one predetermined binding partner, including cAMP and/or the Mucor circinelloides catalytic subunit for protein kinase A; and

vi) a polynucleotide comprising a nucleotide sequence which is degenerate to the nucleotide sequence of a polynucleotide as defined in any of (iv) and (v),

and the complementary strand of such a polynucleotide.

Stringent conditions as used herein shall denote stringency as normally applied in connection with Southern blotting and hybridization as described e.g. by Southern E. M., 1975, J. Mol. Biol. 98:503-517. For such purposes it is routine practise to include steps of prehybridization and hybridization. Such steps are normally performed using solutions containing 6×SSPE, 5% Denhardt's, 0.5% SDS, 50% formamide, 100 μg/ml denaturated salmon testis DNA (incubation for 18 hrs at 42° C.), followed by washings with 2×SSC and 0.5% SDS (at room temperature and at 37° C.), and a washing with 0.1×SSC and 0.5% SDS (incubation at 68° C. for 30 min), as described by Sambrook et al., 1989, in “Molecular Cloning/A Laboratory Manual”, Cold Spring Harbor), which is incorporated herein by reference.

Accordingly, the first nucleotide sequence in one preferred embodiment preferably comprises nucleotides 542 to 1930 of SEQ ID NO:1.

In another embodiment, the first nucleotide sequence comprises or essentially consists of the coding sequence of pkaR encoding the regulatory subunit of protein kinase A (PKAR) of Mucor circinelloides, as deposited with DSMZ under accession number DSM 14062.

In yet another embodiment, the first nucleotide sequence encodes a polypeptide having the amino acid sequence as shown in SEQ ID NO:2.

In a further embodiment, the first nucleotide sequence encodes a fragment of the polypeptide having the amino acid sequence as shown in SEQ ID NO:2, wherein said fragment

a) has Mucor circinelloides protein kinase A regulatory subunit activity and is a regulator of morphology of a dimorphic fungal cell; and/or

b) is recognised by an antibody, or a binding fragment thereof, which is capable of recognising the inhibitor peptide at the N-terminal end of the regulatory subunit of Mucor circinelloides protein kinase A, or a cAMP binding domain of the regulatory subunit of Mucor circinelloides protein kinase A, wherein said inhibitor peptide or said cAMP binding domain is preferably comprised by the polypeptide having the amino acid sequence as shown in SEQ ID NO:2; and/or

c) is competing with a polypeptide comprising or essentially consisting of the amino acid sequence as shown in SEQ ID NO:2 for binding to at least one predetermined binding partner, including cAMP and/or the Mucor circinelloides catalytic subunit for protein kinase A; and

In a still further embodiment, the first polynucleotide sequence comprises a polynucleotide the complementary strand of which hybridizes under stringent conditions with a polypeptide that

a) has Mucor circinelloides protein kinase A regulatory subunit activity and is a regulator of morphology of a dimorphic fungal cell; and/or

b) is recognised by an antibody, or a binding fragment thereof, which is capable of recognising the inhibitor peptide at the N-terminal end of the regulatory subunit of Mucor circinelloides protein kinase A, or a cAMP binding domain of the regulatory subunit of Mucor circinelloides protein kinase A, wherein said inhibitor peptide or said cAMP binding domain is preferably comprised by the polypeptide having the amino acid sequence as shown in SEQ ID NO:2; and/or

c) is competing with a polypeptide comprising or essentially consisting of the amino acid sequence as shown in SEQ ID NO:2 for binding to at least one predetermined binding partner, including cAMP and/or the Mucor circinelloides catalytic subunit for protein kinase A.

The regulatory subunit of protein kinase A consists of an inhibitor peptide at the N-terminal end (around position 91-112) that occupies the active site of the catalytic subunit (PKAC) in the holoenzyme form of the protein, keeping the PKAC subunit from phosphorylating any substrate molecules. Two cAMP binding domains called A and B are also present in PKAR. When cAMP binds to these sites, the molecule undergoes a conformational change, withdrawing the inhibitor peptide from PKAC and allowing PKAR and PKAC to separate. At low cAMP levels, PKAR reverts to its original conformation and binds to PKAC.

Polyclonal antibodies against PKAR exist, and some bind to PKAR irrespective of its binding to PKAC. Standard cAMP measurements are also well known to the skilled person.

There is also provided a polynucleotide comprising a first nucleotide sequence which is degenerate to any of the sequences described herein immediately above, as well as a polynucleotide comprising a first nucleotide sequence in the form of the the complementary strand of polynucleotides described herein immediately above.

PKAC

Accordingly, when the present invention in one aspect relates to an isolated polynucleotide comprising

i) a first nucleotide sequence encoding at least one regulator of morphology capable of regulating the morphology of a dimorphic fungal cell, and operably linked thereto

ii) a second nucleotide sequence comprising an expression signal capable of directing the expression of the first nucleotide sequence in a dimorphic fungal cell,

wherein the first and second nucleotide sequences are not natively associated,

the first nucleotide sequence is preferably selected from the group consisting of

i) a polynucleotide comprising nucleotides 534 to 2471 of SEQ ID NO:11, and

ii) a polynucleotide comprising or essentially consisting of the coding sequence of pkaC encoding the catalytic subunit of protein kinase A (PKAC) of Mucor circinelloides, as deposited with DSMZ under accession number DSM 14839; and

iii) a polynucleotide encoding a polypeptide having the amino acid sequence as shown in SEQ ID NO:12; and

iv) a polynucleotide encoding a fragment of a polypeptide encoded by polynucleotides (i) or (ii), wherein said fragment

a) has Mucor circinelloides catalytic subunit of protein kinase A activity and is a regulator of morphology of a dimorphic fungal cell; and/or

b) is recognised by an antibody, or a binding fragment thereof, which is capable of recognising a protein kinase A binding domain of Mucor circinelloides PKAC, wherein said domain is comprised by the polypeptide having the amino acid sequence as shown in SEQ ID NO:12; and/or

c) is competing with a polypeptide comprising or essentially consisting of the amino acid sequence as shown in SEQ ID NO:12 for binding to at least one predetermined binding partner, including PKAR; and

v) a polynucleotide, the complementary strand of which hybridizes, under stringent conditions, with a polynucleotide as defined in any of (i), (ii) (iii), and (iv), and encodes a polypeptide that

a) has Mucor circinelloides catalytic subunit of protein kinase A activity and is a regulator of morphology of a dimorphic fungal cell; and/or

b) is recognised by an antibody, or a binding fragment thereof, which is capable of recognising a protein kinase A binding domain of Mucor circinelloides PKAC, wherein said domain is comprised by the polypeptide having the amino acid sequence as shown in SEQ ID NO:12; and/or

c) is competing with a polypeptide comprising or essentially consisting of the amino acid sequence as shown in SEQ ID NO:12 for binding to at least one predetermined binding partner, including PKAR; and

vi) a polynucleotide comprising a nucleotide sequence which is degenerate to the nucleotide sequence of a polynucleotide as defined in any of (iv) and (v),

and the complementary strand of such a polynucleotide.

Accordingly, the first nucleotide sequence in one preferred embodiment preferably comprises nucleotides 534 to 2471 of SEQ ID NO:11.

In another embodiment, the first nucleotide sequence comprises or essentially consists of the coding sequence of pkaC encoding the catalytic subunit of protein kinase A (PKAC) of Mucor circinelloides, as deposited with DSMZ under accession number DSM 14839.

In yet another embodiment, the first nucleotide sequence encodes a polypeptide having the amino acid sequence as shown in SEQ ID NO:12.

In a further embodiment, the first nucleotide sequence encodes a fragment of the polypeptide having the amino acid sequence as shown in SEQ ID NO:12, wherein said fragment

a) has Mucor circinelloides catalytic subunit of protein kinase A activity and is a regulator of morphology of a dimorphic fungal cell; and/or

b) is recognised by an antibody, or a binding fragment thereof, which is capable of recognising a protein kinase A binding domain of Mucor circinelloides PKAC, wherein said domain is comprised by the polypeptide having the amino acid sequence as shown in SEQ ID NO:12; and/or

c) is competing with a polypeptide comprising or essentially consisting of the amino acid sequence as shown in SEQ ID NO:12 for binding to at least one predetermined binding partner, including PKAR.

In a still further embodiment, the first polynucleotide sequence comprises a polynucleotide the complementary strand of which hybridizes under stringent conditions with a polypeptide that

a) has Mucor circinelloides catalytic subunit of protein kinase A activity and is a regulator of morphology of a dimorphic fungal cell; and/or

b) is recognised by an antibody, or a binding fragment thereof, which is capable of recognising a protein kinase A binding domain of Mucor circinelloides PKAC, wherein said domain is comprised by the polypeptide having the amino acid sequence as shown in SEQ ID NO:12; and/or

c) is competing with a polypeptide comprising or essentially consisting of the amino acid sequence as shown in SEQ ID NO:12 for binding to at least one predetermined binding partner, including PKAR.

There is also provided a polynucleotide comprising a first nucleotide sequence which is degenerate to any of the sequences described herein immediately above, as well as a polynucleotide comprising a first nucleotide sequence in the form of the the complementary strand of polynucleotides described herein immediately above.

STE20

STE20 is an upstream regulator in the MAP kinase transduction pathway. When the present invention in one aspect relates to an isolated polynucleotide comprising

i) a first nucleotide sequence encoding at least one regulator of morphology capable of regulating the morphology of a dimorphic fungal cell, and operably linked thereto

ii) a second nucleotide sequence comprising an expression signal capable of directing the expression of the first nucleotide sequence in a dimorphic fungal cell,

wherein the first and second nucleotide sequences are not natively associated,

the first nucleotide sequence is preferably selected from the group consisting of

i) a polynucleotide comprising nucleotides 1 to 634 of SEQ ID NO:3, and

ii) a polynucleotide comprising or essentially consisting of the coding sequence of ste20 encoding a MAP kinase kinase kinase (STE20) of Mucor circinelloides, as deposited with DSMZ under accession number DSM 14065; and

iii) a polynucleotide encoding a polypeptide having the amino acid sequence as shown in SEQ ID NO:4; and

iv) a polynucleotide encoding a fragment of a polypeptide encoded by polynucleotides (i) or (ii), wherein said fragment

a) has Mucor circinelloides STE20 activity and is a regulator of morphology of a dimorphic fungal cell; and/or

b) is recognised by an antibody, or a binding fragment thereof, which is capable of recognising the catalytic domain of Mucor circinelloides STE20, wherein said catalytic domain is comprised by the polypeptide having the amino acid sequence as shown in SEQ ID NO:4; and/or

c) is competing with a polypeptide comprising or essentially consisting of the amino acid sequence as shown in SEQ ID NO:4 for interaction with a substrate for phosphorylation, including triphosphate nucleotides, including ATP; and

v) a polynucleotide, the complementary strand of which hybridizes, under stringent conditions, with a polynucleotide as defined in any of (i), (ii) (iii), and (iv), and encodes a polypeptide that

a) has Mucor circinelloides STE20 activity and is a regulator of morphology of a dimorphic fungal cell; and/or

b) is recognised by an antibody, or a binding fragment thereof, which is capable of recognising the catalytic domain of Mucor circinelloides STE20, wherein said catalytic domain is comprised by the polypeptide having the amino acid sequence as shown in SEQ ID NO:4; and/or

c) is competing with a polypeptide comprising or essentially consisting of the amino acid sequence as shown in SEQ ID NO:4 for interaction with a substrate for phosphorylation, including triphosphate nucleotides, including ATP; and

vi) a polynucleotide comprising a nucleotide sequence which is degenerate to the nucleotide sequence of a polynucleotide as defined in any of (iv) and (v),

and the complementary strand of such a polynucleotide.

Accordingly, the first nucleotide sequence in one preferred embodiment preferably comprises nucleotides 1 to 634 of SEQ ID NO:3.

In another embodiment, the first nucleotide sequence comprises or essentially consists of the coding sequence of ste20 encoding a MAP kinase kinase kinase (STE20) of Mucor circinelloides, as deposited with DSMZ under accession number DSM 14065.

In yet another embodiment, the first nucleotide sequence encodes a polypeptide having the amino acid sequence as shown in SEQ ID NO:4.

In a further embodiment, the first nucleotide sequence encodes a fragment of the polypeptide having the amino acid sequence as shown in SEQ ID NO:4, wherein said fragment

a) has Mucor circinelloides STE20 acitivty and is a regulator of morphology of a dimorphic fungal cell; and/or

b) is recognised by an antibody, or a binding fragment thereof, which is capable of recognising the catalytic domain of Mucor circinelloides STE20, wherein said catalytic domain is comprised by the polypeptide having the amino acid sequence as shown in SEQ ID NO:4; and/or

c) is competing with a polypeptide comprising or essentially consisting of the amino acid sequence as shown in SEQ ID NO:4 for interaction with a substrate for phosphorylation, including triphosphate nucleotides, including ATP.

In a still further embodiment, the first polynucleotide sequence comprises a polynucleotide the complementary strand of which hybridizes under stringent conditions with a polypeptide that

a) has Mucor circinelloides STE20 activity and is a regulator of morphology of a dimorphic fungal cell; and/or

b) is recognised by an antibody, or a binding fragment thereof, which is capable of recognising the catalytic domain of Mucor circinelloides STE20, wherein said catalytic domain is comprised by the polypeptide having the amino acid sequence as shown in SEQ ID NO:4; and/or

c) is competing with a polypeptide comprising or essentially consisting of the amino acid sequence as shown in SEQ ID NO:4 for interaction with a substrate for phosphorylation, including triphosphate nucleotides, including ATP.

There is also provided a polynucleotide comprising a first nucleotide sequence which is degenerate to any of the sequences described herein immediately above, as well as a polynucleotide comprising a first nucleotide sequence in the form of the the complementary strand of polynucleotides described herein immediately above.

Besides being capable of recognising the catalytic domain of Mucor circinelloides STE20, or a fragment thereof, the antibody, or a binding fragment thereof, may also recognise other motifs present in the primary amino acid sequence of STE20. Examples of such motifs are listed herein below:

Many putative downstream effectors of the small GTPases Cdc42 and Rac contain a GTPase binding domain (GBD), also called p21 binding domain (PBD), which has been shown to specifically bind the GTP bound form of Cdc42 or Rac, with a preference for Cdc42. The most conserved region of GBD/PBD domains is the N-terminal Cdc42/Rac interactive binding motif (CRIB), which consists of about 16 amino acids with the consensus sequence I-S-X-P-X(2,4)-F-X-H-X(2)-H-V-G. Although the CRIB motif is necessary for the binding to Cdc42 and Rac, it is not sufficient to give high-affinity binding.

A less well conserved inhibitory switch (IS) domain responsible for maintaining the proteins in a basal (autoinhibited) state is located C-terminaly of the CRIB-motif. GBD domains can adopt related but distinct folds depending on context. Although GBD domains are largely unstructured in the free state, the IS domain forms an N-terminal beta hairpin that immediately follows the conserved CRIB motif and a central bundle of three alpha helices in the autoinhibited state. The interaction between GBD domains and their respective G proteins leads to the formation of a high-affinity complex in which unstructured regions of both the effector and the G protein become rigid. CRIB motifs from various GBD domains interact with Cdc42 in a similar manner, forming an intermolecular beta-sheet with strand beta-2 of Cdc42. Outside the CRIB motif, the C-termini of the various GBD domains are very divergent and show variation in their mode of binding to Cdc42, perhaps determining the specificity of the interaction. Binding of Cdc42 or Rac to the GBD domain causes a dramatic conformational change, refolding part of the IS domain and unfolding the rest.

Proteins known to contain a GBD domain include mammalian activated Cdc42-associated kinases (ACKs), nonreceptor tyrosine kinases implicated in integrin-coupled pathways, mammalian p21-activated kinases (PAK1 to PAK4), serine/threonine kinases that modulate cytoskeletal assembly and activate MAP-kinase pathways and yeast STE20, homologue of mammalian PAKs. STE20 is involved in the mating/pheromone MAP kinase cascade.

MPK1

Mitogen-activated protein kinases (MAP kinases) form a group of serine/threonine protein kinases that play important roles in signal transduction pathways regulating adaptative response to a wide range of stimuli.

When the present invention in one aspect relates to an isolated polynucleotide comprising

i) a first nucleotide sequence encoding at least one regulator of morphology capable of regulating the morphology of a dimorphic fungal cell, and operably linked thereto

ii) a second nucleotide sequence comprising an expression signal capable of directing the expression of the first nucleotide sequence in a dimorphic fungal cell,

wherein the first and second nucleotide sequences are not natively associated,

the first nucleotide sequence is preferably selected from the group consisting of

i) a polynucleotide comprising nucleotides 1 to 541 of SEQ ID NO:5, and

ii) a polynucleotide comprising or essentially consisting of the coding sequence of mpk1 encoding mitogen activated protein kinase 1 of Mucor circinelloides, as deposited with DSMZ under accession number DSM 14063 and

iii) a polynucleotide encoding a polypeptide having the amino acid sequence as shown in SEQ ID NO:6; and

iv) a polynucleotide encoding a fragment of a polypeptide encoded by polynucleotides (i) or (ii), wherein said fragment

a) has Mucor circinelloides mitogen activated protein kinase 1 activity and is a regulator of morphology of a dimorphic fungal cell; and/or

b) is recognised by an antibody, or a binding fragment thereof, which is capable of recognising the catalytic domain of Mucor circinelloides mitogen activated protein kinase 1, wherein said catalytic domain is comprised by the polypeptide having the amino acid sequence as shown in SEQ ID NO:6; and/or

c) is competing with a polypeptide comprising or essentially consisting of the amino acid sequence as shown in SEQ ID NO:6 for interaction with a substrate for phosphorylation, including triphosphate nucleotides, including ATP; and

v) a polynucleotide, the complementary strand of which hybridizes, under stringent conditions, with a polynucleotide as defined in any of (i), (ii), (iii), and (iv), said polynucleotide encoding a polypeptide having the amino acid sequence as shown in SEQ ID NO:6, or a fragment thereof, wherein said fragment

a) has Mucor circinelloides mitogen activated protein kinase 1 activity and is a regulator of morphology of a dimorphic fungal cell; and/or

b) is recognised by an antibody, or a binding fragment thereof, which is capable of recognising the catalytic domain of Mucor circinelloides mitogen activated protein kinase 1, wherein said catalytic domain is comprised by the polypeptide having the amino acid sequence as shown in SEQ ID NO:6; and/or

c) is competing with a polypeptide comprising or essentially consisting of the amino acid sequence as shown in SEQ ID NO:6 for interaction with a substrate for phosphorylation, including triphosphate nucleotides, including ATP; and

a polynucleotide comprising a nucleotide sequence which is degenerate to the nucleotide sequence of a polynucleotide as defined in any of (iv) and (v),

and the complementary strand of such a polynucleotide.

Accordingly, the first nucleotide sequence in one preferred embodiment preferably comprises nucleotides 1 to 541 of SEQ ID NO:5.

In another embodiment, the first nucleotide sequence comprises or essentially consists of the coding sequence of mpk1 encoding mitogen activated protein kinase 1 of Mucor circinelloides, as deposited with DSMZ under accession number DSM 14063.

In yet another embodiment, the first nucleotide sequence encodes a polypeptide having the amino acid sequence as shown in SEQ ID NO:6.

In a further embodiment, the first nucleotide sequence encodes a fragment of the polypeptide having the amino acid sequence as shown in SEQ ID NO:6, wherein said fragment

a) has Mucor circinelloides mitogen activated protein kinase 1 activity and is a regulator of morphology of a dimorphic fungal cell; and/or

b) is recognised by an antibody, or a binding fragment thereof, which is capable of recognising the catalytic domain of Mucor circinelloides mitogen activated protein kinase 1, wherein said catalytic domain is comprised by the polypeptide having the amino acid sequence as shown in SEQ ID NO:6; and/or

c) is competing with a polypeptide comprising or essentially consisting of the amino acid sequence as shown in SEQ ID NO:6 for interaction with a substrate for phosphorylation, including triphosphate nucleotides, including ATP; and

In a still further embodiment, the first polynucleotide sequence comprises a polynucleotide the complementary strand of which hybridizes under stringent conditions with a polypeptide that

a) has Mucor circinelloides mitogen activated protein kinase 1 activity and is a regulator of morphology of a dimorphic fungal cell; and/or

b) is recognised by an antibody, or a binding fragment thereof, which is capable of recognising the catalytic domain of Mucor circinelloides mitogen activated protein kinase 1, wherein said catalytic domain is comprised by the polypeptide having the amino acid sequence as shown in SEQ ID NO:6; and/or

c) is competing with a polypeptide comprising or essentially consisting of the amino acid sequence as shown in SEQ ID NO:6 for interaction with a substrate for phosphorylation, including triphosphate nucleotides, including ATP; and

The term catalytic domain as used herein above shall include the conserved TXY motif in which both the threonine and tyrosine residues are phosphorylated during activation of the enzyme by upstream dual-specificity MAP kinase kinases (MAPKKs). In addition to the TXY motif, other motifs include the region located just after the TXY motif and containing a F and a C residue that are MAPK-specific. The R and E residues in the first part of the pattern, and the R, D and K residues in the second part, are shared by many additional protein kinases. They have been included in the pattern to eliminate matches from unrelated sequences in the database, and to “anchor” the MAPK-specific F and C residues to this region. Accordingly, one preferred catalytic domain comprises the consensus pattern: F-x(10)-R-E-x(72,86)-R-D-x-K-x(9)-C, and this domain is preferably recognised by an antibody used to define fragments of MAPK in accordance with the present invention.

There is also provided a polynucleotide comprising a first nucleotide sequence which is degenerate to any of the sequences described herein immediately above, as well as a polynucleotide comprising a first nucleotide sequence in the form of the the complementary strand of polynucleotides described herein immediately above.

STE12

When the present invention in one aspect relates to an isolated polynucleotide comprising

i) a first nucleotide sequence encoding at least one regulator of morphology capable of regulating the morphology of a dimorphic fungal cell, and operably linked thereto

ii) a second nucleotide sequence comprising an expression signal capable of directing the expression of the first nucleotide sequence in a dimorphic fungal cell,

wherein the first and second nucleotide sequences are not natively associated,

the first nucleotide sequence is preferably selected from the group consisting of

i) a polynucleotide comprising nucleotides 1 to 384 of SEQ ID NO:7, and

ii) a polynucleotide comprising or essentially consisting of the coding sequence of ste12 encoding a transcription factor of Mucor circinelloides, as deposited with DSMZ under accession number DSM 14064; and

iii) a polynucleotide encoding a polypeptide having the amino acid sequence as shown in SEQ ID NO:8; and

iv) a polynucleotide encoding a fragment of a polypeptide encoded by polynucleotides (i) or (ii), wherein said fragment

a) has Mucor circinelloides STE12 activity and is a regulator of morphology of a dimorphic fungal cell; and/or

b) is recognised by an antibody, or a binding fragment thereof, which is i) capable of recognising an N-terminal region of STE12 involved in polynucleotide binding and/or ii) capable of recognising an induction domain located in the central region of STE12 and/or iii) capable of recognising a C-terminal region of STE12 involved in transcriptional activation, wherein said recognised region or domain is comprised by the polypeptide having the amino acid sequence as shown in SEQ ID NO:8; and/or

c) is competing with a polypeptide having the amino acid sequence as shown in SEQ ID NO:8 for binding to a predetermined binding partner, including a polynucleotide having an affinity for said polypeptide.

v) a polynucleotide, the complementary strand of which hybridizes, under stringent conditions, with a polynucleotide as defined in any of (i), (ii), (iii), and (iv), said polynucleotide encoding a polypeptide having the amino acid sequence as shown in SEQ ID NO:8, or a fragment thereof, wherein said fragment

a) has Mucor circinelloides STE12 activity and is a regulator of morphology of a dimorphic fungal cell; and/or

b) is recognised by an antibody, or a binding fragment thereof, which is i) capable of recognising an N-terminal region of STE12 involved in polynucleotide binding and/or ii) capable of recognising an induction domain located in the central region of STE12 and/or iii) capable of recognising a C-terminal region of STE12 involved in transcriptional activation, wherein said recognised region or domain is comprised by the polypeptide having the amino acid sequence as shown in SEQ ID NO:8; and/or

c) is competing with a polypeptide having the amino acid sequence as shown in SEQ ID NO:8 for binding to a predetermined binding partner, including a polynucleotide having an affinity for said polypeptide, and

vi) a polynucleotide comprising a nucleotide sequence which is degenerate to the nucleotide sequence of a polynucleotide as defined in any of (iv) and (v),

and the complementary strand of such a polynucleotide.

Accordingly, the first nucleotide sequence in one preferred embodiment preferably comprises nucleotides 1 to 384 of SEQ ID NO:7.

In another embodiment, the first nucleotide sequence comprises or essentially consists of the coding sequence of ste12 encoding a transcription factor of Mucor circinelloides, as deposited with DSMZ under accession number DSM 14064; and.

In yet another embodiment, the first nucleotide sequence encodes a polypeptide having the amino acid sequence as shown in SEQ ID NO:8.

In a further embodiment, the first nucleotide sequence encodes a fragment of the polypeptide having the amino acid sequence as shown in SEQ ID NO:8, wherein said fragment

a) has Mucor circinelloides STE12 activity and is a regulator of morphology of a dimorphic fungal cell; and/or

b) is recognised by an antibody, or a binding fragment thereof, which is i) capable of recognising an N-terminal region of STE12 involved in polynucleotide binding and/or ii) capable of recognising an induction domain located in the central region of STE12 and/or iii) capable of recognising a C-terminal region of STE12 involved in transcriptional activation, wherein said recognised region or domain is comprised by the polypeptide having the amino acid sequence as shown in SEQ ID NO:8; and/or

c) is competing with a polypeptide having the amino acid sequence as shown in SEQ ID NO:8 for binding to a predetermined binding partner, including a polynucleotide having an affinity for said polypeptide.

In a still further embodiment, the first polynucleotide sequence comprises a polynucleotide the complementary strand of which hybridizes under stringent conditions with a polypeptide that

a) has Mucor circinelloides STE12 activity and is a regulator of morphology of a dimorphic fungal cell; and/or

b) is recognised by an antibody, or a binding fragment thereof, which is i) capable of recognising an N-terminal region of STE12 involved in polynucleotide binding and/or ii) capable of recognising an induction domain located in the central region of STE12 and/or iii) capable of recognising a C-terminal region of STE12 involved in transcriptional activation, wherein said recognised region or domain is comprised by the polypeptide having the amino acid sequence as shown in SEQ ID NO:8; and/or

c) is competing with a polypeptide having the amino acid sequence as shown in SEQ ID NO:8 for binding to a predetermined binding partner, including a polynucleotide having an affinity for said polypeptide.

There is also provided a polynucleotide comprising a first nucleotide sequence which is degenerate to any of the sequences described herein immediately above, as well as a polynucleotide comprising a first nucleotide sequence in the form of the the complementary strand of polynucleotides described herein immediately above.

The STE12 transcription factor consists of a N-terminal region involved in DNA binding (˜70% homology between fungal homologues in this region), an induction domain located in the central region of the protein and a C-terminal region that is involved in transcriptional activation.

In yeast, STE12 binds to the pheromone-responsive element found in the upstream region of many genes inducible by the mating pheromone α- and a-factor (Pi et al., 1997). A conserved 6 aa sequence (induction domain) is found among cloned fungal STE12 homologues (position 305 to 310 in the yeast protein). The DNA binding region shows at least two aa streches which are highly conserved (FFLATA and TQKKQKVF; Yue et al. 1999).

The STE12 ‘conserved’ structural motif (only four STE12 homologues are cloned to date including the Mucor circinelloides counterpart) is shown below. In one preferred embodiment, such a conserved structural motif is recognised by the antibody used to define fragments of STE12 in accordance with the present invention.

43 ISCVLWNDLFFITGTDIVRSLTFRFHAFGRPVTNAKKFEEGIFSDLRNLKPGHDARLEEP

102

103 KSELLDMLYKNNCIRTQKKQKVFFWF 128 (SEQ ID NO:8, pos. 43-128)

The term motif shall also relate to any part of the above sequence comprising any ten (10) consequtive amino acid fragments of the sequence.

Expression Signals Capable of being Regulated During Growth of a Dimorphic Fungal Cell

Expression signals as used herein preferably comprise a promoter element including a promoter sequence. As used herein, a promotor sequence is a DNA sequence which is recognized by the particular filamentous fungus for expression purposes. It is operably linked to a DNA sequence encoding the above defined polypeptides. Such linkage comprises positioning of the promoter with respect to the initiation codon of the DNA sequence encoding the signal sequence of the disclosed transformation vectors. The promoter sequence contains transcription and translation control sequences which mediate the expression of the signal sequence and heterologous polypeptide.

The promoter may be any DNA sequence that shows strong and/or regulated transcriptional activity in these species, and may be derived from genes encoding both extracellular and intracellular proteins, such as glucoamylases and glycolytic enzymes. The promoter may be either a heterologous promoter or a homologous promoter, i.e., the promoter for a gene that is either non-native or native, respectively, to the host strain being used. Useful promoters according to the present invention are e.g. the gpd1 promoter and the pmC promoter from M. circinelloides.

gpd1 encodes glyceraldehyde-3-phosphate dehydrogenase (GPD) in Mucor circinelloides, and transcription of gpd1 is detectable during vegetative growth under both aerobic and anaerobic conditions. The transcription of gpd1 in M. circinelloides is significantly higher on fermentable carbon sources than on non-fermentable carbon sources during growth under aerobic conditions, indicating that gpd1 expression is subjected to carbon catabolite regulation. A direct correlation can be established between the abundance of gpd1 mRNA and the concentration of sugar in the medium during growth.

The promoter sequence may in one embodiment be provided with linkers for the purpose of introducing specific restriction sites facilitating ligation of the promoter sequence with the gene of choice or with a selected signal peptide. Terminators and polyadenylation sequences may also be derived from the same sources as the promoters. Enhancer and regulatory sequences may also be inserted into the construct.

The promoters and enhancers that control the transcription of protein-encoding genes are composed of multiple genetic elements. The cellular machinery is able to gather and integrate the regulatory information conveyed by each element, allowing different genes to evolve distinct, often complex patterns of transcriptional regulation.

The term promoter will be used here to refer to a group of transcriptional control modules that are clustered around the initiation site for RNA polymerase II. Much of the thinking about how promoters are organized have shown that promoters are composed of discrete functional modules, each consisting of approximately 7-20 bp of DNA, and containing one or more recognition sites for transcriptional activator proteins. At least one module in each promoter functions to position the start site for RNA synthesis. The best known example of this is the TATA box, but in some promoters lacking a TATA box a discrete element overlying the start site itself helps to fix the place of initiation.

Additional promoter elements regulate the frequency of transcriptional initiation. Typically, these are located in the region 30-110 bp upstream of the start site, although a number of promoters have been shown to contain functional elements downstream of the start site as well. The spacing between elements is flexible, so that promoter function is preserved when elements are inverted or moved relative to one another. Depending on the promoter, it appears that individual elements can function either cooperatively or independently to activate transcription.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Aside from this operational distinction, enhancers and promoters are very similar entities. They have the same general function of activating transcription in the cell. They are often overlapping and contiguous, often seeming to have a very similar modular organization. Taken together, these considerations suggest that enhancers and promoters are homologous entities and that the transcriptional activator proteins bound to these sequences may interact with the cellular transcriptional machinery in fundamentally the same way.

In one preferred embodiment of the present invention there is provided a polynucleotide comprising

i) a first nucleotide sequence encoding at least one regulator of morphology capable of regulating the morphology of a dimorphic fungal cell, and operably linked thereto

ii) a second nucleotide sequence comprising an expression signal capable of directing the expression of the first nucleotide sequence in a dimorphic fungal cell,

wherein the first and second nucleotide sequences are not natively associated, and

wherein the second nucleotide sequence comprises an expression signal comprising at least one element of a promoter region capable of being regulated, during growth of a dimorphic fungal cell, by any one or more factors including

a) the composition of the growth medium, including at least one of carbon source, nitrogen source including amino acids or precursors thereof, oxygen content, ionic strength, including NaCl content, pH, low molecular weight compounds, cAMP, and the presence or absence of a cell constituent, or a precursor thereof,

b) the temperature of the growth medium, including any change thereof, including an upshift eliciting the expression of one or more heat shock genes,

c) the growth phase of the dimorphic fungal cell, and

d) the growth rate of the dimorphic fungal cell.

The regulation is preferably an induction or a derepression or a repression, including an induction, a derepression or a repression of the expression of the first nucleotide sequence being operably linked to the expression signal, as compared to a predetermined expression level, by at least a factor of 1.02, such as at least a factor 1.05, for example at least a factor 1.10, such as at least a factor 1.15, for example at least a factor 1.20, such as at least a factor 1.25, for example at least a factor 1.30, such as at least a factor 1.35, for example at least a factor 1.40, such as at least a factor 1.45, for example at least a factor 1.50, such as at least a factor 1.75, for example at least a factor 2.0, such as at least a factor 2,5, for example at least a factor 5.0, such as at least a factor 7.5, for example at least a factor 10, such as at least a factor 15, for example at least a factor 20, such as at least a factor 30, for example at least a factor 40, such as at least a factor 50, for example at least a factor 75, such as at least a factor 100, for example at least a factor 150, such as at least a factor 200, for example at least a factor 500, such as at least a factor 1000, for example at least a factor 2000, such as at least a factor 4000, for example at least a factor 8000, such as at least a factor 10000, for example at least a factor 15000, such as at least a factor 25000, for example at least a factor 50000, such as at least a factor 75000, for example at least a factor 100000.

Predetermined expression level as used herein signifies any expression level detectable by means of any assay including any determination or analysis of mRNA and/or polypeptide production prior to any change in any one or more of the above-mentioned factors.

Northern blots are used to measure e.g. induction of expression using a Cyclone Storage Phosphor System (Packard) and the OptiQuant image analysis software. A linear dynamic range of 5 orders of magnitude with only a 5% standard deviation is possible with the above apparatus. Dilution series of the RNA preparations (typically 20, 10 and 5 μg total RNA per sample) can be used to estimate induction levels.

Such a change in e.g. mRNA following e.g. induced gene expression may either result from an addition to the growth medium of the factor, said addition resulting in an increased or essentially constant concentration of said factor in the growth medium, or a removal from the growth medium of the factor, said removal resulting in a decreased concentration, or an essentially constant concentration, of said factor in the growth medium.

The addition may be an addition from an external source, or it may be an addition to the growth medium of a factor produced or consumed by the microbial cell in question, including a fungal cell, including a dimorphic fungal cell.

The removal from the growth medium of factor may be a selective removal, e.g. a filtration or precipitation of factor, resulting in a decreased concentration, or an essentially constant concentration, of said factor being present in the growth medium. Alternatively, the removal may be generated by consumption of the factor by the microbial cell in question, including a fungal cell, including a dimorphic fungal cell.

Accordingly, it is possible to determine—at a first predetermined timepoint during the cultivation of the microbial cell, including a fungal cell, including a dimorphic fungal cell—the predetermined level of expression of the first nucleotide sequence being operably linked to the expression signal, and to determine for that same timepoint, the composition of the growth medium, including at least one of carbon source, nitrogen source including amino acids or precursors thereof, oxygen content, ionic strength, including NaCl content, pH, low molecular weight compounds, cAMP, and the presence or absence of a cell constituent, or a precursor thereof.

Concomitant determinations at a later and second predetermined timepoint of i) the expression of the first nucleotide sequence, and ii) the relative change of the growth composition as compared to the growth composition at the first predetermined timepoint allows the skilled person to evaluate the effect of a) the change of the growth composition on b) the expression of the first nucleotide sequence operably linked to the expression signal.

This evaluation allows the skilled person to conclude whether the expression of the first nucleotide sequence encoding the at least one regulator of morphology is induced or repressed by the changed growth conditions. The skilled person will in one embodiment change only one factor at any one time and maintain the remaining factors at an essentially unchanged status or level as far as this is possible. In other embodiments, more than one factor is changed simultaneously, or sequentially in any order.

The change in the composition of the growth medium may lead to either an increased or a decreased expression of the first nucleotide sequence encoding the at least one regulator of morphology, said increased or decreased expression resulting—directly or indirectly—in an increased or a decreased production of regulator of morphology, respectively, said increased or decreased production of regulator results in an improved filamentation of a fungal cell, including a dimorphic fungal cell, or in a dimorphic shift of a dimorphic fungal cell. The dimorphic shift of the dimorphic fungal cell preferably also results in an improved filamentation.

It is understood that the increased or a decreased expression of the first nucleotide sequence results from the expression signal comprised in the second nucleotide sequence being regulatable by the change in the composition of the growth medium.

When the second nucleotide sequence comprising an expression signal is regulatable by at least one factor of a growth medium composition:

the carbon source is preferably e.g., starch, cellulose, pectin, oligosaccharides, glucose, galactose, glycerol, ethanol, and any change in the composition or amount of the carbon source regulate in one embodiment the expression signal comprised by the second nucleotide sequence,

the nitrogen source is preferably proteins, peptides (like casaminoacids), amino acids, including any composition of naturally occurring amino acids, and precursors and/or derivatives thereof, like citrulline, ornithine, and the like, as well as inorganic salts (like ammonium sulfate, acetamide, nitrates or nitrites), and any change in the composition or amount of the nitrogen source regulate in one embodiment the expression signal comprised by the second nucleotide sequence,

the oxygen content is preferably one characterised as sufficient to result in aerobic growth, or an oxygen content sufficiently low to qualify growth conditions characterised as anaerobic growth. Anaerobic is per definition an atmosphere with no oxygen. Mucor circinelloides adopts a filamentious morphology when levels of oxygen are above 1.2 micromolar. At lower levels Mucor circinelloides grows with a unicellular, essentially spherical morphology provided that a fermentable hexose and organic nitrogen are present. However, Mucor circinelloides can grow as filaments in lower concentrations of oxygen (also complete anaerobic atmosphere) in case of physiological stress. Also the opposite situation is observed: aerobic yeast growth may occur under specific growth conditions (e.g., high glucose concentration after the shift from anaerobic growth to aerobic growth). In one embodiment, changes in oxygen levels regulate the expression signal comprised by the second nucleotide sequence,

the ionic strength, including NaCl content, is preferably that of a standard growth medium, and changes therein regulate the expression signal comprised by the second nucleotide sequence in one embodiment of the present invention,

the pH is preferably within the range of physiological pH values, and changes therein regulate the expression signal comprised by the second nucleotide sequence in one embodiment,

low molecular weight compounds are preferably salts (sulfate, phosphate, nitrate), and/or metals (e.g., copper), and changes therein regulate the expression signal comprised by the second nucleotide sequence in one embodiment,

cAMP levels are preferably those sufficient for inducing an improved filamentation and/or a dimorphic shift. Mucor species having a unicellular, essentially spherical morphology may have an intracellular cAMP level of about 2-10 mM intracellular cAMP, while filamentous growing Mucor species generally have lower levels of cAMP. When added exogenously, about 40 mM dbcAMP preferably induces a dimorphic shift from aerobic, Mucor filamentous growth to aerobic, Mucor unicellular, essentially spherical morphology. Lower concentrations such as e.g. 10 mM dbcAMP may also work for some species. In one embodiment, changes in cAMP levels regulate the expression signal comprised by the second nucleotide sequence,

presence or absence of a cell constituent, or a precursor thereof, is preferably a cofactor, a vitamin, or a lipid, and the like, and in one embodiment, changes in cell constituent levels regulate the expression signal comprised by the second nucleotide sequence.

It is preferred that the second polynucleotide comprises at least one element of a promoter region comprised by the expression signal, preferably a promoter element regulated, during growth of the dimorphic fungal cell, by the carbon source of the growth medium and/or the oxygen content and the carbon source of the growth medium. It is preferred that the at least one element of the promoter region is induced by the presence in the growth medium, or the addition to the growth medium, of a carbon source. The carbon source preferably comprises a hexose, preferably glucose and/or galactose.

Promoter Elements of Second Nucleotide Sequences

gpd1P

In one embodiment, the present invention relates to an expression signal comprising a promoter element isolated from a Mucor circinelloides glyceraldehyde-3-phosphate dehydrogenase promoter, preferably gpd1P, including, but not limited to, a promoter element comprising nucleotides 1 to 741 of SEQ ID NO:9, or a fragment thereof capable of directing gene expression in a fungal host cell.

Accordingly, in one embodiment of the present invention at least one element of the promoter region comprised by the expression signal is selected from the group consisting of

i) a polynucleotide comprising nucleotides 1 to 741 of SEQ ID NO:9,

ii) a polynucleotide comprising or essentially consisting of the promoter region of gpd1 of Mucor circinelloides, as deposited with DSMZ under accession number DSM 14066; and

iii) a polynucleotide comprising at least one fragment of SEQ ID NO:9, wherein said fragment

a) is capable of directing gene expression in a dimorphic fungal cell; and/or

b) is regulatable, during growth of the dimorphic fungal cell, by at least one factor capable of regulating gene expression directed by nucleotides 1 to 741 of SEQ ID NO:9; and/or

c) is capable of being regulated, during growth of the dimorphic fungal cell, by one or more of

the composition of the growth medium, including at least one of carbon source, nitrogen source including amino acids or precursors thereof, oxygen content, ionic strength, including NaCl content, pH, low molecular weight compounds, cAMP, and the presence or absence of a cell constituent, or a precursor thereof,

the temperature of the growth medium, including any change thereof, including an upshift eliciting the expression of one or more heat shock genes,

the growth phase of the dimorphic fungal cell, and

the growth rate of the dimorphic fungal cell, and

iv) a polynucleotide, the complementary strand of which hybridizes, under stringent conditions, with a polynucleotide as defined in any of (i), (ii) and (iii), wherein said polynucleotide

a) is capable of directing gene expression in a dimorphic fungal cell; and/or

b) is regulatable, during growth of the dimorphic fungal cell, by at least one factor capable of regulating gene expression directed by nucleotides 1 to 741 of SEQ ID NO:9,

and the complementary strand of such a polynucleotide.

The expression signal comprising a promoter element isolated from a Mucor circinelloides glyceraldehyde-3-phosphate dehydrogenase promoter, preferably gpd1P, is in one embodiment also capable of directing gene expression in other fungal cells including fungal cells that are not dimorphic fungal cells. At least one promoter element of gpd1P is induced by the carbon source of the growth medium, said induction resulting in an increased expression of the at least one regulator of morphology being encoded by a nucleotide sequence operably linked to said promoter element.

In one embodiment, the at least one element of the promoter region comprised by the expression signal comprises nucleotides 1 to 741 of SEQ ID NO:9, or a fragment thereof, wherein said fragment is capable of directing gene expression in a fungal cell, including a dimorphic fungal cell, and is regulatable, during growth of the fungal cell, including a dimorphic fungal cell, by at least one factor capable of regulating gene expression directed by nucleotides 1 to 741 of SEQ ID NO:9. The factor is preferably selected from the group consisting of

a) the composition of the growth medium, including at least one of carbon source, nitrogen source including amino acids or precursors thereof, oxygen content, ionic strength, including NaCl content, pH, low molecular weight compounds, cAMP, and the presence or absence of a cell constituent, or a precursor thereof,

b) the temperature of the growth medium, including any change thereof, including an upshift eliciting the expression of one or more heat shock genes,

c) the growth phase of the dimorphic fungal cell, and

d) the growth rate of the dimorphic fungal cell.

Also, the at least one element of the promoter region comprised by the expression signal preferably comprises nucleotides 1 to 741 of SEQ ID NO: 9, or the promoter region of gpd1 of M. circinelloides, as deposited with DSMZ under accession number DSM 14066.

Fragments of SEQ ID NO:9 are preferably less than 600 nucleotides, such as less than 500 nucleotides, for example less than 400 nucleotides, such as less than 350 nucleotides, for example less than 300 nucleotides, for example less than 275 nucleotides, such as less than 250 nucleotides, for example less than 200 nucleotides, for example less than 150 nucleotides, such as less than 100 nucleotides.

prnCP

In another embodiment, the present invention relates to an expression signal comprising a promoter element isolated from the promoter region of Mucor circinelloides prnC, including, but not limited to, a promoter element comprising nucleotides 1 to 755 of SEQ ID NO:10, or a fragment thereof capable of directing gene expression in a fungal host cell.

Accordingly, in one embodiment of the present invention, the at least one element of the promoter region comprised by the expression signal is preferably selected from the group consisting of

i) a polynucleotide comprising nucleotides 1 to 755 of SEQ ID NO:10,

ii) a polynucleotide comprising or essentially consisting of the promoter region of prnC of Mucor circinelloides, as deposited with DSMZ under accession number DSM 14067; and

iii) a polynucleotide comprising at least one fragment of SEQ ID NO:10, wherein said fragment

a) is capable of directing gene expression in a dimorphic fungal cell; and/or

b) is regulatable, during growth of the dimorphic fungal cell, by at least one factor capable of regulating gene expression directed by nucleotides 1 to 755 of SEQ ID NO:10; and/or

c) is capable of being regulated, during growth of the dimorphic fungal cell, by one or more of

the composition of the growth medium, including at least one of carbon source, nitrogen source including amino acids or precursors thereof, oxygen content, ionic strength, including NaCl content, pH, low molecular weight compounds, cAMP, and the presence or absence of a cell constituent, or a precursor thereof

the temperature of the growth medium, including any change thereof, including an upshift eliciting the expression of one or more heat shock genes,

the growth phase of the dimorphic fungal cell, and

the growth rate of the dimorphic fungal cell, and

iv) a polynucleotide, the complementary strand of which hybridizes, under stringent conditions, with a polynucleotide as defined in any of (i), (ii) and (iii), wherein said polynucleotide

a) is capable of directing gene expression in a dimorphic fungal cell; and/or

b) is regulatable, during growth of the dimorphic fungal cell, by at least one factor capable of regulating gene expression directed by nucleotides 1 to 755 of SEQ ID NO:10,

and the complementary strand of such a polynucleotide.

The expression signal comprising a promoter element isolated from Mucor circinelloides prnC is in one embodiment also capable of directing gene expression in other fungal cells including fungal cells that are not dimorphic fungal cells. At least one promoter element of prnC is induced by aerobicity during filamentous growth of a fungal cell, including a dimorphic fungal cell, said induction resulting in an increased expression of the at least one regulator of morphology being encoded by a nucleotide sequence operably linked to said promoter element.

The at least one element of the promoter region comprised by the expression signal preferably comprises nucleotides 1 to 755 of SEQ ID NO:10, or a fragment thereof, wherein said fragment is capable of directing gene expression in a fungal cell, including a dimorphic fungal cell, and is regulatable, during growth of the dimorphic fungal cell, by at least one factor capable of regulating gene expression directed by nucleotides 1 to 755 of SEQ ID NO:10. The factor is preferably selected from the group consisting of

a) the composition of the growth medium, including at least one of carbon source, nitrogen source including amino acids or precursors thereof, oxygen content, ionic strength, including NaCl content, pH, low molecular weight compounds, cAMP, and the presence or absence of a cell constituent, or a precursor thereof,

b) the temperature of the growth medium, including any change thereof, including an upshift eliciting the expression of one or more heat shock genes,

c) the growth phase of the dimorphic fungal cell, and

d) the growth rate of the dimorphic fungal cell.

In another preferred embodiment, the at least one element of the promoter region comprised by the expression signal comprises nucleotides 1 to 755 of SEQ ID NO: 10, or the promoter region of prnC of M. circinelloides, as deposited with DSMZ under accession number DSM 14067.

Fragments of SEQ ID NO:10 are preferably less than 600 nucleotides, such as less than 500 nucleotides, for example less than 400 nucleotides, such as less than 350 nucleotides, for example less than 300 nucleotides, for example less than 275 nucleotides, such as less than 250 nucleotides, for example less than 200 nucleotides, for example less than 150 nucleotides, such as less than 100 nucleotides.

tubAP

There is also provided an embodiment, wherein at least one element of the promoter region comprised by the expression signal is selected from the group consisting of

i) a polynucleotide comprising nucleotides 1 to 927 of SEQ ID NO:13,

ii) a polynucleotide comprising or essentially consisting of the promoter region of tubA of Mucor circinelloides, as deposited with DSMZ under accession number DSM 14841; and

iii) a polynucleotide comprising at least one fragment of SEQ ID NO:13, wherein said fragment

a) is capable of directing gene expression in a dimorphic fungal cell; and/or

b) is regulatable, during growth of the dimorphic fungal cell, by at least one factor capable of regulating gene expression directed by SEQ ID NO:13; and

c) is capable of being regulated, during growth of the dimorphic fungal cell, by one or more of

the composition of the growth medium, including at least one of carbon source, nitrogen source including amino acids or precursors thereof, oxygen content, ionic strength, including NaCl content, pH, low molecular weight compounds, cAMP, and the presence or absence of a cell constituent, or a precursor thereof

the temperature of the growth medium, including any change thereof, including an upshift eliciting the expression of one or more heat shock genes,

the growth phase of the dimorphic fungal cell, and

the growth rate of the dimorphic fungal cell, and

iv) a polynucleotide, the complementary strand of which hybridizes, under stringent conditions, with a polynucleotide as defined in any of (i), (ii) and (iii), wherein said polynucleotide

a) is capable of directing gene expression in a dimorphic fungal cell; and/or

b) is regulatable, during growth of the dimorphic fungal cell, by at least one factor capable of regulating gene expression directed by SEQ ID NO:13,

and the complementary strand of such a polynucleotide.

Accordingly, there is provided in one embodiment at least one further nucleotide sequence comprising nucleotides 1 to 927 of SEQ ID NO:13.

In another embodiment there is provided a polynucleotide comprising or essentially consisting of the promoter region of tubA of Mucor circinelloides, as deposited with DSMZ under accession number DSM 14841.

In a still further embodiment, there is provided a polynucleotide comprising at least one fragment of SEQ ID NO:13, wherein said fragment

a) is capable of directing gene expression in a dimorphic fungal cell; and/or

b) is regulatable, during growth of the dimorphic fungal cell, by at least one factor capable of regulating gene expression directed by SEQ ID NO:13.

In a still further embodiment, there is provided a polynucleotide, the complementary strand of which hybridizes, under stringent conditions, with a polynucleotide as defined in any of (i), (ii) and (iii), wherein said polynucleotide

a) is capable of directing gene expression in a dimorphic fungal cell; and/or

b) is regulatable, during growth of the dimorphic fungal cell, by at least one factor capable of regulating gene expression directed by SEQ ID NO:13.

Fragments of SEQ ID NO:13 are preferably less than 500 nucleotides, such as less than 400 nucleotides, for example less than 350 nucleotides, for example less than 300 nucleotides, such as less than 250 nucleotides, for example less than 200 nucleotides, for example less than 150 nucleotides, such as less than 100 nucleotides.

gal1P

There is also provided an embodiment, wherein the at least one further nucleotide sequence comprising the further expression signal is selected from the group consisting of

i) a polynucleotide comprising nucleotides 1 to 419 of SEQ ID NO:14;

ii) a polynucleotide comprising or essentially consisting of the promoter region of gal1 of Mucor circinelloides, as deposited with EMBL under accession number AJ438267; and

iii) a polynucleotide comprising at least one fragment of SEQ ID NO:14, wherein said fragment

a) is capable of directing gene expression in a dimorphic fungal cell; and/or

b) is regulatable, during growth of the dimorphic fungal cell, by at least one factor capable of regulating gene expression directed by SEQ ID NQ:14; and

c) is capable of being regulated, during growth of the dimorphic fungal cell, by one or more of

the composition of the growth medium, including at least one of carbon source, nitrogen source including amino acids or precursors thereof, oxygen content, ionic strength, including NaCl content, pH, low molecular weight compounds, cAMP, and the presence or absence of a cell constituent, or a precursor thereof

the temperature of the growth medium, including any change thereof, including an upshift eliciting the expression of one or more heat shock genes,

the growth phase of the dimorphic fungal cell, and

the growth rate of the dimorphic fungal cell, and

iv) a polynucleotide, the complementary strand of which hybridizes, under stringent conditions, with a polynucleotide as defined in any of (i), (ii) and (iii), wherein said polynucleotide

a) is capable of directing gene expression in a dimorphic fungal cell; and/or

b) is regulatable, during growth of the dimorphic fungal cell, by at least one factor capable of regulating gene expression directed by SEQ ID NO:14,

and the complementary strand of such a polynucleotide.

Accordingly, there is provided in one embodiment at least one further nucleotide sequence comprising nucleotides 1 to 419 of SEQ ID NO:14.

In another embodiment there is provided a polynucleotide comprising or essentially consisting of the promoter region of gal1 of Mucor circinelloides, as deposited with EMBL under accession number AJ438267.

In a still further embodiment, there is provided a polynucleotide comprising at least one fragment of SEQ ID NO:14, wherein said fragment

a) is capable of directing gene expression in a dimorphic fungal cell; and/or

b) is regulatable, during growth of the dimorphic fungal cell, by at least one factor capable of regulating gene expression directed by SEQ ID NO:14.

In a still further embodiment, there is provided a polynucleotide, the complementary strand of which hybridizes, under stringent conditions, with a polynucleotide as defined in any of (i), (ii) and (iii), wherein said polynucleotide

a) is capable of directing gene expression in a dimorphic fungal cell; and/or

b) is regulatable, during growth of the dimorphic fungal cell, by at least one factor capable of regulating gene expression directed by SEQ ID NO:14.

Fragments of SEQ ID NO:14 are preferably less than 400 nucleotides, such as less than 350 nucleotides, for example less than 300 nucleotides, for example less than 275 nucleotides, such as less than 250 nucleotides, for example less than 200 nucleotides, for example less than 150 nucleotides, such as less than 100 nucleotides.

In a further embodiment, there is provided a polynucleotide as listed herein above operably linked to a further polynucleotide selected from the group of polynucleotides consisting of a 3′ untranslated region, or a fragment thereof, and/or a 5′ upstream region, or a fragment thereof.

Regulators Produced from Expression of First Nucleotide Sequences

The at least one regulator of morphology is in one embodiment a polypeptide capable of regulating gene transcription in a dimorphic fungal cell by forming an interaction with a recognition motif of a promoter region having an affinity for the at least one regulator of morphology. In another embodiment the at least one regulator of morphology comprises a kinase activity, or comprises a regulator of a kinase activity, or is regulated by a kinase activity.

In one embodiment, the expression of the first nucleotide sequence directed by the second nucleotide sequence results in the production of the at least one regulator of morphology, and said production results in the dimorphic fungal cell adopting a unicellular, essentially spherical morphology. In the absence of production of the at least one regulator of morphology, the cell adopts a filamentous morphology and grows in the form of a mycelium having a filamentous structure and comprising multinucleated or uninucleated cells. When the cell is a zygomycete, the individual cells are multinucleated. When the cell is e.g. Candida or Arxula, the individual cells are uninucleated.

Accordingly, in an embodiment wherein the production of the at least one regulator of morphology results in the dimorphic fungal cell adopting a unicellular, essentially spherical morphology, and wherein the absence of production of the at least one regulator of morphology results in the dimorphic fungal cell adopting a filamentous morphology, the present invention relates to a regulatably reduced production of such a regulator of morphology.

In the above-mentioned embodiment wherein the invention relates to a repressor capable of repression of a dimorphic shift, or capable of repression of filamentation, or capable of repression of an improved filamentation in a dimorphic fungal cell, the lifting of the repression (de-repression), by means of reducing or eliminating the expression of the first nucleotide sequence encoding the repressor, preferably results in a dimorphic shift and/or an improved filamentation of the dimorphic fungal cell.

In another embodiment, the regulator of morphology can be an activator of unicellular, essentially spherical morphology. In such a case, when expression of the activator ceases or decreases, the dimorphic shift will occur.

For both of the above embodiments, the dimorphic shift may be further aided by the expression of an activator of filamentous growth, or by expression of a repressor of unicellular, essentially spherical morphology.

Accordingly, in order to further promote a dimorphic shift, or in order to further improve the filamentation of the dimorphic fungal cell, the cell may comprise a further regulator of morphology encoded by a nucleotide sequence not natively associated with said further regulator of morphology, wherein the production of said further regulator of morphology is positively correlated with a dimorphic shift and/or an improved filamentation of the dimorphic fungal cell. It is preferred in one embodiment that the further regulator of morphology is an activator, the production of which results in a dimorphic shift and/or an improved filamentation of the dimorphic fungal cell.

An activator may be present in the dimorphic cell irrespective of whether the above-mentioned repressor is present or not. It will be understood that both the repressor and the activator are encoded by a nucleotide sequence operably linked to a regulatable promoter not natively associated therewith, including a second nucleotide sequence.

Accordingly, in a further embodiment, the invention relates to a regulator of morphology in the form of an activator, the expression of which results in a dimorphic shift, or results in filamentation, or results in an improved filamentation in a dimorphic fungal cell, said activator being encoded by a nucleotide sequence operably linked to a regulatable promoter not natively associated therewith, including a second nucleotide sequence.

In one embodiment, the activator is not expressed, or expressed at a reduced level, when the microbial cell, including a fungal cell, including a dimorphic fungal cell, adopts a unicellular, essentially spherical morphology. Following induction, increased expression or otherwise, the production of the activator results in the cell adopting a filamentous morphology and growing in the form of a mycelium having a filamentous structure and comprising multinucleated or uninucleated cells. When the cell is a zygomycete, the individual cells are multinucleated. When the cell is e.g. Geotrichum, Candida or Arxula, the individual cells are uninucleated.

In the above-mentioned embodiment wherein the invention relates to activator, the induction of the activation, by means of increasing the expression of the first nucleotide sequence encoding the activator, preferably results in a dimorphic shift and/or an improved filamentation of the dimorphic fungal cell.

In order to further promote a dimorphic shift, or in order to further improve the filamentation of the dimorphic fungal cell, the cell may comprise a further activator encoded by a nucleotide sequence not natively associated with said further activator, wherein the production of said further activator results in a dimorphic shift and/or an improved filamentation of the dimorphic fungal cell.

When the at least one regulator of morphology directs a dimorphic shift of a dimorphic fungal cell, the shift is in one embodiment a shift from a first morphological condition of the dimorphic fungal cell, wherein the fungal cell has an unicellular, essentially spherical morphology, to a second morphological condition of the dimorphic fungal cell, wherein the fungal cell is characterised by a mycelium capable of filamentous growth.

A shift from a first morphological condition wherein the fungal cell has an unicellular, essentially spherical morphology, to a second morphological condition wherein the fungal cell is characterised by a mycelium capable of filamentous growth, can occur as described in the below table.

As illustrated in the table, controlled dimorphic shifts can be obtained in various ways. In the first example, initial growth in medium containing high glucose will lead to high level expression of pkaC and repression of expression of pkaR and consequently to unicellular, essentially spherical growth. As glucose is depleted, expression of pkaC decreases leading to derepression of pkaR expression. Further increase of pkaR expression can be aided with the addition of galactose to the medium. The increase in pkaR expression leads to filamentation. In the second example, a high level of expression of pkaC is maintained throughout. In medium with an initial high level of glucose, expression of pkaR is repressed, leading to unicellular, essentially spherical growth. Depletion of glucose and/or addition of galactose results in an increase in pkaR expression leading to filamentation. The additional examples depicted are modifications to the above strategies.

| | Induction of | Induction of | |
|---|---|---|---|
| | activator of | repressor of | |
| | unicellular, | unicellular, | |
| | essentially | essentially | Expression of |
| | spherical | spherical | regulator(s) during |
| Construction | morphology | morphology | growth |
| gpd1P- | Glucose | Glucose | pkaC decreases with |
| pkaC/gal1P- | concentration | consumption | glucose consumption |
| pkaR | | and | or is maintained |
| | | galactose | (addition of galac- |
| | | addition | tose); pkaR increases |
| | | | with glucose con- |
| | | | sumption and/or |
| | | | addition of galactose |
| tubAP-pkaC/ | Maintained high | Galactose | pkaC high level; pkaR |
| gal1P-pkaR | expression | addition | increases with |
| | | | galactose addition |
| tubAP-pkaC/ | Maintained high | Oxidative | pkaC high level; pkaR |
| prnCP-pkaR | expression | metabolism | increases |
| gpd1P- | Glucose concentra- | Oxidative | pkaC decreases with |
| pkaC/prnCP- | tion | metabolism | glucose consumption; |
| pkaR | | | pkaR increases |
| gal1P-pkaC/ | Galactose concen- | Oxidative | pkaC decreases with |
| prnCP-pkaR | tration | metabolism | galactose consump- |
| | | | tion; pkaR increases |

Accordingly, in preferred embodiments there is provided:

Fungal cell or dimorphic fungal cell comprising i) a first nucleotide sequence comprising the coding sequence of SEQ ID NO:11, or a fragment thereof encoding a regulator of morphology of said fungal cell or said dimorphic fungal cell, said first sequence being operably linked to a second nucleotide sequence comprising SEQ ID NO:9, or a fragment thereof capable of directing gene expression in said fungal cell or said dimorphic fungal cell, said cell further comprising ii) a first nucleotide sequence comprising the coding sequence of SEQ ID NO:1, or a fragment thereof encoding a regulator of morphology of said fungal cell or said dimorphic fungal cell, said first sequence being operably linked to a second nucleotide sequence comprising SEQ ID NO:14, or a fragment thereof capable of directing gene expression in said fungal cell or said dimorphic fungal cell.

Fungal cell or dimorphic fungal cell comprising i) a first nucleotide sequence comprising the coding sequence of SEQ ID NO:11, or a fragment thereof encoding a regulator of morphology of said fungal cell or said dimorphic fungal cell, said first sequence being operably linked to a second nucleotide sequence comprising SEQ ID NO:13, or a fragment thereof capable of directing gene expression in said fungal cell or said dimorphic fungal cell, said cell further comprising ii) a first nucleotide sequence comprising the coding sequence of SEQ ID NO:1, or a fragment thereof encoding a regulator of morphology of said fungal cell or said dimorphic fungal cell, said first sequence being operably linked to a second nucleotide sequence comprising SEQ ID NO:14, or a fragment thereof capable of directing gene expression in said fungal cell or said dimorphic fungal cell.

Fungal cell or dimorphic fungal cell comprising i) a first nucleotide sequence comprising the coding sequence of SEQ ID NO:11, or a fragment thereof encoding a regulator of morphology of said fungal cell or said dimorphic fungal cell, said first sequence being operably linked to a second nucleotide sequence comprising SEQ ID NO:13, or a fragment thereof capable of directing gene expression in said fungal cell or said dimorphic fungal cell, said cell further comprising ii) a first nucleotide sequence comprising the coding sequence of SEQ ID NO:1, or a fragment thereof encoding a regulator of morphology of said fungal cell or said dimorphic fungal cell, said first sequence being operably linked to a second nucleotide sequence comprising SEQ ID NO:10, or a fragment thereof capable of directing gene expression in said fungal cell or said dimorphic fungal cell.

Fungal cell or dimorphic fungal cell comprising i) a first nucleotide sequence comprising the coding sequence of SEQ ID NO:11, or a fragment thereof encoding a regulator of morphology of said fungal cell or said dimorphic fungal cell, said first sequence being operably linked to a second nucleotide sequence comprising SEQ ID NO:9, or a fragment thereof capable of directing gene expression in said fungal cell or said dimorphic fungal cell, said cell further comprising ii) a first nucleotide sequence comprising the coding sequence of SEQ ID NO:1, or a fragment thereof encoding a regulator of morphology of said fungal cell or said dimorphic fungal cell, said first sequence being operably linked to a second nucleotide sequence comprising SEQ ID NO:10, or a fragment thereof capable of directing gene expression in said fungal cell or said dimorphic fungal cell.

Fungal cell or dimorphic fungal cell comprising i) a first nucleotide sequence comprising the coding sequence of SEQ ID NO:11, or a fragment thereof encoding a regulator of morphology of said fungal cell or said dimorphic fungal cell, said first sequence being operably linked to a second nucleotide sequence comprising SEQ ID NO:14, or a fragment thereof capable of directing gene expression in said fungal cell or said dimorphic fungal cell, said cell further comprising ii) a first nucleotide sequence comprising the coding sequence of SEQ ID NO:1, or a fragment thereof encoding a regulator of morphology of said fungal cell or said dimorphic fungal cell, said first sequence being operably linked to a second nucleotide sequence comprising SEQ ID NO:10, or a fragment thereof capable of directing gene expression in said fungal cell or said dimorphic fungal cell.

In another embodiment, the dimorphic shift of the dimorphic fungal cell is a shift from a second morphological condition of the dimorphic fungal cell, wherein the fungal cell is characterised by a mycelium capable of filamentous growth, to a first morphological condition of the dimorphic fungal cell, wherein the fungal cell has an unicellular, essentially spherical morphology.

In a further embodiment, the first morphological condition, wherein the fungal cell has an unicellular, essentially spherical morphology, is further characterised by an essentially isodiametrical or spherical shape of the fungal cells, or by an essentially non-polarised growth of the cells. The second morphological condition, characterised by filamentous growth, is further characterised by an essentially elongated, hyphal cell shape resulting from a polarised growth of a fungal cell characterised by the first morphological condition.

Although a dimorphic shift can only be observed for a dimorphic fungal cell, an improved filamentation can result from the expression of a regulator of morphology in any microbial cell capable of displaying a filamentous morphology, including any filamentous fungal cell, including any zygomycete.

Accordingly, there is provided in one embodiment, a nucleotide sequence encoding a regulator of morphology, wherein an increased or decreased expression of said regulator results in an improved filamentation of a fungal cell without inducing concomitantly therewith a dimorphic shift when the cell is a dimorphic fungal cell, including a dimorphic fungal cell, the morphology of which is regulatable by the at least one regulator of morphology and wherein the cell is capable of growing as a multinucleated cell having a unicellular, essentially spherical morphology and/or capable of growing as a mycelium having a filamentous structure and comprising multinucleated cells, including dimorphic fungal cells comprising multinucleated and multipolar cells, and dimorphic fungal cells that are multinucleated as well as bipolar or monopolar. In one embodiment, the dimorphic fungal cell belongs to the class of Zygomycetes, including the order of Mucorales, including from the order of Mucorales a genus preferably selected from the group of genera consisting of Mucor, Thermomucor, Rhizomucor, Mycotypha, Rhizopus, and Cokeromyces, including Cokeromyces recurvatus. Preferred Mucor species according to this embodiment of the invention are those mentioned herein above, including M. circinelloides, M. hiemalis, M. rouxii, M. genevensis, M. bacilliformis, and M. subtillissimus. M. circinelloides is particularly preferred.

In a still further embodiment, there is provided a nucleotide sequence encoding a regulator of morphology, wherein an increased or decreased expression of said regulator results in an improved filamentation of a fungal cell that is not a dimorphic cell. Non-limiting examples of such cells are listed herein above.

In one preferred embodiment, the expression of said first polynucleotide results in the production of the at least one regulator in an increased or decreased amount, as compared to the amount of regulator produced, when the first polynucleotide encoding the at least one regulator is operably linked to the native promoter region under substantially identical growth conditions, and wherein the expression of said first polynucleotide and the production of the at least one regulator in an increased or a decreased amount results in an improved filamentation, or in a dimorphic shift of the dimorphic fungal cell. Substantially identical growth conditions are conditions characterised by deviations of growth composition factors within a 5 percent range, including deviations within a range characterised by standard deviations of measurement.

Preferably, an increased amount of the at least one regulator of morphology is produced from the expression of the first nucleotide sequence encoding said regulator. However, the invention also relates to an embodiment, wherein a decreased amount of the at least one regulator of morphology is produced from the expression of the first nucleotide sequence encoding said regulator.

The amount of regulator produced is preferably increased or decreased at least by a factor of 1.02, such as at least by a factor 1.05, for example at least by a factor 1.10, such as at least a factor 1.15, for example at least by a factor 1.20, such as at least a factor 1.25, for example at least by a factor 1.30, such as at least a factor 1.35, for example at least a factor 1.40, such as at least a factor 1.45, for example at least by a factor 1.50, such as at least by a factor 1.75, for example at least a factor 2.0, such as at least a factor 2,5, for example at least a factor 5.0, such as at least a factor 7.5, for example at least a factor 10, such as at least a factor 15, for example at least a factor 20, such as at least a factor 30, for example at least by a factor 40, such as at least a factor 50, for example at least by a factor 75, such as at least a factor 100, for example at least a factor 150, such as at least a factor 200, for example at least a factor 500, such as at least 1000, for example at least 5000, such as at least 10000, such as at least 20000, for example at least 40000, such as at least 60000, such as at least 80000, for example at least 100000.

Regulators of Signal Transduction Pathways Dependent on cAMP and MAP-kinase

In one embodiment, a regulator of morphology is related to a polypeptide encoded by a first polynucleotide comprising a first polynucleotide sequence forming part of a cAMP-dependent signal transduction pathway of a microbial cell, including a fungal cell, including a Mucor species, including M. circinelloides,

wherein the expression of said first polynucleotide results in the production of the at least one regulator in an increased or decreased amount, as compared to the amount of regulator produced, when the first polynucleotide encoding the at least one regulator is operably linked to the native promoter region under substantially identical growth conditions,

wherein the expression of said first polynucleotide and the production of the at least one regulator in an increased or a decreased amount results in an improved filamentation, or in a dimorphic shift of the dimorphic fungal cell.

In another embodiment, the at least one regulator of morphology is preferably a polypeptide encoded by a first polynucleotide comprising a first polynucleotide sequence forming part of a MAP kinase-dependent signal transduction pathway of a microbial cell, including a fungal cell, including a Mucor species, including M. circinelloides,

wherein the expression of said first polynucleotide results in the production of the at least one regulator in an increased or decreased amount, as compared to the amount of regulator produced, when the first polynucleotide encoding the at least one regulator is operably linked to the native promoter region under substantially identical growth conditions,

wherein the expression of said first polynucleotide and the production of the at least one regulator in an increased or a decreased amount results in an improved filamentation, or in a dimorphic shift of the dimorphic fungal cell.

Polypeptides

The present invention also pertains to polypeptides in the form of regulators of morphology encoded by the first nucleotide sequence as described herein above.

In one preferred embodiment, there is provided an isolated polypeptide comprising or essentially consisting of the amino acid sequence of SEQ ID NO:2, or a fragment thereof, or a polypeptide functionally equivalent to SEQ ID NO: 2, or a fragment thereof, wherein said fragment or functionally equivalent polypeptide

a) has Mucor circinelloides protein kinase A regulatory subunit acitivty and is a regulator of morphology of a dimorphic fungal cell; and/or

b) is recognised by an antibody, or a binding fragment thereof, which is capable of recognising a cAMP binding domain of Mucor circinelloides protein kinase A, wherein said cAMP binding domain is comprised by the polypeptide having the amino acid sequence as shown in SEQ ID NO:2; and/or

c) is competing with a polypeptide comprising or essentially consisting of the amino acid sequence as shown in SEQ ID NO:2 for binding to at least one predetermined binding partner, including cAMP and/or the catalytic subunit for protein kinase A.

In another preferred embodiment, there is provided an isolated polypeptide comprising or essentially consisting of the amino acid sequence of SEQ ID NO:12, or a fragment thereof, or a polypeptide functionally equivalent to SEQ ID NO:12, or a fragment thereof, wherein said fragment or functionally equivalent polypeptide

a) has Mucor circinelloides catalytic subunit of protein kinase A activity and is a regulator of morphology of a dimorphic fungal cell; and/or

b) is recognised by an antibody, or a binding fragment thereof, which is capable of recognising a protein kinase A binding domain of Mucor circinelloides PKAC, wherein said domain is comprised by the polypeptide having the amino acid sequence as shown in SEQ ID NO:12; and/or

c) is competing with a polypeptide comprising or essentially consisting of the amino acid sequence as shown in SEQ ID NO:12 for binding to at least one predetermined binding partner, including PKAR.

In yet another preferred embodiment there is provided an isolated polypeptide comprising or essentially consisting of the amino acid sequence of SEQ ID NO:4, or a fragment thereof, or a polypeptide functionally equivalent to SEQ ID NO: 4, or a fragment thereof, wherein said fragment or functionally equivalent polypeptide

a) has Mucor circinelloides STE20 acitivty and is a regulator of morphology of a dimorphic fungal cell; and/or

b) is recognised by an antibody, or a binding fragment thereof, which is capable of recognising the catalytic domain of Mucor circinelloides STE20, wherein said catalytic domain is comprised by the polypeptide having the amino acid sequence as shown in SEQ ID NO:4; and/or

c) is competing with a polypeptide comprising or essentially consisting of the amino acid sequence as shown in SEQ ID NO:4 for interaction with a substrate for phosphorylation, including triphosphate nucleotides, including ATP.

In yet another embodiment there is provided an isolated polypeptide comprising or essentially consisting of the amino acid sequence of SEQ ID NO:6, or a fragment thereof, or a polypeptide functionally equivalent to SEQ ID NO: 6, or a fragment thereof, wherein said fragment or functionally equivalent polypeptide

a) has Mucor circinelloides mitogen activated protein kinase 1 activity and is a regulator of morphology of a dimorphic fungal cell; and/or

b) is recognised by an antibody, or a binding fragment thereof, which is capable of recognising the catalytic domain of Mucor circinelloides mitogen activated protein kinase 1, wherein said catalytic domain is comprised by the polypeptide having the amino acid sequence as shown in SEQ ID NO:6; and/or

c) is competing with a polypeptide comprising or essentially consisting of the amino acid sequence as shown in SEQ ID NO:6 for interaction with a substrate for phosphorylation, including triphosphate nucleotides, including ATP.

In a still further embodiment, there is provided an isolated polypeptide comprising or essentially consisting of the amino acid sequence of SEQ ID NO:8, or a fragment thereof, or a polypeptide functionally equivalent to SEQ ID NO: 8, or a fragment thereof, wherein said fragment or functionally equivalent polypeptide

a) has Mucor circinelloides STE12 activity and is a regulator of morphology of a dimorphic fungal cell; and/or

b) is recognised by an antibody, or a binding fragment thereof, which is i) capable of recognising an N-terminal region of STE12 involved in polynucleotide binding and/or ii) capable of recognising an induction domain located in the central region of STE12 and/or iii) capable of recognising a C-terminal region of STE12 involved in transcriptional activation, wherein said recognised region or domain is comprised by the polypeptide having the amino acid sequence as shown in SEQ ID NO:8; and/or

c) is competing with a polypeptide having the amino acid sequence as shown in SEQ ID NO:8 for binding to a predetermined binding partner, including a polynucleotide having an affinity for said polypeptide.

Functional Equivalents and Variants of Polynucleotides Encoding a Regulator of Morphology and Polypeptides Comprising such a Regulator

As certain amino acids may be substituted for other amino acids in a polypeptide structure without appreciable loss of interactive binding capacity, and as it is the interactive capacity and nature of a polypeptide that defines the biological activity of the polypeptide, certain amino acid sequence substitutions can be made in a polypeptide sequence, and, of course, its underlying DNA coding sequence, and nevertheless obtain a polypeptide with functioanlly equivalent properties. It is thus contemplated by the inventors that various changes may be made in the peptide sequences of the disclosed compositions, or corresponding DNA sequences which encode said peptides without appreciable loss of their biological utility or activity. Functional equivalents and variants are used interchangably herein. In one preferred embodiment of the invention there is also provided variants of a regulator of morphology, and variants of fragments thereof.

The at least one regulator polypeptide in one embodiment is preferably one, wherein a substantially identical morphological shift is obtained from the production in a dimorphic fungal cell, under substantially identical conditions, of substantially identically amounts of i) the polypeptide comprising the at least one regulator of morphology of a dimorphic fungal cell, and ii) a functionally equivalent polypeptide comprising a functionally equivalent regulator of morphology, including any fragments thereof. A functionally equivalent polypeptide, or a fragment thereof, preferably comprises at least one conservative amino acid substitution. However, the invention is not limited to functional equivalents in the form of regulators comprising conservative substitutions.

When being polypeptides, variants are determined on the basis of their degree of identity or their homology with a predetermined amino acid sequence, said predetermined amino acid sequence being one of SEQ ID NO:2, SEQ ID NO:4, SEQ ID NO:6, SEQ ID NO:8, and SEQ ID NO:12, respectively, or, when the variant is a fragment, a fragment of any of the aforementioned amino acid sequences, respectively.

Accordingly, variants preferably have at least 75% sequence identity, for example at least 80% sequence identity, such as at least 85% sequence identity, for example at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with the predetermined sequence.

Sequence identity is determined in one embodiment by utilising fragments of regulator peptides comprising at least 25 contiguous amino acids and having an amino acid sequence which is at least 80%, such as 85%, for example 90%, such as 95%, for example 99% identical to the amino acid sequence of any of SEQ ID NO: 2, SEQ ID NO: 4, SEQ ID NO: 6, SEQ ID NO: 8, and SEQ ID NO:12, respectively, wherein the percent identity is determined with the algorithm GAP, BESTFIT, or FASTA in the Wisconsin Genetics Software Package Release 7.0, using default gap weights.

The following terms are used to describe the sequence relationships between two or more polynucleotides: “predetermined sequence”, “comparison window”, “sequence identity”, “percentage of sequence identity”, and “substantial identity”.

A “predetermined sequence” is a defined sequence used as a basis for a sequence comparison; a predetermined sequence may be a subset of a larger sequence, for example, as a segment of a full-length DNA or gene sequence given in a sequence listing, such as a polynucleotide sequence of SEQ ID NO:1 or SEQ ID NO:11, or may comprise a complete DNA or gene sequence. Generally, a predetermined sequence is at least 20 nucleotides in length, frequently at least 25 nucleotides in length, and often at least 50 nucleotides in length.

Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a predetermined sequence of at least 20 contiguous nucleotides and wherein the portion of the polynucleotide sequence in the comparison window may comprise additions or deletions (i.e., gaps) of 20 percent or less as compared to the predetermined sequence (which does not comprise additions or deletions) for optimal alignment of the two sequences.

Optimal alignment of sequences for aligning a comparison window may be conducted by the local homology algorithm of Smith and Waterman (1981) Adv. Appl. Math. 2: 482, by the homology alignment algorithm of Needleman and Wunsch (1970) J. Mol. Biol. 48: 443, by the search for similarity method of Pearson and Lipman (1988) Proc. Natl. Acad. Sci. (U.S.A.) 85: 2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected.

The homology between amino acid sequences may also be calculated using well known algorithms such as any one of BLOSUM 30, BLOSUM 40, BLOSUM 45, BLOSUM 50, BLOSUM 55, BLOSUM 60, BLOSUM 62, BLOSUM 65, BLOSUM 70, BLOSUM 75, BLOSUM 80, BLOSUM 85, and BLOSUM 90.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparision (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a predetermined sequence over a comparison window of at least 20 nucleotide positions, frequently over a window of at least 25-50 nucleotides, wherein the percentage of sequence identity is calculated by comparing the predetermined sequence to the polynucleotide sequence which may include deletions or additions which total 20 percent or less of the predetermined sequence over the window of comparison. The predetermined sequence may be a subset of a larger sequence, for example, a segment of any of the full-length SEQ ID NO:1 or SEQ ID NO:11 polynucleotide sequences illustrated herein.

As applied to polypeptides, a degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences.

An “unrelated” or “non-homologous” sequence shares less than 40% identity, though preferably less than 25% identity, with one of the regulator polypeptide sequences of the present invention. The term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions which are not identical differ by conservative amino acid substitutions.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine, a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

Additionally, variants are also determined based on a predetermined number of conservative amino acid substitutions as defined herein below. Conservative amino acid substitution as used herein relates to the substitution of one amino acid (within a predetermined group of amino acids) for another amino acid (within the same group), wherein the amino acids exhibit similar or substantially similar characteristics.

Within the meaning of the term “conservative amino acid substitution” as applied herein, one amino acid may be substituted for another within the groups of amino acids indicated herein below:

i) Amino acids having polar side chains (Asp, Glu, Lys, Arg, His, Asn, Gln, Ser, Thr, Tyr, and Cys,)

ii) Amino acids having non-polar side chains (Gly, Ala, Val, Leu, Ile, Phe, Trp, Pro, and Met)

iii) Amino acids having aliphatic side chains (Gly, Ala Val, Leu, Ile)

iv) Amino acids having cyclic side chains (Phe, Tyr, Trp, His, Pro)

v) Amino acids having aromatic side chains (Phe, Tyr, Trp)

vi) Amino acids having acidic side chains (Asp, Glu)

vii) Amino acids having basic side chains (Lys, Arg, His)

viii) Amino acids having amide side chains (Asn, Gln)

ix) Amino acids having hydroxy side chains (Ser, Thr)

x) Amino acids having sulphor-containing side chains (Cys, Met),

xi) Neutral, weakly hydrophobic amino acids (Pro, Ala, Gly, Ser, Thr)

xii) Hydrophilic, acidic amino acids (Gln, Asn, Glu, Asp), and

xiii) Hydrophobic amino acids (Leu, Ile, Val)

Accordingly, a variant or a fragment thereof according to the invention may comprise, within the same variant of the sequence or fragments thereof, or among different variants of the sequence or fragments thereof, at least one substitution, such as a plurality of substitutions introduced independently of one another.

It is clear from the above outline that the same variant or fragment thereof may comprise more than one conservative amino acid substitution from more than one group of conservative amino acids as defined herein above.

The addition or deletion of at least one amino acid may be an addition or deletion of from preferably 2 to 250 amino acids, such as from 10 to 20 amino acids, for example from 20 to 30 amino acids, such as from 40 to 50 amino acids. However, additions or deletions of more than 50 amino acids, such as additions from 50 to 100 amino acids, addition of 100 to 150 amino acids, addition of 150-250 amino acids, are also comprised within the present invention. The deletion and/or the addition may—independently of one another—be a deletion and/or an addition within a sequence and/or at the end of a sequence.

The polypeptide fragments according to the present invention, including any functional equivalents thereof, may in one embodiment comprise less than 250 amino acid residues, such as less than 240 amino acid residues, for example less than 225 amino acid residues, such as less than 200 amino acid residues, for example less than 180 amino acid residues, such as less than 160 amino acid residues, for example less than 150 amino acid residues, such as less than 140 amino acid residues, for example less than 130 amino acid residues, such as less than 120 amino acid residues, for example less than 110 amino acid residues, such as less than 100 amino acid residues, for example less than 90 amino acid residues, such as less than 85 amino acid residues, for example less than 80 amino acid residues, such as less than 75 amino acid residues, for example less than 70 amino acid residues, such as less than 65 amino acid residues, for example less than 60 amino acid residues, such as less than 55 amino acid residues, for example less than 50 amino acid residues.

Site-specific mutagenesis is a technique useful in the preparation of individual peptides, or biologically functional equivalent proteins or peptides, through specific mutagenesis of the underlying DNA. The technique, well-known to those of skill in the art, further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed.

The preparation of sequence variants of the selected peptide-encoding DNA segments using site-directed mutagenesis is provided as a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al. (1994); Segal (1976); Prokop and Bajpai (1991); and Maniatis et al.(1982), each incorporated herein by reference, for that purpose.

The PCR-based strand overlap extension (SOE) for site-directed mutagenesis is particularly preferred for site-directed mutagenesis of the nucleic acid compositions of the present invention. The techniques of PCR are well-known to those of skill in the art.

In one embodiment, functional equivalents or variants of a regulator of morphology will be understood to exhibit amino acid sequences gradually differing from the preferred predetermined regulator sequence, as the number and scope of insertions, deletions and substitutions including conservative substitutions increases. This difference is measured as a reduction in homology between the preferred predetermined sequence and the fragment or functional equivalent.

Accordingly, all fragments or functional equivalents of SEQ ID NO:2, SEQ ID NO:4, SEQ ID NO:6, SEQ ID NO:8, and SEQ ID NO:12, are included within the scope of this invention, regardless of the degree of homology that they show to the respective, predetermined regulator sequences disclosed herein. The reason for this is that some regions of the regulators are most likely readily mutatable, or capable of being completely deleted, without any significant effect on the binding activity of the resulting fragment.

A functional variant obtained by substitution may well exhibit some form or degree of native regulator activity, and yet be less homologous, if residues containing functionally similar amino acid side chains are substituted. Functionally similar in this respect refers to dominant characteristics of the side chains such as hydrophobic, basic, neutral or acidic, or the presence or absence of steric bulk. Accordingly, in one embodiment of the invention, the degree of identity is not a principal measure of a fragment being a variant or functional equivalent of a preferred predetermined fragment according to the present invention.

Fragments sharing homology with fragments of SEQ ID NO:2, SEQ ID NO:4, SEQ ID NO:6, SEQ ID NO:8, and SEQ ID NO:12, respectively, are to be considered as falling within the scope of the present invention when they are preferably at least about 90 percent homologous, for example at least 92 percent homologous, such as at least 94 percent homologous, for example at least 95 percent homologous, such as at least 96 percent homologous, for example at least 97 percent homologous, such as at least 98 percent homologous, for example at least 99 percent homologous with said predetermined fragment sequences, respectively. According to one embodiment of the invention the homology percentages refer to identity percentages.

Additional factors that may be taken into consideration when determining functional equivalence according to the meaning used herein are i) the ability of antisera to detect a functionally equivalent regulator of morphology according to the present invention, or ii) the ability of the functionally equivalent regulator of morphology to compete with a predetermined regulator of morphology in an assay.

In one embodiment, the sequence of immunogenically active amino acids within a known amino acid sequence is determined as described by Geysen in U.S. Pat. No. 5,595,915 which is incorporated herein by reference. A further suitably adaptable method for determining structure and function relationships of peptide fragments is described by U.S. Pat. No. 6,013,478, which is herein incorporated by reference. Also, methods of assaying the binding of an amino acid sequence to a receptor moiety are known to the skilled artisan.

In addition to conservative substitutions introduced into any position of a preferred predetermined regulator of morphology, or a fragment thereof, it may also be desirable to introduce non-conservative substitutions in any one or more positions of such a regulator.

A non-conservative substitution leading to the formation of a functionally equivalent fragment of regulator of morphology would for example i) differ substantially in polarity, for example a residue with a non-polar side chain (Ala, Leu, Pro, Trp, Val, Ile, Leu, Phe or Met) substituted for a residue with a polar side chain such as Gly, Ser, Thr, Cys, Tyr, Asn, or Gln or a charged amino acid such as Asp, Glu, Arg, or Lys, or substituting a charged or a polar residue for a non-polar one; and/or ii) differ substantially in its effect on polypeptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

Variants obtained by substitution of amino acids may in one preferred embodiment be made based upon the hydrophobicity and hydrophilicity values and the relative similarity of the amino acid side-chain substituents, including charge, size, and the like. Exemplary amino acid substitutions which take various of the foregoing characteristics into consideration are well known to those of skill in the art and include: arginine and lysine; glutamate and aspartate; serine and threonine; glutamine and asparagine; and valine, leucine and isoleucine.

The importance of the hydrophilic and hydropathic amino acid indices in conferring interactive biologic function on a protein is well understood in the art (Kyte & Doolittle, 1982 and Hopp, U.S. Pat. No. 4,554,101, each incorporated herein by reference).

It is accepted that the relative hydropathic character of the amino acid contributes to the secondary structure of the resultant polypeptide, which in turn defines the interaction of the polypeptide with other molecules, for example, binding partners and polynucleotides,

Accordingly, in a further embodiment the present invention relates to functional variants comprising substituted amino acids having hydrophilic values or hydropathic indices that are within +/−4.9, for example within +/−4.7, such as within +/−4.5, for example within +/−4.3, such as within +/−4.1, for example within +/−3.9, such as within +/−3.7, for example within +/−3.5, such as within +/−3.3, for example within +/−3.1, such as within +/−2.9, for example within +/−2.7, such as within +/−2.5, for example within +/−2.3, such as within +/−2.1, for example within +/−2.0, such as within +/−1.8, for example within +/−1.6, such as within +/−1.5, for example within +/−1.4, such as within +/−1.3 for example within +/−1.2, such as within +/−1.1, for example within +/−1.0, such as within +/−0.9, for example within +/−0.8, such as within +/−0.7, for example within +/−0.6, such as within +/−0.5, for example within +/−0.4, such as within +/−0.3, for example within +/−0.25, such as within +/−0.2 of the value of the amino acid it has substituted.

The amino acid hydropathic index values as used herein are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5) (Kyte & Doolittle, 1982).

The amino acid hydrophilicity values are: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4) (U.S. Pat. No. 4,554,101).

Production of a Desirable Gene Product by a Dimorphic Fungal Cell

The present fungal host cells can be used to express any prokaryotic or eukaryotic heterologous peptide or protein of interest, and is preferably used to express eukaryotic peptides or proteins. One particular interest for these species is their use in expression of heterologous proteins, such as enzymes and/or pharmaceutically useful polypeptides.

It will be understood by those skilled in the art that the term “enzymes and/or pharmaceutically useful polypeptides” includes not only native polypeptides, but also those polypeptides which have been modified by amino acid substitutions, deletions, additions, or other modifications, e.g. glycosylation, which may be made to enhance activity, thermostability, pH tolerance and the like.

However, the present host cells may also be used in recombinant production of proteins, which are native to the host cells. Examples of such use include, but are not limited to, i) placing a Mucor native gene under the control of a different promoter to enhance expression of the protein, ii) to expedite export of a native protein of interest outside the cell by use of a signal sequence, or iii) to increase copy number of a protein which is normally produced by the subject host cells. Thus, the present invention also encompasses, within the scope of the term “heterologous protein”, such recombinant production of homologous proteins, to the extent that such expression involves the use of genetic elements not native to the host cell, or use of native elements which have been manipulated to function in a manner not normally seen in the host cell.

The gene for the desired product functionally linked to promoter and terminator sequences may be incorporated in a vector containing the selection marker or may be placed on a separate vector or plasmid capable of being integrated into the genome of the host strain. The vector system may be a single vector or plasmid or two or more vectors or plasmids, which together contain the total DNA to be integrated into the genome. Vectors or plasmids may be linear or closed circular molecules.

According to one preferred embodiment of the present invention, the host is transformed with a vector comprising a selection marker, heterologous DNA to be introduced, including promoter, the gene for the desired protein and transcription terminator and polyadenylation sequences. Also the gene for the control of the dimorphic shift flanked by expression signals identical or different from the above allowing for regulated expression can be included.

The skilled artisan will recognize that the successful transformation of the host species described herein is not limited to the use of the vectors, promoters, and selection markers specifically exemplified. For example, although the leuA selection marker is preferred in some embodiments, other useful selection markers include, but are not limited to, pyrG, met and crgA, as well as markers conferring resistance to carboxin, oligomycin, geneticin (G-418), neomycin, kanamycin, zeocin, and hygromycin B.

As no multicopy vectors have been developed for zygomycetes and as among others M. circinelloides has been reported to be insensitive to various antibiotics such as geneticin (G418), neomycin, oligomycin and benomyl, (van Heeswijck et al., 1988), the use of corresponding antibiotic resistance genes as selection markers has so far been regarded as unfeasible in zygomycetes. However, the surprising results reported herein make it possible to construct replicons capable of being replicated in zygomycetes, including Mucorales, including Mucor, and maintaining cells comprising such replicons by selecting for antibiotic resistance.

A desirable feature of vectors useful for the analysis of gene expression and function is the ability to control the vector copy number. This can be achieved if the selective marker used confers a gene-dosage dependent phenotype. Such replicons can thus be used for cloning purposes. Examples of applications are enumerated below

Cloning of potential regulators of morphology under control of e.g. regulatable promoters such as the ones described herein. This opens the possibility for studying morphological pathways in vivo and for identifying further regulators of morphology.

Screening of genomic libraries, further cloning and identification of genes encoding industrial enzymes, secondary metabolites, etc. No expression libraries have been screened in zygomycetes to date. This may be due to the inherent difficulty in transforming this group of fungi (only a few species have been reported) and the inability to obtain a sufficiently high plasmid copy number in the few available transformation procedures for e.g., Mucor circinelloides. High frequency transformation using leucine selection in M. circinelloides permits the construction of representative libraries. The leuA gene was indeed cloned by direct screening of a M. circinelloides genomic library in strain R7B, a leuA mutant (van Heeswijck and Roncero, 1984). Using the system described herein (example 5), genomic libraries made from fungi such as non-transformable zygomycetes but also other fungi can be screened for. Subsequent geneticin selection of primary library clones renders a high plasmid copy number and contributes to enable the screening enzymatic activity or secondary metabolite production (vitamins, carotenes, antibiotics, etc).

In a related aspect, cDNA libraries can be constructed based on geneticin selection. Cloning of a strong or regulated promoter (e.g., from the M. circinelloides tubA or gal1 genes, respectively) into the multiple cloning site of vector pEUKA11 (FIG. 18) allows the directional cloning of cDNA fragments downstream of the promoter. As above, subsequent geneticin selection of primary library clones renders a high plasmid copy number, enabling the screening for different activities and metabolites. The use of a regulated promoter is preferred when screening for the production of toxic compounds to minimize deleterious effects.

The replicons may furthermore be capable of undergoing replication in other organisms such as e.g. E. coli. and the like. For this purpose the use of the expression cassette described in example 5 (gpd1P-kan) has been shown functional in E. coli, allowing for selection of transformants using kanamycin. Since this cassette is functional in E. coli and M. circinelloides, a series of shuttle vectors can be based in pEUKA11 and will permit the further cloning and characterization of clones identified during screening of libraries.

Another desirable feature of the replicons according to the present invention is that their copy number may be reflected by the concentration of antibiotics applied for selection. In this respect, amplification of genes may be obtained in M. circinelloides following the integration of an expression cassette containing the kan gene and a expression signal (promoter) into the M. circinelloides chromosome(s). Growing such a strain in medium containing high concentration of geneticin may lead to the amplification of the expression cassette. Adding a second expression cassette to the genetic construction design for integration of kan containing an expression signal and a gene of interest, improved production strains maybe obtained containing a higher copy number for the gene of interest. This procedure can be applied in strain improvement programs to obtain higher yields of recombinant proteins.

Such replicons can thus be used for cloning purposes, including the cloning of potential regulators of morphology under control of e.g. regulatable promoters such as the ones described herein. This opens the possibility for studying morphological pathways in vivo and for identifying further regulators of morphology. The replicons may furthermore be capable of undergoing replication in other organisms such as e.g. E. coli. and the like. Another desirable feature of the replicons according to the present invention is that their copy number may be reflected by the concentration of antibiotics applied for selection.

The present invention thus also embodies a method for selecting for antibiotic resistance in zygomycetes, including Mucorales, including Mucor species, including M. circinelloides, said method comprising the steps of providing a replicon comprising a selective marker in the form of an antibiotic resistance gene, and transforming said replicon into a zygomycete capable of harbouring said replicon, optionally comprising the further step of performing a cloning of a nucleotide sequence into said replicon prior to transformation. In another embodiment there is provided the use of an antibiotic resistance gene for selection for antibiotic resistance in zygomycetes.

The use of dominant (antibiotic resistance) markers is widespread in other fungi and yeast since the requirement for the isolation of auxotrophic strains is obviated. However, their use in zygomycetes has been constrained to two genus Absidia glauca and its fungal parasite Parasitella parasitica (Wostemeyer et al., 1987; Burmester 1992). For M. circinelloides, a naturally high resistance level was reported (van Heeswijck et al. 1988). The expression signals described herein such as gpd1P in combination with the selective marker of choice (e.g., kan) should be useful for transformation of natural isolates or industrial strains of zygomycetes which are elusive to classical methods for genetic analysis.

Exploiting the above results, the present invention also provides a dimorphic fungal cell comprising a replicon harbouring a selectable marker, e.g. an antibiotic resistance gene including a kanamycin resistance gene (kan), conferring resistance against an antibiotic, preferably geneticin in the case of kan. The replicon is preferably also capable of undergoing extrachromosomal replication in a bacterial cell such as E. coli. The replicon preferably comprises a regulatable promoter such as gpd1P (SEQ ID NO:9), or a fragment thereof capable of directing gene expression, operably linked to any nucleotide sequence of interest, for the purpose of expression of said nucleotide sequence of interest in said dimorphic cell. A constitutive promoter can also be employed. The replicon is also preferably capable of existing in the cell in different copy numbers, such as e.g. from about 0.5 copies per nucleus at a first predetermined concentration of a compound including antibiotica, and maintained at about 2 to 50 copies per nucleus at a second and higher predetermined concentration of a compound including antibiotica. Intermediate copy numbers per nucleus of about 5, 10, 15, 20, 25, 30, 35, 40, and 45 are also provided. The replicon in question preferably has a broad host range which makes it useful for transformation of e.g. zygomycetes including Mucorales. In further preferred embodiments the replicon comprises a transposable element which facilitates chromosomal integration. This may be linked to a thermosensitive replication origin which further facilitates chromosomal integration and/or amplification.

Regulatable Promoters

When there is provided a dimorphic fungal cell comprising

i) at least one nucleotide sequence encoding a desirable gene product, and operably linked thereto, and

ii) at least one further nucleotide sequence comprising a further expression signal capable of directing the expression in a dimorphic fungal cell of the at least one nucleotide sequence encoding the gene product, wherein said further expression signal is regulatable, during growth of the dimorphic fungal cell, by one or more of

a) the composition of the growth medium, including at least one of carbon source, nitrogen source including amino acids or precursors thereof, oxygen content, ionic strength, including NaCl content, pH, low molecular weight compounds, cAMP, and the presence or absence of a cell constituent, or a precursor thereof,

b) the temperature of the growth medium, including any change thereof, including an upshift eliciting the expression of one or more heat shock genes,

c) the growth phase of the dimorphic fungal cell, and

d) the growth rate of the dimorphic fungal cell.

wherein the nucleotide sequence encoding the gene product and the further nucleotide sequence comprising the regulatable expression signal are not natively associated,

the dimorphic cell preferably further comprises an isolated polynucleotide comprising

i) a first nucleotide sequence encoding at least one regulator of morphology capable of regulating the morphology of a dimorphic fungal cell, and operably linked thereto

ii) a second nucleotide sequence comprising an expression signal capable of directing the expression of the first nucleotide sequence in a dimorphic fungal cell,

wherein the first and second nucleotide sequences are not natively associated.

The dimorphic fungal cell preferably belongs to the class of Zygomycetes, including the order of Mucorales, including a genus selected from the group of genera consisting of Mucor, Thermomucor, Rhizomucor, Mycotypha, Rhizopus, and Cokeromyces, preferably the genus Mucor. Within the genus Mucor, the cell is preferably selected from the group of Mucor species consisting of M. circinelloides; M. hiemalis, M. rouxii, M. genevensis, M. bacilliformis, and M. subtillissimus, preferably M. circinelloides.

gpd1P

In one embodiment, the at least one further nucleotide sequence comprising the further expression signal is selected from the group consisting of

i) a polynucleotide comprising nucleotides 1 to 741 of SEQ ID NO:9,

ii) a polynucleotide comprising or essentially consisting of the promoter region of gpd1 of Mucor circinelloides, as deposited with DSMZ under accession number DSM 14066; and

iii) a polynucleotide comprising at least one fragment of SEQ ID NO:9, wherein said fragment

a) is capable of directing gene expression in a dimorphic fungal cell; and/or

b) is regulatable, during growth of the dimorphic fungal cell, by at least one factor capable of regulating gene expression directed by SEQ ID NO:9; and

iv) a polynucleotide, the complementary strand of which hybridizes, under stringent conditions, with a polynucleotide as defined in any of (i), (ii) and (iii), wherein said polynucleotide

a) is capable of directing gene expression in a dimorphic fungal cell; and/or

b) is regulatable, during growth of the dimorphic fungal cell, by at least one factor capable of regulating gene expression directed by SEQ ID NO:9,

and the complementary strand of such a polynucleotide.

Accordingly, there is provided in one embodiment at least one further nucleotide sequence comprising nucleotides 1 to 741 of SEQ ID NO:9.

In another embodiment there is provided a polynucleotide comprising or essentially consisting of the promoter region of gpd1 of Mucor circinelloides, as deposited with DSMZ under accession number DSM 14066.

In a still further embodiment, there is provided a polynucleotide comprising at least one fragment of SEQ ID NO:9, wherein said fragment

a) is capable of directing gene expression in a dimorphic fungal cell; and/or

b) is regulatable, during growth of the dimorphic fungal cell, by at least one factor capable of regulating gene expression directed by SEQ ID NO:9.

In a still further embodiment, there is provided a polynucleotide, the complementary strand of which hybridizes, under stringent conditions, with a polynucleotide as defined in any of (i), (ii) and (iii), wherein said polynucleotide

a) is capable of directing gene expression in a dimorphic fungal cell; and/or

b) is regulatable, during growth of the dimorphic fungal cell, by at least one factor capable of regulating gene expression directed by SEQ ID NO:9.

Fragments of SEQ ID NO:9 are preferably less than 500 nucleotides, such as less than 400 nucleotides, for example less than 350 nucleotides, for example less than 300 nucleotides, such as less than 250 nucleotides, for example less than 200 nucleotides, for example less than 150 nucleotides, such as less than 100 nucleotides. Preferred examples of fragments include, but is not limited to fragments of SEQ ID NO:9 comprising residues 22-741, 147-741, 270-741, 374-741, 507-741, and 683-741.

prnCP

There is also provided an embodiment, wherein the at least one further nucleotide sequence comprising the further expression signal is selected from the group consisting of

i) a polynucleotide comprising nucleotides 1 to 755 of SEQ ID NO:10,

ii) a polynucleotide comprising or essentially consisting of the promoter region of prnC of Mucor circinelloides, as deposited with DSMZ under accession number DSM 14067; and

iii) a polynucleotide comprising at least one fragment of SEQ ID NO:10, wherein said fragment

a) is capable of directing gene expression in a dimorphic fungal cell; and/or

b) is regulatable, during growth of the dimorphic fungal cell, by at least one factor capable of regulating gene expression directed by SEQ ID NO:10; and

iv) a polynucleotide, the complementary strand of which hybridizes, under stringent conditions, with a polynucleotide as defined in any of (i), (ii) and (iii), wherein said polynucleotide

a) is capable of directing gene expression in a dimorphic fungal cell; and/or

b) is regulatable, during growth of the dimorphic fungal cell, by at least one factor capable of regulating gene expression directed by SEQ ID NO:10,

and the complementary strand of such a polynucleotide.

Accordingly, there is provided in one embodiment at least one further nucleotide sequence comprising nucleotides 1 to 755 of SEQ ID NO:10.

In another embodiment there is provided a polynucleotide comprising or essentially consisting of the promoter region of prnC of Mucor circinelloides, as deposited with DSMZ under accession number DSM 14067.

In a still further embodiment, there is provided a polynucleotide comprising at least one fragment of SEQ ID NO:10, wherein said fragment

a) is capable of directing gene expression in a dimorphic fungal cell; and/or

b) is regulatable, during growth of the dimorphic fungal cell, by at least one factor capable of regulating gene expression directed by SEQ ID NO:10.

In a still further embodiment, there is provided a polynucleotide, the complementary strand of which hybridizes, under stringent conditions, with a polynucleotide as defined in any of (i), (ii) and (iii), wherein said polynucleotide

a) is capable of directing gene expression in a dimorphic fungal cell; and/or

b) is regulatable, during growth of the dimorphic fungal cell, by at least one factor capable of regulating gene expression directed by SEQ ID NO:10.

Fragments of SEQ ID NO:10 are preferably less than 500 nucleotides, such as less than 400 nucleotides, for example less than 350 nucleotides, for example less than 300 nucleotides, such as less than 250 nucleotides, for example less than 200 nucleotides, for example less than 150 nucleotides, such as less than 100 nucleotides.

tubAP

There is also provided an embodiment, wherein the at least one further nucleotide sequence comprising the further expression signal is selected from the group consisting of

i) a polynucleotide comprising nucleotides 1 to 927 of SEQ ID NO:13,

ii) a polynucleotide comprising or essentially consisting of the promoter region of tubA of Mucor circinelloides, as deposited with DSMZ under accession number DSM 14841; and

iii) a polynucleotide comprising at least one fragment of SEQ ID NO:13, wherein said fragment

a) is capable of directing gene expression in a dimorphic fungal cell; and/or

c) is regulatable, during growth of the dimorphic fungal cell, by at least one factor capable of regulating gene expression directed by SEQ ID NO:13; and

iv) a polynucleotide, the complementary strand of which hybridizes, under stringent conditions, with a polynucleotide as defined in any of (i), (ii) and (iii), wherein said polynucleotide

a) is capable of directing gene expression in a dimorphic fungal cell; and/or

b) is regulatable, during growth of the dimorphic fungal cell, by at least one factor capable of regulating gene expression directed by SEQ ID NO:13,

and the complementary strand of such a polynucleotide.

Accordingly, there is provided in one embodiment at least one further nucleotide sequence comprising nucleotides 1 to 927 of SEQ ID NO:13.

In another embodiment there is provided a polynucleotide comprising or essentially consisting of the promoter region of tubA of Mucor circinelloides, as deposited with DSMZ under accession number DSM 14841.

In a still further embodiment, there is provided a polynucleotide comprising at least one fragment of SEQ ID NO:13, wherein said fragment

a) is capable of directing gene expression in a dimorphic fungal cell; and/or

b) is regulatable, during growth of the dimorphic fungal cell, by at least one factor capable of regulating gene expression directed by SEQ ID NO:13.

In a still further embodiment, there is provided a polynucleotide, the complementary strand of which hybridizes, under stringent conditions, with a polynucleotide as defined in any of (i), (ii) and (iii), wherein said polynucleotide

a) is capable of directing gene expression in a dimorphic fungal cell; and/or

b) is regulatable, during growth of the dimorphic fungal cell, by at least one factor capable of regulating gene expression directed by SEQ ID NO:13.

Fragments of SEQ ID NO:13 are preferably less than 500 nucleotides, such as less than 400 nucleotides, for example less than 350 nucleotides, for example less than 300 nucleotides, such as less than 250 nucleotides, for example less than 200 nucleotides, for example less than 150 nucleotides, such as less than 100 nucleotides.

gal1P

There is also provided an embodiment, wherein the at least one further nucleotide sequence comprising the further expression signal is selected from the group consisting of

i) a polynucleotide comprising nucleotides 1 to 419 of SEQ ID NO:14,

ii) a polynucleotide comprising or essentially consisting of the promoter region of gal1 of Mucor circinelloides, as deposited with EMBL under accession number AJ438267; and

iii) a polynucleotide comprising at least one fragment of SEQ ID NO:14, wherein said fragment

a) is capable of directing gene expression in a dimorphic fungal cell; and/or

b) is regulatable, during growth of the dimorphic fungal cell, by at least one factor capable of regulating gene expression directed by SEQ ID NO:14; and

iv) a polynucleotide, the complementary strand of which hybridizes, under stringent conditions, with a polynucleotide as defined in any of (i), (ii) and (iii), wherein said polynucleotide

a) is capable of directing gene expression in a dimorphic fungal cell; and/or

b) is regulatable, during growth of the dimorphic fungal cell, by at least one factor capable of regulating gene expression directed by SEQ ID NO:14,

and the complementary strand of such a polynucleotide.

Accordingly, there is provided in one embodiment at least one further nucleotide sequence comprising nucleotides 1 to 419 of SEQ ID NO:14.

In another embodiment there is provided a polynucleotide comprising or essentially consisting of the promoter region of gal1 of Mucor circinelloides, as deposited with EMBL under accession number AJ438267.

In a still further embodiment, there is provided a polynucleotide comprising at least one fragment of SEQ ID NO:14, wherein said fragment

a) is capable of directing gene expression in a dimorphic fungal cell; and/or

b) is regulatable, during growth of the dimorphic fungal cell, by at least one factor capable of regulating gene expression directed by SEQ ID NO:14.

In a still further embodiment, there is provided a polynucleotide, the complementary strand of which hybridizes, under stringent conditions, with a polynucleotide as defined in any of (i), (ii) and (iii), wherein said polynucleotide

a) is capable of directing gene expression in a dimorphic fungal cell; and/or

b) is regulatable, during growth of the dimorphic fungal cell, by at least one factor capable of regulating gene expression directed by SEQ ID NO:14.

Fragments of SEQ ID NO:14 are preferably less than 400 nucleotides, such as less than 350 nucleotides, for example less than 300 nucleotides, for example less than 275 nucleotides, such as less than 250 nucleotides, for example less than 200 nucleotides, for example less than 150 nucleotides, such as less than 100 nucleotides.

The dimorphic fungal cell is preferably one, wherein the expression in said cell of the nucleotide sequence encoding the gene product results in the production of an increased amount of the gene product as compared to the production of the gene product in an at least substantially identical dimorphic fungal cell under substantially identical conditions, when the nucleotide sequence encoding the gene product is operably linked to the expression signal natively associated therewith.

The amount of the gene product produced is preferably increased by a factor of at least 1.02, for example by a factor of at least 1.05, such as a factor of at least 1.10, for example by a factor of at least 1.15, such as a factor of at least 1.20, for example by a factor of at least 1.25, such as a factor of at least 1.30, for example by a factor of at least 1.35, such as a factor of at least 1.40, for example by a factor of at least 1.45, such as a factor of at least 1.50, for example by a factor of at least 1.60, such as a factor of at least 1.70, for example by a factor of at least 1.80, such as a factor of at least 1.90, for example by a factor of at least 2.0, such as a factor of at least 2.5, for example by a factor of at least 3.0, such as a factor of at least 4.0, for example by a factor of at least 5.0, such as a factor of at least 7.5, for example by a factor of at least 10, such as a factor of at least 15, for example by a factor of at least 20, such as a factor of at least 25, for example by a factor of at least 50, such as a factor of at least 100, for example by a factor of at least 150, such as a factor of at least 200, for example by a factor of at least 250, such as a factor of at least 300, for example by a factor of at least 400, such as a factor of at least 500.

The at least one nucleotide sequence encoding the gene product and the operably linked to the at least one further nucleotide sequence comprising an expression signal are preferably located on a chromosomal replicon or an extrachromosomal replicon including an expression vector.

The nucleotide sequence encoding a gene product and/or the further nucleotide sequence comprising an expression signal is preferably derived from a fungal cell, including a dimorphic fungal cell, including a fungal cell belonging to the class of Zygomycetes, including a fungal cell belonging to the order of Mucorales, such as a genus selected from the group of genera consisting of Mucor, Mycotypha, and Cokeromyces, including the Mucor species consisting of M. circinelloides; M. rouxii, M. genevensis, M. bacilliformis, and M. subtillissimus.

The gene product is either homologous or heterologous to the dimorphic fungal cell, and the gene product may be located either in the cytoplasm of the dimorphic fungal cell or, when the nucleotide sequence encoding the gene product is e.g. preceded by a signal sequence encoding a signal peptide capable of mediating secretion of the gene product across the extracellular membrane of the dimorphic fungal cell, located in the growth medium following secretion.

To avoid the necessity of disrupting the cell to obtain the expressed product, and to minimize the amount of possible degradation of the expressed product within the cell, it is preferred that the product can be secreted outside the cell. To this end, in a preferred embodiment, the gene of interest is linked to a sequence encoding a signal peptide, which can direct the expressed product into the secretory pathway. The sequence encoding the signal peptide may be derived from any gene encoding a secreted protein from any organism, or may be part of the gene encoding the desired product. Among useful available sources for such a sequence encoding a signal peptide are genes encoding a glucoamylase, a lipase or a protease from Mucor racemosus or Rhizomucor miehei.

A signal peptide (or signal sequence) is an amino acid sequence which, when operably linked to the amino-terminus of a homologous or heterologous polypeptide, permits the secretion of such homologous or heterologus polypeptide from the host fungal cell. Signal peptides may be the signal peptide normally associated with the homologous or heterologous polypeptide (i.e., a native signal peptide), or it may be derived from other sources (i.e., a foreign signal peptide), or it may be synthetic. A signal peptide is operably linked to a homologous or heterologous polypeptide when the DNA sequence encoding a foreign or a native signal peptide is joined in the proper reading frame with a DNA sequence encoding the homologous or heterologous polypeptide to permit translation of the signal peptide and the homologous or heterologous polypeptide. Any amino acid sequence capable of permitting secretion of a polypeptide in a fungal host cell including a dimorphic fungal cell is contemplated by the present invention.

In addition to signal sequences, a DNA encoded precursor peptide may also be present. When positioned in the amino terminal end, such a precursor is known in the art as a propeptide or a prepropeptide. The precursor peptide may also be located in the carboxy terminal end, or in any other location of the mature homologous or heterologous polypeptide. When a precursor peptide is present, the resultant polypeptide is called a precursor polypeptide. In one embodiment, the precursor polypeptide is a zymogen. Zymogens are biologically inactive proteolytic enzymes and can be converted to mature active polypeptides by catalytic or autocatalytic cleavage of the precursor peptide from the zymogen.

A heterologous polypeptide is a polypeptide which is not normally expressed and secreted by the filamentous fungal host cell used to express that particular polypeptide. Heterologous polypeptides include polypeptides derived from prokaryotic sources (e.g., amylases from Bacillus species, alkaline proteases from Bacillus species, and various hydrolytic enzymes from e.g. Pseudomonas, etc.), polypeptides derived from eukaryotic sources (e.g., bovine chymosin, human tissue plasminogen activator, human growth hormone, human interferon, urokinase, human serum albumin, factor VIII etc.), and polypeptides derived from fungal sources other than the expression host (e.g., glucoamylase from A. niger and Humicola grisea expressed in A. nidulans, the carboxyl protease from Mucor miehei expressed in A. nidulans, etc.).

Heterologous polypeptides also include hybrid polypeptides which comprise a combination of partial or complete polypeptide sequences derived from at least two different polypeptides each of which may be homologous or heterologous with regard to the fungal expression host. Examples of such hybrid polypeptides include: 1) DNA sequences encoding prochymosin fused to DNA sequences encoding the A. niger glucoamylase signal and pro sequence alone or in conjunction with various amounts of amino-terminal mature glucoamylase codons, and 2) DNA sequences encoding fungal glucoamylase or any fungal carboxy protease, human tissue plasminogen activator or human growth hormone fused to DNA sequences encoding a functional signal sequence alone or in conjunction with various amounts of amino-terminal propeptide condons or mature codons associated with the functional signal.

The gene product is preferably selected from the group of gene products consisting of catalase, laccase, phenoloxidase, oxidase, oxidoreductases, cellulase, xylanase, peroxidase, lipase, hydrolase, esterase, cutinase, protease and other proteolytic enzymes, aminopeptidase, carboxypeptidase, phytase, lyase, pectinase and other pectinolytic enzymes, amylase, glucoamylase, alpha-galactosidase, beta-galactosidase, alpha-glucosidase, beta-glucosidase, mannosidase, isomerase, invertase, transferase, ribonuclease, chitinase, mutanase and deoxyribonuclease. Additionally preferred gene products are proteins and enzymes needed for processing of edible or drinkable products, antigens and vaccine components, therapeutic proteins, peptides and hormones.

### EXAMPLES

The following examples illustrate preferred embodiments of the present invention without limiting the invention to such embodiments. The below table lists the strains deposited with DSMZ.

| | | | | DSMZ |
|---|---|---|---|---|
| | OR- | VECTOR, | | ACCES- |
| | GAN- | GENETIC | RELEVANT | SION |
| STRAIN | ISM | MARKERS | FRAGMENT | NO. |
| pkaR13b- | E. coli | pCR2.1, Ap, Km | pkaR fragment | DSM14061 |
| 1 | | | (183 bp) | |
| pkaR1 | E. coli | YRp17, Ap | Full length pkaR | DSM14062 |
| | | | and promoter | |
| | | | (library clone) | |
| Fus3-4 | E. coli | pCR2.1, Ap, Km | mpk1 fragment | DSM14063 |
| | | | (541 bp) | |
| Ste12-2b | E. coli | pCR2.1, Ap, Km | ste12 fragment | DSM14064 |
| | | | (384 bp) | |
| UPO159 | E. coli | pCR2.1, Ap, Km | ste20 fragment | DSM14065 |
| | | | (600 bp) | |
| UPO160 | E. coli | pCR2.1, Ap, Km | gpd1P promoter | DSM14066 |
| | | | (740 bp) | |
| UPO129 | E. coli | pCR2.1, Ap, Km | prnCP promoter | DSM14067 |
| | | | (780 bp) | |
| UPO627 | M. | pEUKA4-gox1, | gpd1P-gox1- | DSM14068 |
| | circinel- | Ap, leuA, | trpCtt cassette | |
| | loides | | | |
| UPO842 | M. | pEUKA8-gox1, | prnCP-gox1- | DSM14069 |
| | circinel- | Ap, leuA | trpCtt cassette | |
| | loides | | | |
| UP263 | E. coli | pCR2.1TOPO, | Full length pkaC | DSM14839 |
| | | Ap, Km | and promoter | |
| MDO67 | E. coli | pCR2.1TOPO, | α-tubulin gene | DSM14841 |
| | | Ap, Km | (tubA) promoter | |
| | | | fragment | |

### Example 1

Direct Monitoring the Dimorphic Shift in Mucor circinelloides

The dimorphic fungal cell Mucor circinelloides is capable of undergoing a dimorphic shift when responding to environmental cues. The shift involves a change of morphology from e.g. a multinucleated cell having a unicellular, essentially spherical morphology, to e.g. a filamentous fungal cell characterised by an aseptate mycelium comprising multinucleated cells.

Following a shift from anaerobic to aerobic conditions, a transition gradually occurs from a unicellular, essentially spherical morphology to filamentous structures characterised by an aseptate mycelium comprising multinucleated cells occurs.

The present example illustrates the monitoring of this morphogenetic process on single cells.

Materials and Methods

M. circinelloides R7B (ATCC 90680), a leucine auxotrophic strain of M. circinelloides syn. racemosus (ATCC1216b) was used. Flask cultures were grown in YPG (complete medium) supplemented with 2% glucose. Anaerobic growth was achieved by bubbling a mixture of N₂/CO₂ (30%:70%) into the medium while stirring. During exponential growth, samples were taken into a 6-well microtiter plate and micrographs were taken on the same field at 10-min time intervals for 10 h using a microscope linked to a CCD camera.

Results

During anaerobic growth, M. circinelloides displays typical spherical and multipolar yeast growth (FIG. 1, panel 1). Upon shift to aerobic conditions a phase of growth in diameter is observed (FIG. 1, panels 2-4), representing the first 3 h after the shift. Subsequently, numerous protruding structures are visible which evolve into hyphae (FIG. 1, panels 7-8). Hyphae are structures that show a HGU of more than zero (value for yeast growth). Hyphal development (i.e., elongation) occurs rapidly. Branching of hyphae becomes evident and proliferates following the first 7 h after the shift (FIG. 1, panels 9-11).

### Example 2

Molecular Analysis of the Control of Dimorphism in Mucor circinelloides

The present example illustrates selected mechanisms underlying fungal dimorphism with emphasis on M. circinelloides.

Materials and Methods

Strains, Media and Cultivation.

M. circinelloides syn. racemosus R7B (ATCC 90680), a leucine auxotrophic strain of ATCC1216b, was used as a recipient for transformation experiments and for expression studies. Cultures were grown at 28° C. in either YPG (Bartnicki-Garcia and Nickerson, 1962), YNB (Lasker and Borgia, 1980), Vogel's medium supplemented with 1.5 g glutamic acid per litre (Hoekstra, 1996) or SIV medium with 1.5 g glutamic acid per litre in place of L-aspargine (Eslava and Alvarez, 1996). Vogel's and SIV media were supplemented with 2 g casamino acids (Difco, Detroit, Mich., USA) per litre. All media were supplemented with 1 mg/L niacin amide and 1 mg/L thiamine chloride. When appropriate, 20 mg/L leucine was added to allow for the growth of strain R7B. For the analysis of pkaR expression, 5 mM dbcAMP (Sigma) was added to aerobic growing cultures during exponential growth. The BBL GasPak system (Becton Dickinson, Sparks, Md., USA) was used for anaerobic incubation of strains on solid medium. E. coli DH5α was used for DNA manipulations.

Fermentation

Fermentation of M. circinelloides was carried out using a 2 liter bioreactor and ADI 1035 Bio Console (Applikon bioreactor systems, Applikon, The Netherlands). Cultures were inoculated with 10⁵ spores per ml. The pH was controlled during growth by the addition of 1 M NaOH. Under anaerobic growth conditions the culture was sparged with a mixture of 70% N₂/30% CO₂ (0.7 vvm), whereas aerobic conditions was achieved by sparging with atmospheric air (1.8 vvm).

DNA Manipulations

DNA isolation from M. circinelloides was carried using the FastDNA Spin kit for soil (BIO101 Inc, CA, USA) using frozen mycelium or yeast cells. All DNA manipulations were carried out according to the manufacturer's recommendations and standard protocols (Sambrook et al., 1989). Restriction enzymes, T4 DNA ligase, T4 polynucleotide kinase and other molecular biology reagents were from New England Biolabs, MA, USA or Life Technologies. Plasmid pCR2.1 (Invitrogen Corp.) was used for the cloning of PCR fragments.

PCR with Degenerate Primers, Inverse PCR and RT-PCR

PCR was carried out in a GeneAmp PCR Amplifier 2400 (Perkin Elmer, Foster City, Calif., USA), using Taq Polymerase (Life Technologies). For cloning of gene fragments using degenerate primers, genomic DNA isolated from R7B was used as template. For inverse PCR, genomic DNA of R7B was digested with appropriate enzymes and religated. PolyA mRNA was isolated from total RNA (see below) by oligotex (Qiagen) and used as template in RT-PCR using Omniscript Reverse Transcriptase (Qiagen). A 183-bp fragment of pkaR, pkaR13b-1, was obtained using the degenerate primers pkaR-1 and pkaR-3 (Table 1). This fragment was used as probe in colony hybridisation (see below). Subsequent approximately 500 bp of the promoter region was cloned by inverse PCR. Intron positions in pkaR were confirmed by sequencing of a fragment obtained by RT-PCR. A 369-bp fragment of pkaC was amplified using the degenerate primers pkaCfwd and pkaCrev (Table 1). The remaining 5′ part of the gene as well as approximately 500 bp of the promoter region was cloned in two rounds of inverse PCR. Additional 3′ sequence was obtained by RT-PCR using pkaCfwd and oligo-dT primers. The position of the first intron in pkaR was confirmed by sequencing of a fragment obtained by RT-PCR.

Plasmid Construction

The full-length coding region of the M. circinelloides pkaR gene spanning from the ATG start codon (position 1 for A in the obtained sequence) to the region down-stream of the polyadenylation signal (position 1418-1423) was amplified using primers pkaR-5′-EUKA4 and pkaR-3′-EUKA4 (spanning from position 1-25 and 1679-1707, respectively; Table 1). The obtained 1.7-kb fragment was cloned into pCR2.1, resulting in plasmid pCR1-pkaR, and transformed into E. coli. DNA of pCR1-pkaR was digested with NotI, purified from an agarose gel and partially digested with XhoI. The 1.7-kb full-length pkaR fragment was purified from an agarose gel and cloned into XhoI-NotI digested and phosphatase-treated pEUKA4 (EMBL accession number AJ305344) (Wolff and Arnau, 2002). In the resulting plasmid, named pEUKA4-pkaR, expression of pkaR is under control of the M. circinelloides gpd1 promoter (gpd1P), which is induced in response to the presence of glucose or galactose (Wolff and Arnau, 2002). As a vector control, plasmid pEUKA2 was used. This plasmid contains the same pUC13 backbone and wt leuA gene as pEUKA4-pkaR but lacks both gpd1P and the full length pkaR gene.

Transformation of M. circinelloides

Protoplasts formation and transformation was performed as previously described (van Heeswijck and Roncero, 1984) with the following modifications. Protoplasts were prepared by enzymatic treatment of germlings with a mixture of 125 μg chitosanase-RD (US Biological, MA) and 5 Units chitinase (from Streptomyces griseus, Sigma) in a final volume of 2 ml. Cell wall digestion was carried out for 2-3 h at 28° C. Typically, 1-10 μg DNA was used per transformation. Transformants were selected on YNB medium. Mucor transformant strains KFA121 and KFA89 were selected after transformation with pEUKA-pkaR and pEUKA2, respectively, amongst a few hundred transformants obtained in each case.

RNA Isolation and Analysis

RNA isolation was carried out using a FastRNA kit (BIO101 Inc., CA, USA) and extraction with acid Phenol:chloroform (Sigma). For Northern blot analysis, total RNA was loaded onto formaldehyde containing agarose gel, subjected to electrophoresis, transferred to GeneScreen membrane (Du Pont) and hybridised as described (Arnau et al., 1996). Expression levels were meassured using a Cyclone Storage Phosphor System (Packard) and the OptiQuant image analysis software. A linear dynamic range of 5 orders of magnitude with only a 5% standard deviation is possible with the above apparatus. Dilution series of the RNA preparations (typically 20, 10 and 5 μg total RNA per sample) were used to estimate induction levels. Primer extension was carried out using a radioactively end-labelled primer; complementary to position 98-78 downstream of the pkaR ATG start codon. A sequence ladder was obtained using the same primer and pEUKA4-pkaR DNA as template using the Thermo Sequenase Radiolabeled Terminator Cycle Sequencing Kit (USB-Amersham). Probe labelling was performed using Ready-To-Go DNA Labelling Beads (Amersham Pharmacia Biotech). Unincorporated nucleotides were removed using a NICK column (Pharmacia).

Genomic Library Screening

The M. circinelloides genomic library (Roncero et al., 1989) was used to screen for clones that contained pkaR sequences using the pkaR13b-1 fragment as a probe. Colony hybridisation was carried out as described (Sambrook et al., 1989).

DNA Sequencing, Sequence Analysis and Accession Numbers

DNA sequencing was performed in an ALF Express (Pharmacia) using Cy5 labelled primers as recommended. The DNASTAR package (Lasergene Inc., WI) was used for multi-alignments. The EMBL outstation Fasta3 and the NCBI Blast search programs were used for DNA and protein homology analysis. The sequence of the M. circinelloides pkaR, pkaC, mpk1, ste12 and ste20 genes described here have been deposited in the EMBL database with accession numbers AJ400723, AJ431364, AJ309731, AJ400724 and AJ309732, respectively.

TABLE 1
Primers used in this work

| PRIMER | Sequence (5′ to 3′) | |
|---|---|---|
| pkaR-1 | GGNGAYTAYTTYTAYGTNGTNGAR | (SEQ ID NO:15) |
| pkaR-3 | RAANGTNACNCKRTCNABNGCCCA | (SEQ ID NO:16) |
| pkaR5′-EUKA4 | ACTGGCTCGAGATGATCACTGACGAACATCCGTTTG | (SEQ ID NO:17) |
| pkaR3′-EUKA4 | ACGCTAGCGGCCGCCGCCTGCGCTTTGAGGTGGAGGCTCATC | (SEQ ID NO:18) |
| pkaCfwd | GGNAARGGNAGNTTYGGNCAR | (SEQ ID NO:19) |
| pkaCrev | RTTYTCNGGYTTNARRTCNCKRTA | (SEQ ID NO:20) |
| ste12-5′ | AARTTYTTYYTNGCNACNGCNCCNGTNAAYTGG | (SEQ ID NO:26) |
| ste12-3′ | RAACCARAARAANACYTTYTGYTTYTTYTGNGT | (SEQ ID NO:21) |
| mpk1-5′ | TAYATHGTNCARGARATHATG | (SEQ ID NO:22) |
| mpk1-3′ | CATDATYTCNGGNGCNCKRTACCA | (SEQ ID NO:23) |
| FUS3-3′ | CATDATYTCNGGNGCNCKRTACCA | (SEQ ID NO:23) |
| FUS3-5′ | TAYATHGTNCARGARATHATG | (SEQ ID NO:22) |

XhoI (CTCGAG) and NotI (GCGGCCGC) sites are shown in bold.

Results and Discussion

Cloning and Sequence Analysis of the M. circinelloides pkaR Gene

The protein sequences of several fungal regulatory subunits of protein kinase A (PKAR) are present in the public databases. The high level of sequence homology allowed the design of degenerate primers (Table 1) derived from the GDFFYVVE and WALDRNTS regions (positions 219-226 and 272-279 in the M. circinelloides PKAR protein sequence, see below) and the PCR amplification of a 183-bp fragment, named pkaR13b-1. Sequence analysis and database searches identified pkaR13b-1 as highly homologous to known fungal and eukaryotic PKAR encoding genes. Using pkaR13b-1 as a probe, a positive clone, pkaR1, was identified from a M. circinelloides genomic library. Sequence analysis of the 2-kb insert in pkaR1 showed that it contained a chromosomal insert encompassing the full-length M. circinelloides pkaR gene including 40-bp upstream of the ATG start codon. Further cloning using inverse PCR allowed the characterisation of the upstream region of pkaR including 541 bp of the promoter region (FIG. 3).

The promoter region (FIG. 3) includes two canonical CAAT boxes (positions −165 to −162 and −83 to −80, respectively), a putative TATA boxes (position −50 to −45 in the sequence) and a CT rich stretch (position −26 to −4) adjacent to the ATG start codon (position 1 for the A in the start codon).

The M. circinelloides pkaR gene includes two introns, one at the 5′-end of the coding region (position 203-254) and the other separating the coding region corresponding to the two cAMP-binding domains (position 1167-1219, see below). The pkaR gene encodes a putative 427 amino acid protein with an estimated molecular weight of 48.7 kDa. These data correlate with the estimated size for one of the identified protein species (51 kDa) in M. circinelloides using binding to radioactively labelled cAMP (Forte and Orlowski, 1980).

The M. circinelloides PKAR displays an overall homology to other fungal PKARs (31-45% identity, FIG. 4) and contains the expected well-conserved domains. Thus, two domains with a high degree of homology to cAMP-binding domains in other PKARs (94 to 64% identity), are present in the M. circinelloides PKAR (sFGELAL-mynAPRAATii and yFGELALIndAPRAATvv, at positions 247-264 and 369-386, respectively, in the amino acid sequence, FIG. 4). Further, a putative kinase inhibitor domain (RRTSVK) is found at position 144-149 in the amino acid sequence (FIG. 4). The partial sequence available from the PKAR of the related fungus M. rouxii does not include this domain (Sorol et al., 2000) and therefore comparison of the PKAR kinase inhibitor domain between these two Mucor species awaits.

Cloning and Sequence Analysis of the M. circinelloides pkaC Gene

Multiple alignment of several fungal protein sequences representing the catalytic subunit of protein kinase A (PKAC) reveals a high level of homology within the sequences corresponding to the ATP binding and the active sites. By PCR using degenerate primers (Table of primers) matching protein sequences within these regions (GKGTFGQ and YRNLKPES, respectively, see FIG. 5) a 369-bp fragment of M. circinelloides pkaC was obtained. The remaining parts of the gene as well as 534 bp of the promoter region were cloned by inverse PCR and RT-PCR.

The promoter region of pkaC includes five CAAT boxes (positions −430 to −427, −402 to −399, −387 to −384, −218 to −215 and −156 to −153, respectively) and a CT-rich region upstream (position −61 to −26) of the ATG start codon. Eventhough the promoter sequence contains some TA sequences no consensus TATA box was found.

The pkaC gene contains two putative introns in the 5′ end and putatively encodes a protein of 605 amino acid residues. This protein contains an ATP-binding domain (GQGSVG at position 254-259 in the amino acid sequence, FIG. 5) and a region with high homology to a serine/threonine protein kinase active site (position 367-379 in the amino acid sequence). Surprisingly, the conserved active site aspartic acid residue found in other PKACs is in M. circinelloides replaced by an aspargine residue. The codon encoding the aspargine residue (AAC) was confirmed by sequencing of different independent PCR products. However, we cannot completely rule out the possibility that this divergence is due to a PCR amplification artefact.

Expression of pkaR and pkaC in M. circinelloides during the Dimorphic Shift

As mentioned above, high levels of cAMP are associated in M. circinelloides with yeast growth and a rapid decrease in cAMP levels accompanies the transition from yeast to filamentous growth (Orlowski, 1991). These observations suggest that PKAC might be released and active during yeast growth, while PKAR repression might function during filamentous growth. In order to determine whether PKA in addition to regulation by cAMP is also regulated at the transcriptional level, expression of pkaR and pkaC under anaerobic yeast growth and aerobic filamentous growth was investigated by Norhern blot analysis. The expression of both pkaR and pkaC was found to be highly regulated. The expression levels of the two genes were higher in the anaerobic yeast culture than in the aerobic filamentous culture (FIG. 6A). The high level of pkaC expression during yeast growth (FIG. 6A, lane 3) correlates well with the hypothesis that PKA activity is essential for yeast growth.

Further, the level of pkaR expression (FIG. 6A, lane 1) can be explained by the necessity for the cell to be able to rapidly adapt to changes in environmental condition—thus, it is important that the PKAR is present and can bind and inactivate the PKAC upon fluctuations in cellular cAMP levels. During filamentous growth, the expression of pkaC is turned off (FIG. 6A, lane 4) indicating that PKA activity is not necessary during mycelial growth. The lack of pkaR expression under these conditions (FIG. 6A, lane 2) is an obvious advantage, since production of PKAR would be futile if PKAC is not present.

As described above, the levels of expression of pkaR and pkaC were found to be higher under anaerobic growth conditions as compared to aerobic growth conditions. These changes reflect the long-term adaptation to different environmental conditions. In order to determine whether inhibition exerted by PKAR is important during the dimorphic shift, the expression of pkaR during shift from yeast to filamentous growth was analysed by Northern blotting. An anaerobic M. circinelloides yeast culture was shifted to aerobic growth conditions. Four hours after the shift, filamentous growth was observed in the culture. The expression of pkaR was found to be higher after the shift to aerobic filamentous growth (FIG. 6B, lane 2) than during anaerobic yeast growth (FIG. 6B, lane 1). Quantification of mRNA levels indicated that a 2-fold induction of expression occurs during aerobic growth indicating that increased expression of pkaR shortly (4 hours) after the shift is necessary for the inhibition of PKA activity.

In Mucor, morphogenesis has been shown to be influenced by the availability of a fermentable carbon source and in S. cerevisiae, cAMP-dependent signal transduction is induced by glucose. To investigate if the expression of pkaR was regulated by glucose, anaerobic cultures shifted to aerobic growth with the simultaneous addition of glucose at different concentrations were analysed. No differences in expression levels were observed in the conditions used (0-10% (w/v) glucose), indicating that glucose does not alter pkaR expression levels during the shift to aerobic growth (FIG. 6B, lanes 3-5).

In a complementary study, the expression of pkaR was followed during aerobic (filamentous) growth before and after the addition of dbcAMP. This cAMP analogue is membrane permeable and its addition results in PKA activation. As shown in Table 2, pkaR expression is rapidly and gradually induced following dbcAMP addition.

TABLE 2
Induction of pkaR expression during aerobic growth in response to
dbcAMP

| Sample | Total Counts² | Fold induction³ |
|---|---|---|
| T:0¹ | 419 | 1.0 |
| T:15 min | 951 | 2.3 |
| T:30 min | 1728 | 4.1 |
| T:60 min | 2235 | 5.3 |
| T:120 min | 2392 | 5.7 |
| T:180 min | 2966 | 7.1 |

¹T:0: before the addition of 5 mM dbcAMP; T:15 min: 15 min after the addition, etc.
²Background counts were subtracted
³Values normalized with respect to counts at T:0 (1.0)

After three hours, a sevenfold induction was observed compared to the level observed before dbcAMP addition. Since aerobic growth is associated with a low level of PKA (or cAMP) in M. racemosus, the dramatic change in cAMP levels results in an immediate increase in pkaR expression. Under these conditions, the enhanced level of PKAR may counteract the excess cAMP during the period of transition from aerobic filamentous to yeast growth that follows after addition of dbcAMP to aerobic cultures (Orlowski 1991).

Overexpression of pkaR Leads to a Hyperbranching Phenotype

To investigate the role of PKAR during filamentous growth in M. circinelloides, a strain (KFA121) was constructed by transformation with plasmid pEUKA4-pkaR (FIG. 7A). Thus, KFA121 contains the pkaR gene under the control of the gpd1 promoter (gpd1P), recently isolated and characterised in our group (Wolff and Arnau, 2002). Primer extension experiments demonstrated that transcription of the plasmid-borne pkaR gene starts at the original transcription start site of the M. circinelloides gpd1P (FIG. 7A; Wolff and Arnau, 2002). The gpd1P is induced by glucose or galactose and a correlation between the concentration of sugar in the medium and the level of gpd1P-driven expression in M. circinelloides has been observed (Wolff and Arnau, 2002). Therefore, strain KFA121 was grown anaerobically in liquid YNB medium containing 5% glucose and the levels of expression of pkaR as well as pkaC were compared to the expression levels in a control strain (strain KFA89 obtained by transformation with pEUKA2) by Northern blot analysis. As seen, much higher levels of expression of pkaR and pkaC were observed in KFA121 as compared to the control strain grown under the same conditions (FIG. 7A). The increased expression of pkaC in the pkaR overexpressing strain indicates that a mechanism of co-regulation of pkaR and pkaC expression may exist. Furthermore, extensive degradation of mRNA was observed in KFA121. This observation may suggest that a regulatory mechanism is triggered in M. circinelloides KFA121 to prevent excess mRNA. In fact, specific RNA degradation has been reported in this fungus during heterologous expression of the E. coli GUS reporter (Garcia-Castillo et al., 2002).

No morphological differences were observed in aerobic liquid medium between KFA121 and the control strain. However, during growth on plates, KFA121 displayed a small colony phenotype. Microscopic examination of these colonies showed that a higher degree of branching at the hyphal tips occurred in KFAI21 (FIG. 7B). Taken together, these results indicate that PKAR plays a role in filamentation and branching in M. circinelloides during aerobic growth.

During anaerobic growth, the presence of non-germinated spores was observed for strain KFA121 both in solid and liquid medium (data not shown). Whether this is due to a titration of free PKAC subunits thereby hindering normal yeast growth remains to be examined.

Effect of Glucose on Morphology

As stated earlier, the level of various nutrients might be a major morphological determinant. In particular, a hexose is required in order to maintain anaerobic yeast growth. To determine the effect of glucose on morphology during anaerobic growth, M. circinelloides was incubated anaerobically on solid YNB medium with different glucose concentrations. At high glucose concentrations (0.5 to 5%), pure colony morphology with large yeast cells was maintained. However, growth on YNB with 0.1% glucose resulted in colonies displaying a centre of smaller yeast cells from which filamentous structures emerged (FIG. 8). It is likely that yeast growth is maintained until the glucose level reaches a minimum, thereby imposing a change in cell morphology via starvation.

The Transcription Factor STE12

STE12 is a transcription factor that participates in the MAPK-dependent signal transduction pathway in S. cerevisiae, C. albicans and C. neoformans leading to filamentation (Liu et al., 1994, Yue et al., 1999). In C. albicans, a ste12 null mutant strain is defective in filamentation (Liu et al., 1994). The STE12 transcription factor consists of a N-terminal region involved in DNA binding, a central induction domain and a C-terminal region involved in transcriptional activation. To investigate whether M. circinelloides possesses a ste12 homologue, PCR was carried out using degenerate primers designed from the most conserved sequence of the N-terminal region of available ste12 genes (KFFLATA and QKKQKVF, positions 44-50 and 151-157 in the S. cerevisiae STE12 protein sequence, FIG. 9A). A 384-bp fragment, ste12b-1, was obtained using R7B DNA as template. Sequence analysis revealed a high degree of homology between the protein sequence encoded by the ste12b-1 DNA sequence and other fungal STE12 homologues (56-64%, FIG. 9A), confirming that the cloned fragment is part of the M. circinelloides ste12 gene.

A MAP Kinase Homologue, mpk1 (Mitogen-activated Protein Kinase 1)

In S. cerevisiae and C. albicans, STE12 is activated by the MAP kinases KSS1 or CEK1, respectively, triggering filamentous growth. A homologous function should therefore exist in M. circinelloides. Two highly conserved regions present in the majority of cloned MAP kinases (YI/LVQEIMA and YRAPEIM; Lim et al., 1999; FIG. 9B) were chosen for the design of degenerate primers. A 541-bp fragment (mpk1-4) was amplified using R7B DNA as template. Sequence analysis showed that mpk1-4 potentially encodes a fragment of a M. circinelloides MAP kinase homologue and that three putative introns were present in the sequence obtained. The deduced protein sequence showed high identity to the Schizosaccharomyces pombe SPM1 and other fungal MAP kinases (66-73%, FIG. 9B). Identification of M. racemosus ste20 encoding a MAP kinase kinase kinase

In S. cerevisiae, STE20 participates in the MAP kinase signaling pathways involved in mating and pseudofilamentation. During the search for gene homologues to fungal hexokinases using degenerated primers, a clone (UPO896) was sequenced and the homology search identified it as a likely M. racemosus ste20 counterpart. The fragment cloned (634 bp) included sequence derived from one of the primers used at either end (data not shown) and spans two incomplete exons and an intron and the deduced protein sequence shows high homology to known STE20 and related serine/threonine kinases.

### Example 3

Use of M. circinelloides gpd1 for Recombinant Protein Production

The present example discloses three genes (gpd1, gpd2 and gpd3) encoding glyceraldehyde-3-phosphate dehydrogenase (GPD) and their isolation from the dimorphic zygomycete Mucor circinelloides by PCR using degenerated primers.

Only transcription of gpd1 could be detected during vegetative growth under both aerobic and anaerobic conditions, indicating that gpd1 is the main GPD-encoding gene. The transcription of gpd1 was significantly higher on fermentable carbon sources than on non-fermentable carbon sources during growth under aerobic conditions, indicating that gpd1 expression is subjected to carbon catabolite regulation.

A direct correlation between the abundance of gpd1 mRNA and the concentration of sugar in the medium was found during anaerobic growth. The gpd1 promoter was successfully used for recombinant expression of genes of both homologous (crgA) and heterologous (gox1 from A. niger) nature. Growth of a gox1 transformant strain resulted in the secretion of enzymatically active glucose oxidase.

Introduction

Genetic markers and a transformation system based on complementation of leucine auxotrophy have been established in M. circinelloides (Roncero et al., 1989). However, the availability of other genetic tools such as promoter sequences and alternative selection markers to allow for genetic studies and provide tools for recombinant protein production is very limited. In fact, only a few reports are published where Mucor has been used for heterologous protein production (Dickinson et al., 1987; Strøman et al., 1990).

Glyceraldehyde-3-phosphate dehydrogenase (GPD) is one of the key enzymes in the glycolytic pathway and in many eukaryotic microorganisms the GPD-encoding genes are expressed constitutively and at high levels (Holland and Holland, 1978; Edens, 1984; Waterham et al., 1997; Hirano et al., 1999). The promoter sequences of native GPD-encoding genes have proven useful for efficient expression of heterologous genes in several yeasts and fungi (Bitter and Egans, 1984; Waterham et al., 1997; Van den Hondel and Punt, 1991; Schuren and Wessels, 1994; Van de Rhee et a., 1996; Hirano et a., 2000). With the aim of obtaining a strong homologous promoter allowing efficient recombinant expression in M. circinelloides, GPD genes were cloned from M. circinelloides. The characterization of three individual genes (gpd1, gpd2 and gpd3) together with the use of the promoter region of the gpd1 gene for recombinant protein production is presented here.

Materials and Methods

Strains and Media

The Mucor circinelloides syn. racemosus strain R7B (ATCC 90680; a leucine auxotrophic derivative of ATCC 1216b) was used throughout this study. Mucor was grown in either YPG (Bartnicki-Garcia and Nickerson, 1962), YNB (Lasker and Borgia, 1980), Vogel's medium supplemented with 1.5 g glutamic acid pr. litre (Hoekstra et al., 1996) or SIV medium with 1.5 g glutamic acid pr. litre in place of L-aspargine and supplemented with 5 g casamino acids (DIFCO) pr. litre (Eslava and Alvarez, 1996). All media were supplemented with 1 mg/L niacin amide and 1 mg/L thiamine chloride. In some experiments glucose was replaced by galactose (20 g/L), glycerol (20 ml/L) or ethanol (5 ml/L). The E. coli strain Top10 (Invitrogen Corp.) was used in DNA manipulations and grown in LB medium (Sambrook et al., 1989).

Fermentation

Fermentation of M. circinelloides was carried out using a 2 liter bioreactor and ADI 1035 Bio Console (Applikon bioreactor systems, Applikon, The Netherlands). Cultures were inoculated with 5×10⁵ spores pr. ml. Strains were grown at 28° C., pH 5.0 and stirred at 200 rpm. Under aerobic growth conditions the culture was sparged with atmospheric air (0.5 vvm), whereas anaerobic conditions was achieved by sparging with 70% N₂/30% CO₂ (0.5 vvm). Biomass was determined as g cell dry weight pr. kg culture. Glucose in culture supernatant was determined using Glucose (HK) (Sigma Diagnostics).

PCR with Degenerate Primers and Inverse PCR (IPCR)

GPD-encoding genes were cloned using M. circinelloides R7B chromosomal DNA as template and degenerate primers designed from the well-conserved amino acid sequences: INGFGRIG and WYDNEYGY. PCR products of 1.1 kb were cloned in pCR2.1 (Invitrogen Corp.) and sequenced, and from the obtained DNA sequence primers for inverse PCR (IPCR) were designed. IPCR was performed as described (Ochman et al., 1990).

Colony Hybridization

A Mucor circinelloides genomic library constructed in the yeast-E. coli shuttle vector YRp17 was obtained from Professor M. I. G. Roncero (van Heeswijk and Roncero, 1984). E.coli clones were screened as described (Sambrook et al., 1989) using a gpd2-specific oligonucleotide as probe.

DNA Sequencing

Sequence reactions were carried out using cycle sequencing with fluorescent primers on an ALF Express sequencer (Pharmacia) according to the manufacturer's instruction. The obtained DNA sequences were deposited at EMBL (Accession nos. AJ293012, AJ293013 and AJ293014). The deduced amino acid sequences encoded by M. circinelloides gpd1, gpd2 and gpd3 were compared with those of GPD proteins from other fungi using the Clustal alignment feature of the MegAlign program (DNASTAR, Lasergene Inc., Madison, Wis.).

Construction of Expression Vector

Vectors based on pLeu4 (Arnau and Stroman, 1993) were constructed using standard techniques. The construction of pEUKA4 containing Ap^{R} and leuA as selection markers for selection in E. coli and M. circinelloides, respectively, the gpd1 promoter (nucleotides −741→−1 in FIG. 12) and the A. nidulans trpC terminator (EMBL Accession no. X02390, nucleotides 3563-4167) will be described elsewhere. The crgA gene (EMBL Accession no. AJ25099, nucleotides 626-2233) was amplified from M. circinelloides chromosomal DNA using PCR and the resulting fragment was cloned into the XhoI and NotI sites of pEUKA4 giving rise to pEUKA4-crgA. Similarly, a PCR generated fragment containing gox1 from A. niger (EMBL Accession no. X16061, nucleotides 40-1857) was cloned into pEUKA4 giving rise to pEUKA4-gox1. The complete nucleotide sequences of pEUKA4-crgA and pEUKA4-gox1 were deposited at EMBL (Accession nos. AJ305344 and AJ305345, respectively).

Primer Extension

Primer extension was performed using Primer Extension System (Promega) according to the manufacturers instruction. The gene specific primers gpd1primerREV (CATCCTTGTTGGACTCAGTAGC; SEQ ID NO:31), gpd2primerREV (CTTCAGGGTTAGAGAGAGAAGC; SEQ ID NO:32) and gpd3primerREV (CCTTGGGGTTTTCGAGGGAGG; SEQ ID NO:33) were used for primer extension. To determine the transcription start point a sequence reaction was performed using the same primers and run on the same gel as the primer extension products.

Northern Blot Analysis

Total RNA for Northern blot analysis was isolated using FastRNA and FastPrep FP120 (BIO101 Inc., CA, USA). Approximately 10 μg RNA was loaded onto formaldehyde containing agarose gel, subjected to electrophoresis and transferred to GeneScreen membrane (Du Pont) as described (Arnau et al., 1996) with the following modifications when oligonucleotide probes were used: i) Prehybridization and hybridization was performed at 50° C. and the buffer was supplemented with 1 M EDTA; ii) The blots were washed 1, 2 and 3 min at room temperature in 6×SSC, 1% SDS and 1.5 min at 50° C. in 1×SSC, 1% SDS. Probe labelling was performed using Ready-To-Go DNA Labelling Beads (Amersham Pharmacia biotech). Unincorporated nucleotides were removed using a NICK column (Pharmacia).

Zymogram

Proteins in culture supernatants or cell extracts were subjected to SDS-PAGE in 14% polyacrylamide gels under native conditions using precast gels (Novex, CA, USA). Cells were lysed using FastProtein Red and FastPrep FP120 (BIO101, Inc., CA, USA). After electrophoresis gels were incubated in the dark with assay reagent (0.1 M glucose, 0.1 mg/ml N-methyl-dibenzopyrazin methylsulphate (Sigma), 0.2 mg/ml 3-(4,5-dimethylthiazol-2-yl)2,5-diphenyltetrazolium bromid (Sigma) in 100 mM citrate-phosphate buffer pH 6.5) (Sock and Rohringer, 1988). Commercial GOX (Sigma) was used as positive control.

Transformation of M. circinelloides

Transformation of Mucor was performed basically as described (Van Heeswick et al., 1988). Protoplast formation was performed by digestion with 62.5 μg/ml chitosanase RD (US Biologicals, USA) and 12.5 units/ml chitinase (from Streptomyces griseus, Sigma).

Results and Discussion

Isolation of gpd Genes

Potential GPD-encoding genes were isolated from Mucor circinelloides by PCR using degenerate primers designed from highly conserved regions of GPD sequences from other fungi. The resulting PCR products of approximately 1.1 kb were cloned and sequencing of several independent clones revealed three different sequences (gpd1, gpd2 and gpd3) with significant homology to known GPD-encoding genes. The flanking DNA regions were obtained by inverse PCR (gpd1 and gpd3) or by colony hybridization (gpd2).

Introns

The genomic regions of M. circinelloides gpd1 (FIG. 10), gpd2 and gpd3 span 1127, 1213 and 1213 nucleotides, respectively, and contains two, three and two introns, respectively, with an average size of 63 nucleotides. All introns have 5′ splice site (GTA) and 3′ splice site (PyAG) in agreement with the consensus sequences for introns of filamentous fungi (Ballance, 1990). Further, the two introns in gpd1 and gpd3 and one of the three introns in gpd2 contain sequences similar to the “lariat formation” consensus sequence for filamentous fungi (5′-(G/A)CTAAC-3′) (Ballance, 1990). The introns in gpd1, gpd2 and gpd3 are placed at four different positions, i.e. three of the intron positions are conserved among two genes. It has previously been pointed out that the positions of introns are strongly conserved within the group of ascomycetes and basidiomycetes, respectively, but only the position of a single intron is conserved between the two classes (Harmsen et al., 1992). No introns have been found in GPD-encoding genes from ascomycetous yeasts. In contrast, the gpd genes from the basidiomycetous yeasts Phaffia rhodozyma and Cryptococcus neoformans contain 6 and 11 introns, respectively. A few of these introns are located at positions conserved among the filamentous basidiomycetes, but most of them are placed at unique positions, which may reflect the evolutionary divergence of the yeasts from the filamentous fungi. In M. circinelloides gpd1, gpd2 and gpd3, two of the four intron positions are also found among the basidiomycetes, whereas the other two positions are unique to the zygomycete which suggests closer phylogenetic relation of Mucor to basidiomycetes than to ascomycetes.

The 5′ and 3′ Flanking Regions

The sequences upstream of the coding regions of gpd1 (FIG. 10) and gpd2 contain consensus promoter elements within the expected distances relative to the ATG intiation codon. A putative TATA box (TATAAA) was observed 84 bp and 80 bp upstream of the initiating ATG of gpd1 and gpd2, respectively. Further, CAAT boxes were found 77 and 164 bp upstream of the start codon of gpd1 and 139 bp upstream of the start codon of gpd2. In contrast, these “core promoter” elements in the sequence upstream of gpd3 were found further upstream of the initiating ATG: a putative TATA box and a putative CAAT box were found 341 and 289 bp upstream of ATG, respectively. The site of transcriptional initiation of gpd1 was determined by primer extension to be 34 nucleotides upstream of the translation initiation codon. Primer extension using gpd2 and gpd3 specific primers did not result in any products neither with RNA isolated from an anaerobically growing yeast culture nor with RNA isolated from an aerobically growing filamentous culture, indicating that the gpd2 and gpd3 genes are not transcribed under these conditions. A pyrimidine region composed of stretches of thymine nucleotides interrupted by cytosine residues was observed immediately upstream of the transcription initiation site of gpd1 (FIG. 10) as has also been observed for other fungal genes (Gurr, 1988). Similar pyrimidine regions were also observed in the gpd2 and gpd3 sequences suggesting putative transcription initiation approximately 30 and 270 nucleotides upstream of ATG, respectively. Sequences conserved between the promoters of A. nidulans and A. niger GPD-encoding genes, such as gpd box, pgk box, qut box and qa box (Punt et al., 1990), are not present in the promoter regions of gpd1, gpd2 and gpd3. Consensus polyadenylation sites (AATAAA) were found 51 and 58 nucleotide after the stop codon of gpd1 and gpd2, respectively, whereas the same sequence was found 333 nucleotides downstream of the stop codon of gpd3. Overall, the three genes display nucleotide sequences required for transcription and subsequent processing within the 5′ and 3′ flanking regions. However, for gpd3 these sequence elements are found further away from the coding sequence than usually observed for fungal genes, which may result in sub-optimal transcription of gpd3 or even a non-functional gene.

Amino Acid Sequence and Codon Usage gpd1, gpd2 and gpd3 are predicted to encode polypeptides of 337, 338 and 339 amino acids, respectively. The three sequences show extensive homology to each other (80-86% identity) and to GPDs from other species (63-79% identity to known yeast and fungal GPDs). Previous phylogenetic analyses of yeast and fungal GPD sequences have shown clustering of GPDs from ascomycetous yeasts, filamentous ascomycetes and basidiomycetes, respectively, into distinct groups (Verdoes et al., 1997; Harmsen et al., 1992). As an exception the GPD encoded by gpd1 of the basidiomycete A. bisporus falls outside this grouping. The three M. circinelloides GPD sequences show highest homology to the sequences obtained from the group of basidiomycetes (>70% identity to the GPDs belonging to this group) and to a subset of the sequences obtained from the ascomycetous yeast (>70% identity to GPDs from S. pombe P. pastoris and C. albicans). In contrast, the homology to the GPD sequences obtained from the group of ascomycetes is lower: for most of the 15 GPD sequences in this group the identity is lower than 70%. A phylogenetic Clustal analysis showed grouping of the M. circinelloides sequences among the ascomycetous yeast sequences. Thus, amino acid sequence analysis of GPDs indicate a closer phylogenetic relation of Mucor to the basidiomycetes and a subset of the ascomycetous yeasts than to the filamentous ascomycetes. The codon usage in gpd1 and gpd2 is highly biased with 83% and 81% pyrimidines at the third position, respectively, and 21 unused codons in both genes. Pyrimidines dominate the third nucleotide position, but in contrast to many other fungal genes, U is preferred to C (Gurr et al., 1988). Where a purine is found in the third position, G is used in preference to A. The degree of codon bias in gpd3 is much lower with 67% pyrimidine at the third position and only 6 unused codons, suggesting that gpd3 is not a highly expressed gene.

Expression of gpd

The transcription of gpd1, gpd2 and gpd3 was analyzed under different growth conditions by Northern blotting using gene-specific oligonucleotides as probes derived from a divergent region. M. circinelloides R7B was grown in fermentor in Vogel's medium with either 2% or 5% glucose. Total RNA was isolated from a culture, growing exponentially as yeasts in the presence of glucose under anaerobic conditions, and from a culture, which had been shifted from anaerobic to aerobic conditions at the time of glucose depletion and further incubated for four hours allowing the initiation of filamentous growth. A high level of expression of gpd1 was observed in the yeast culture whereas low expression of gpd1 could be detected in the filamentous culture (FIG. 11). No expression of gpd2 or gpd3 was detected in any of the cultures. These data show that gpd1 is highly expressed under anaerobic growth conditions in presence of glucose and that the expression of gpd1 is highly regulated in response to either carbon source or anaerobiosis. Further, the data strongly indicate that although all three genes may potentially encode functional GPD, gpd1 is the major if not the only GPD-encoding gene in M. circinelloides as gpd2 and gpd3 are inactive at least during vegetative growth. In the cultivated/common mushroom Agaricus bisporus two genes have been identified, but only one of the two tandemly linked GPD genes is transcriptionally active (Harmsen et al., 1992). Conversely, in the budding yeast S. cerevisiae three separate GPD-encoding genes are differentially expressed, indicating that the different isoforms may have distinct cellular roles (Boucheriéet al., 1995). Whether the two genes in M. circinelloides, gpd2 and gpd3 represent functional genes which are only transcribed at specific growth stages (e.g. sporulation or germination), or non-functional pseudogenes remains to be shown.

The transcription of gpd1 was analyzed under various growth conditions by Northern blotting. M. circinelloides was grown aerobically in rich medium with different carbon sources in shake flasks. In the presence of glucose, a strong expression of gpd1 was observed while a significantly lower expression of gpd1 was observed when glycerol was present in the medium (FIG. 12A). Low expression of gpd1 was detected when ethanol was added to the medium. These results show that gpd1 expression is primarily regulated in response to carbon source rather than in response to aerobiosis. In order to analyze further the regulation of gpd1 expression in response to carbon source, M. circinelloides was grown as yeast under anaerobic conditions in rich medium with either glucose or galactose in fermentors. A strong expression of gpd1 was observed in the presence of high concentrations of glucose or galactose during growth in fermentor (FIG. 12B). The level of mRNA gradually decreased as the sugar was consumed and a low level of transcript was observed after depletion of sugar. However, subsequent addition of glucose to the culture resulted in a rapid increase in gpd1 expression (FIG. 12B, glucose concentration 1.4). These observations indicate that gpd1 expression in M. circinelloides is strongly regulated in response to sugar concentration. A slight (≦two-fold) decrease in mRNA level was observed during growth in fermentor under anaerobic conditions and after shift to aerobic conditions before glucose was depleted (data not shown). Thus, the transcription of gpd1 correlated with the concentration of glucose and galactose, whereas anaerobic versus aerobic growth conditions only had a minor effect on transcription. In many other filamentous fungi and yeasts e.g. A. nidulans, A. niger, N. crassa, K. lactis, P. pastoris and C. albicans expression of GPD-encoding genes has been found to be high and only subjected to minor changes during vegetative growth. Therefore, it was somewhat unexpected to find that the expression of the M. circinelloides gpd1 was highly regulated.

Recombinant Expression

It has previously been shown that recombinant expression of the crgA gene, encoding a regulator of carotene biosynthesis, can abolish the light requirement for carotene production in M. circinelloides (Navarro et al., 2000). In order to test whether the gpd1 promoter could mediate expression of the homologous gene crgA, the 740-bp gpd1 promoter region was placed upstream of the crgA gene in a M. circinelloides vector. The resulting plasmid (pEUKA4-crgA, FIG. 13A) was introduced into M. circinelloides strain R7B and transformants were incubated in the dark in order to prevent expression from the endogenous light-inducible crgA. After 3 days, crgA transformants showed a yellow-orange color, in contrast to a control strain, providing evidence that the gpd1 promoter indeed is able to drive expression of the crgA gene resulting in light-independent carotene synthesis (FIG. 13B).

In order to study heterologous gene expression in M. circinelloides, the gene encoding glucose oxidase (GOX) from Aspergillus niger (gox1, Frederick et al., 1990) was placed under the control of the gpd1 promoter in plasmid pEUKA4-gox1 (FIG. 13A). M. circinelloides R7B transformed with pEUKA4-gox1 was analyzed for GOX production in shake flask experiments. Analysis of culture supernatants by native gels/zymograms showed that active GOX was produced and secreted. The level of GOX activity correlated with the concentration of glucose in the growth medium, confirming that the gpd1 promoter is regulated in response to glucose concentration (FIG. 13C). M. circinelloides R7B transformed with pEUKA4-gox1 is deposited with DSMZ under Accession number DSM 14068.

The production of secreted GOX during anaerobic yeast growth, aerobic filamentous growth and transition from anaerobic yeast to aerobic filamentous growth was investigated in fermentation experiments. As seen (FIG. 14), active GOX accumulated in culture supernatants both under anaerobic and under aerobic growth conditions. The level of accumulated GOX was higher in the aerobically grown filamentous culture (FIG. 14C) as compared to the anaerobically grown yeast culture (FIG. 14A). However, the highest level of GOX accumulation was observed in the culture which initially had been grown as yeast under anaerobic conditions and then been shifted to aerobic conditions allowing filamentous growth (FIG. 14B). GOX secretion was confirmed by western blot analysis of supernatants from three cultures (FIG. 14D). These results indicate that a combination of a yeast growth phase and a filamentous growth phase favors the production and secretion of GOX in M. circinelloides.

In summary, three gpd homologues from the dimorphic zygomycete Mucor circinelloides have been cloned and characterised. Only expression of gpd1 was detected and the expression was found to be highly regulated in response to carbon source. The promoter of gpd1 was characterized and used for recombinant expression of the homologous gene crgA, encoding a regulator of carotene biosynthesis, and the heterologous gene gox1 from A. niger, encoding glucose oxidase. Recombinant expression of the regulatory crgA gene was evidenced phenotypically by the light independent formation of orange colonies of transformants carrying pEUKA4-crgA proving that it is possible to modify the regulation of a metabolic/biosynthetic pathway in M. circinelloides. Recombinant expression of A. niger gox1 resulted in production and secretion of enzymatically active GOX.

### Example 4

GOX Production during Dimorphic Shift in Mucor Using a Morphogenetically Regulated Promoter

The present example discloses expression of the gene encoding glucose oxidase (GOX) from Aspergillus niger (gox1, Frederick et al., 1990) directed by a M. circinelloides prnC promoter.

The plasmid, pEUKA8-gox1 (FIG. 15), was transformed into M. circinelloides R7B. M. circinelloides R7B transformed with pEUKA8-gox1 is deposited with DSMZ under Accession number DSM 14069.

Expression of prnC is induced during aerobic filamentous growth. The production of secreted GOX during anaerobic yeast growth, aerobic filamentous growth and transition from anaerobic yeast to aerobic filamentous growth was investigated in fermentation experiments. As seen in FIG. 16, active GOX accumulated in culture supernatants both under anaerobic and under aerobic growth conditions. However, the level of accumulated GOX was significantly higher during aerobic filamentous growth (FIGS. 16C and 16B after shift from anaerobic to aerobic conditions) as compared to yeast growth under anaerobic condition (FIGS. 16A and 16B before the shift from anaerobic to aerobic conditions).

These results indicate that expression of gox1 under the control of the prnC promoter in M. circinelloides is increased during aerobic filamentous growth, confirming the induction of the prnC promoter under these conditions.

### Example 5

A Vector System for the Screening of Genes Involved in the Regulation of Morphology of M. circinelloides

Overexpression of genes is widely used in yeast genetics as a approach to getting insights into fundamental aspects of yeast biology (Rine, 1991). Overexpression of genes can be achieved either by replacing the native promoter with a more powerful promoter or by increasing the gene copy number.

Hitherto no multicopy vectors have been developed for zygomycetes. Further, M. circinelloides has been reported insensitive to various antibiotics such as geneticin (G418), neomycin, oligomycin and benomyl, (van Heeswijck et al., 1988) precluding the use of the corresponding antibiotic resistance genes as selection markers.

However, the conditions used for the above screening—especially the acid pH used (3.2 or 4.5)—may result in the inactivation of the antibiotic. Therefore, we performed an initial screening of antibiotic sensitivity using pH 6.0. Under these conditions, M. circinelloides is sensitive to all four antibiotic tested. The most effective antibiotics were geneticin (no growth observed at 50 μg/ml) and hygromycin B (tiny colonies observed at 50 μg/ml and no growth at 100 μg/ml) (FIG. 17).

Here we describe the construction of a vector system, which allows for high plasmid copy number selection using a gene expression cassette conferring resistance to geneticin.

Geneticin is an aminoglycoside antibiotic, which interferes with the function of 80S ribosomes and blocks protein synthesis in eukaryotic cells. Genes encoding aminoglycoside phosphotransferases are present in bacterial transposons like Tn5 or Tn601 and can confer resistance to geneticin. The level of resistance is gene dosage dependent.

Construction of a vector, pEUKA7-kan was carried out by replacing the gox1 gene of pEUKA4-gox (Example 3, FIG. 13) with the coding region of the kanamycin resistance gene (kan) amplified by PCR from vector pCR2.1 (Invitrogen). Further, the Aspergillus nidulans trpC terminator present in pEUKA4-gox was replaced by the gpd1 terminator (FIG. 18A). Transformation of M. circinelloides strain R7B with pEUKA7-kan allow for selection of leucine prototrophy but also for geneticin selection.

During transformation of M. circinelloides, primary transformants are selected on selective medium at low pH, which promotes the formation of colonies with a small diameter. Selection at higher pH results in larger colony diameter and thereby confluent mycelial growth on plates making selection and propagation of numerous single isolates difficult. A pEUKA7-kan transformed strain, KFA143, was selected using leucine prototrophy and used to study the effect of geneticin selection on plasmid copy number in liquid culture. KFA143 was grown in YNB pH 6 (for leucine selection) and the same medium supplemented with 10 mg/L leucine and different concentrations of geneticin. Southern analysis using a leuA fragment as a probe, which hybridised both to the chromosomal leuA locus and to the plasmid-borne leuA, demonstrated that during leucine selection, the plasmid copy number is below one per genome (approx. 0.3 copies). Remarkably, during selection in medium with geneticin, the copy number increases to 20-30 copies per genome (FIG. 18B). At higher geneticin concentration, the plasmid copy number increases further, confirming the gene-dose dependent nature of resistance to this antibiotic.

We have therefore combined in a single vector the auxotrophic selection marker leuA allowing primary selection of numerous transformants and indeed useful for the direct cloning of genes from a genomic library (van Heeswijck and Roncero 1984) and an expression cassette containing the kan gene under the control of the M. circinelloides gpd1 promoter allowing subsequent selection for high plasmid copy number in medium containing geneticin.

Plasmid pEUKA7-kan is approx. 9-kb in size and therefore its use for genomic library construction maybe limited. Thus, in order to minimise the size of the vector, unnecessary DNA sequences were deleted from pEUKA7-kan. An AatII-SacI fragment between the ampicillin resistance gene (Ap) and the kanamycin resistance expression cassette, and an ApaI-MtuI fragment between the kanamycin resistance expression cassette and the leuA gene were deleted. A further fragment was deleted and a polylinker inserted between the leuA gene and the E. coli origin of replication by digestion with BstZ171 and partial digestion with PvuII. The resulting 7.2 kb vector, pEUKA11, contains the IeuA auxotrophic selection marker allowing selection of M. circinelloides transformants and the kanamycin resistance expression cassette allowing selection for high plasmid copy number as well as a polylinker containing multiple cloning sites (FIG. 18C). This vector will also be used to construct general-purpose expression libraries where cDNAs are cloned downstream of the selected promoter.

In a further refinement of the system, we established a procedure for the direct selection of transformants using pEUKA7-kan and geneticin. This selection combines plating the protoplast-DNA mixture on 20-ml plates containing 50 μg/ml geneticin (to reduce background growth normally obtained at pH 6) using a 10-ml top agar containing 125 μg/ml geneticin (i.e., final geneticin concentration of 75 μg/ml). Under these conditions, discrete transformant colonies were obtained using M. circinelloides R7B. The transformation frequency was somewhat lower than for leucine selection.

Geneticin selection can also be used to enable transformation of other related fungi where no auxotrophic strains are available. One such species is Mucor rouxii. Using pEUKA7-kan, M. rouxii transformants were obtained using the above selection procedure developed for M. circinelloides. The presence of the plasmid was confirmed using PCR with kan-specific primers (data not shown). Transformation of Rhizomucor pusillus strain B49-7 (Wada et al., 1996) with pEUKA7-kan was also achieved with both leucine and geneticin selection.

### Example 6

Expression of a Morphology Regulator in Heterologous Hosts

Expression of Mucor circinelloides pkaR in S. cerevisiae

A pkaR-cDNA clone was obtained by PCR using primers Pkar5′-kozak-SacI (ACTGCGGAGCTCATTATGATCACTGACGAACATCCG, SacI site underlined, SEQ ID NO:34) and PkaR3′-SphI (GCGCATGCTTATGATTGCTGGTTAATGAC, SphI site underlined, SEQ ID NO:35) and mRNA isolated from an anaerobically grown M. circinelloides culture as template. The expected 1.3-kb fragment was cloned into pCR2.1 and a positive clone (confirmed by sequencing) was digested with SacI and SphI. The purified pkaR DNA fragment was ligated to pYES2 digested with the same restriction enzymes, resulting in pYES-pkaR. The pYES vector system allows the study of regulated expression in yeast via the GAL1 promoter. Expression is induced in galactose-containing medium and repressed in the presence of glucose. S. cerevisiae strain PLFY104 (Mat-α ura3-52) was chosen as a recipient since this genetic background permits the examination of pseudohyphal growth in the haploid phase (Cullen and Sprague, 2000).

Strain PLFY104 was transformed by electroporation with pYES-pkaR and transformants were selected in medium without uracil. A strain named MDO41 was selected among the transformants. As a control, transformation of PLFY104 with the vector pYES2 was carried out and a strain, MDO39, was selected.

Pseudohyphal differentiation is triggered upon starvation in S. cerevisiae. Addition of nutrients to stationary cells results in a dramatic increase in the levels of protein kinase A and the resumption of growth (Robertson et al., 2000).

Growth of MDO41 was investigated in liquid medium. Cells were grown to stationary phase in galactose medium and a 50-fold dilution was inoculated in fresh medium with either glucose or galactose and raffinose as carbon sources. As shown (FIG. 19), a slower growth rate was observed for MDO41 compared to MDO39 in galactose/raffinose medium, indicating that the expression of M. circinelloides pkaR results in repression of the yeast protein kinase A encoded by the TPK genes.

In order to investigate the effect of overexpression of pkaR on morphology in yeast, strain MDO39 and MDO41 were grown overnight in minimal defined medium. Subsequently, a dilution of the culture was grown in liquid SC medium with galactose and raffinose for 5 h (induction medium) and plated on to plates with glucose (control) or plates with galactose/raffinose. In glucose, yeast colony morphology was observed for both strains (FIG. 20, top). A drop of 10% (w/v) glucose was added immediately after plating to the plates with galactose/raffinose and incubated further for 4 hours.

Microscopical examination revealed that, at the position of glucose addition, round yeast colonies were observed for MDO39 and MDO41, although limited pseudohyphal structures were also evident in MDO41 at this position (FIG. 20). Pseudohyphal morphology was more noticeable for MDO41 at locations close to the position of the glucose drop and the extent of pseudohyphal growth increased with the distance from the position of glucose addtion. Limited pseudohyphal morphology was observed for MDO39 exclusively at positions away from the glucose drop, suggesting that galactose and raffinose might trigger limited nutritional stress in the PLFY104 background.

MDO41 showed more extensive pseudohyphal growth even close to the position of glucose addition demonstrates that overexpression of pkaR in S. cerevisiae can be used to modulate pseudohyphal growth and thereby morphology.

Expression of Mucor circinelloides pkaR in Rhizomucor pusillus

Rhizomucor pusillus is a non-dimorphic zygomycete of industrial relevance. A transformation system has been established using leucine selection in a manner similar to the leuA selection in Mucor circinelloides (Wada et al., 1996). Using the pEUKA4-pkaR and pEUKA4-crgA vectors which both contain the M. circinelloides leuA gene as a selective marker, we transformed R. pusillus strain B49-7. Single transformants named KFA183 (for crgA) and KFA185 (for pkaR) were selected and the presence of plasmid was confirmed by PCR using crgA- and pkaR-specific primers, respectively (data not shown).

Analysis of growth on plates showed a longer lag phase for KFA185 compared to KFA183 in defined, selective medium. The crgA gene has been characterized as a repressor of carotene biosynthesis in M. circinelloides (Navarro et al., 2001). Strain KFA183 displayed a pale colony color due to the lack of carotenes, confirming that expression of the heterologous crgA indeed occurs in R. pusillus and that the M. circinelloides CrgA is able to repress carotene synthesis in R. pusillus. Moreover, these results demonstrate that the promoter of the M. circinelloides gpd1 gene is functional in other zygomycetes.

To study the effect of pkaR overexpression in R. pusillus on colony morphology, strain KFA183 and KFA185 were grown in YNB medium with either 1 or 5% glucose. In medium with 1% glucose, both strains showed a similar degree of branching (FIG. 21). Remarkably, in 5% glucose a higher degree of branching and a more compact colony morphology was observed for KFA185 compared to KFA183 (FIG. 21), demonstrating than heterologous overexpression of M. circinelloides pkaR in R. pusillus has an effect in colony morphology.

These results indicate that colony morphology of different fungal and yeast species can be modulated by heterologous expression of pkaR.

### Example 7

Isolation and Analysis of Alpha-tubulin Promoter

The high level of sequence homology between α-tubulin proteins from various organisms allowed the design of degenerate primer derived from the EHGIQPDG and WYVGEGM sequences. A 1,2 kb DNA fragment containing the majority of the M. circinelloides gene encoding α-tubulin was cloned by PCR using the degenerate primers tba1fwd2 (GARCAYGGNATHCARCCNGAYGG; SEQ ID NO:29) and tba1rev2 (CATNCCYTCNCCNACRTACCA; SEQ ID NO: 30). Additional upstream sequence was cloned in two rounds of inverse PCR resulting in 927 bp upstream of the start ATG codon of the α-tubulin gene (tubA). The expression of tubA was analysed by Northern blotting using RNA isolated from an anaerobic yeast culture and an aerobic filamentous culture respectively. The gene was found to be strongly expressed under both growth conditions.

### Example 8

Isolation and Analysis of Galactokinase Promoter

With the aim of obtaing a M. circinelloides promoter which is regulated in response to carbon-source a fragment of an M. circinelloides gene encoding galactokinase was cloned by PCR using degenerate primers derived from the well-conserved sequences PGRVNLIG and TGAGWGG, respectively. Primers used for cloning were Gal1fwd3 (CCNGGNMGNGTNAAYYTNATHGG; SEQ ID NO: 27) and Gal1rev1 (CCNCCCCANCCNGCNCCNGT; SEQ ID NO: 28). Subsequently, 420 bp of the sequence upstream of the start ATG codon of the galactokinase gene (gal1) was cloned by inverse PCR (SEQ ID NO:14, submitted to EMBL under accession number AJ438267). The expression of gal1 was analysed by Northern blotting using RNA isolated from cultures grown in rich medium containing various carbon sources. Without any carbon source added (lane 5) or with the addition of glycerol (lane 4) to the growth medium a very low level of expression of gal1 was observed. In medium containing galactose a high expression level (lane 2) was detected. In the presence of glucose either alone (lane 1) or in combination with galactose (lane 3) significantly lower expression levels were observed as compared to the level observed when galactose was added alone. These results indicate that the expression of gal1 is induced by galactose and partially repressed by glucose.

References

Arnau, J., Sørensen, K. I., Appel, K. F., Vogensen, F. K. and Hammer, K. 1996. Analysis of heat shock gene expression in Lactococcus lactis MG1363. Microbiology 142: 1685-91.

Arnau, J. and Strøman, P. 1993. Gene replacement and ectopic integration in the zygomycete Mucor circinelloides. Curr. Genet. 23: 542-6.

Balance, D. J. 1990. Transformation systems for filamentous fungi and an overview of fungal gene structure. In Molecular Industrial Mycology (S. A. Leong and R. M. Berka, Eds.), pp. 1-29. Marcel Dekker, NY.

Bartnicki-Garcia, S. and Nickerson, W. J. 1962. Induction of yeastlike development in Mucor by carbon dioxide. J. Bacteriol. 84: 829-40.

Bitter, G. A. and Egan, K. M. 1984. Expression of heterologous genes in Saccharomyces cerevisiae from vectors utilizing the glyceraldehyde-3-phosphate dehydrogenase gene promoter. Gene 32: 263-74.

Bocking, S. P., Wiebe, M. G., Robson, G. D., Hansen, K., Christiansen, L. H. and Trinci, A. P. 1999. Effect of branch frequency in Aspergillus oryzae on protein secretion and culture viscosity. Biotechnol. Bioeng. 65: 638-48

Borges-Walmsley, M. I. and Walmsley, A. R. 2000. cAMP signalling in pathogenic fungi: control of dimorphic switching and pathogenicity Trends Microbiol. 8: 133-41.

Boucherié, H., Bataille, N., Fitch, I. T., Perrot, M. and Tuite, M. F. 1995. Differential synthesis of glyceraldehyde-3-phosphate dehydrogenase polypeptides in stressed yeast cells. FEMS Microbiol. Lett. 125: 127-34.

Chaudhuri A, Bharadwaj G, Maheshwari R. 1999. An unusual pattern of invertase activity development in the thermophilic fungus Thermomyces lanuginosus. FEMS Microbiol Lett. 177:39-45.

Cullen, P. J. and Sprague, G. F. 2000. Glucose depletion causes haploid invasive growth in yeast. Proc. Natl. Acad. Sci., USA 97: 13619-24.

Dickinson, L., Harboe, M., van Heeswijck, R., Strøman, P. and Jepsen, L. P. 1987. Expression of active Mucor miehei aspartic protease in Mucor circinneloides. Carlsberg Res. Commun. 52: 243-52.

Ernst, J. F. 2000. Regulation of dimorphism in Candida albicans. Contrib.Microbiol. 5: 98-111.

Eslava, A. P. and Alvarez, M. I. 1996. Genetics of Phycomyces. In Fungal genetics—Principle and practice (J. B. Cees, Ed.), Mycology series vol. 13, pp. 385-406. Marcel Dekker, New York.

Forte, J. W. and Orlowski, M. 1980. Profile of cyclic adenosine 3′,5′-monophosphate binding proteins during the conversion of yeast to hyphae in the fungus Mucor. Exp. Mycol. 4: 78-86.

Frederick, K. R., Tung, J., Emerick, R. S., Masiarz, F. R., Chamberlain, S. H., Vasavada, A., Rosenberg, S., Chakraborty, S., Schopfer, L. M. and Massey, V. 1990. Glucose oxidase from Aspergillus niger. Cloning, gene sequence, secretion from Saccharomyces cerevisiae and kinetic analysis of a yeast-derived enzyme. J. Biol. Chem. 265: 3793-802.

Garcia-Castillo, J., Lorca-Pascual, J. M., Torres-Martinez, S. and Ruiz-Vazquez, R. 2000. Expression of the reporter gene uidA in Mucor circinelloides is abolished by premature termination of transcription. In ECFG5 Abstract Book (B. Felenbok and B.Turcq, Eds.)

Gordon, C. L., Khalaj, V., Ram, A. F., Archer, D. B., Brookman, J. L., Trinci, A. P., Jeenes, D. J., Doonan, J. H., Wells, B., Punt, P. J., van den Hondel, C. A. and Robson, G. D. 2000. Glucoamylase::green fluorescent protein fusions to monitor protein secretion in Aspergillus niger. Microbiology. 146: 415-26.

Gurr, S. J., Unkles, S. E. and Kinghorn, J. R. 1988. The structure and organisation of nuclear genes of filamentous fungi. In Gene Structure in Eukaryotes (J. R. Kinghorn, Ed.), pp. 93-139. IRL Press, Oxford.

Harmsen, M. C. Schuren, F. H. J., Moukha, S. M., van Zuilen, C. M., Punt, P. and Wessels, G. H. 1992. Sequence analysis of the glyceraldehyde-3-phosphate dehydrogenase genes from the basidiomycetes Schizophyllum commune, Phanerochaete chrysosporium and Agaricus bisporus. Curr. Genet. 22: 447-54.

Hirano, T., Sato, T., Okawa, K., Kanda, K., Yaegashi, K. and Enei, H. 1999. Isolation and characterization of the glyceraldehyde-3-phosphate dehydrogenase gene of Lentinus edodes. Biosci. Biotechnol. Biochem. 63: 1223-27.

Hirano, T., Sato, T. Yaegashi, K. and Enei, H. 2000. Efficient transformation of the edible basidiomycete Lentinus edodes with a vector using a glyceraldehyde-3-phosphate dehydrogenase promoter to hygromycin B resistance. Mol. Gen. Genet. 263: 1047-52.

Hoekstra, R. F. 1996. Horizontal transmission in fungal populations. In Fungal genetics—Principle and practice (J. B. Cees, Ed.), Mycology series vol. 13, pp. 337-348. Marcel Dekker, New York.

Holland, M. J. and Holland, J. P. 1978. Isolation and identification of yeast messenger ribonucleic acids coding for enolase, glyceraldehyde-3-phosphate dehydrogenase, and phosphoglycerate kinase. Biochemistry 17: 4900-7.

Iturriaga, E. A., Diaz-Minguez, J. M., Benito, E. P., Alvarez, M. I. and Eslava, A. P. 1992. Heterologous transformation of Mucor circinelloides with the Phycomyces blakesleeanus leu1 gene. Curr. Genet. 21: 215-23.

Lasker, B. A. and Borgia, P. T. 1980. High-frequency heterokaryon formation by Mucor racemosus. J. Bacteriol. 141: 565-9.

Liu, H., Kohler, J. and Fink, G. R. 1994. Suppression of hyphal formation in Candida albicans by mutation of a STE12 homolog. Science 266: 1723-26.

Madhani, H. D. and Fink, G. R. 1998. The control of filamentous differentiation and virulence in fungi. Trends Cell Biol. 8: 348-53.

Moreno, S. and Passeron, S. 1980. Further studies on cyclic adenosine 3′:5′-monophosphate protein kinase from dimorphic fungus Mucor rouxii. Arch. Biochem. Biophys. 199:321-30.

Navarro, E., Lorca-Pascual, J. M., Quiles-Rosillo, M. D., Nicolas, F. E., Garre, V., Torres-Martinez, S. and Ruiz-Vazquez, R. M. 2001. A negative regulator of light-inducible carotenogenesis in Mucor circinelloides. Mol. Genet. Genomics 266: 463-70.

Navarro, E., Ruiz-Pérez, V. L. and Torres-Martinez, S. 2000. Overexpression of the crgA gene abolishes light requirement for cartenoid biosynthesis in Mucor circinelloides. Eur. J. Biochem. 267: 1-10.

Ochman, H., Medhora, M. M., Garza, D. and HartI, D. L. 1990. Amplification of flanking sequences by inverse PCR. In PCR protocols: A guide to methods and applications (M. A. Innis, Ed.), pp. 219-27. Academic Press, San Diego.

Orlowski, M. 1991. Mucor dimorphism. Microb. Rev. 55: 234-58.

Pi, H., Chien, C. T., Fields, S. 1997: Transcriptional activation upon pheromone stimulation mediated by a small domain of S. cerevisiae Ste12p. Mol. Cel. Biol. 17: 6410-18.

Punt, P. J., Dingemanse, M. A., Kuyvenhoven, A., Soede, R. D. M., Pouwels, P. H. and van den Hondel, C. A. M. J. J. 1990. Functional elements in the promoter region of the Aspergillus nidulans gpdA gene encoding glyceraldehyde-3-phosphate dehydrogenase. Gene 93: 101-9.

Rine, J. 1991. Gene overexpression in studies of Saccharomyces cerevisiae. Methods Enzymol. 194: 239-51.

Roberts, R. L. and Fink, G. R. 1994. Elements of a single MAP kinase cascade in Saccharomyces cerevisiae mediate two developmental programs in the same cell type: mating and invasive growth. Genes Dev. 8: 2974-85.

Robertson, L. S., Causton, H. C., Young, R. A. and Fink, G. R. 2000. The yeast A kinases differentially regulate iron uptake and respiratory function. Proc Natl Acad Sci USA. 97: 5984-8.

Roncero, M. I., Jepsen, L. P., Strøman, P. and van Heeswijck, R. 1989. Characterization of a leuA gene and an ARS element from Mucor circinelloides. Gene 84: 335-43.

Rossi, S. and Moreno, S. 1994. Regulation of protein kinase A subunits during germination of Mucorrouxii sporangiospores Eur.J Biochem. 222: 501-6.

Roze, L. V., Mahanti, N., Mehigh, R., McConnell, D. G., and Linz, J. E. 1999. Evidence that MRas1 and MRas3 proteins are associated with distinct cellular functions during growth and morphogenesis in the fungal cell Mucor racemosus Fungal. Gen. Biol. 28: 171-89.

Ruiz-Hidalgo, M. J., Eslava, A. P., Alvarez, M. I. and Benito, E. P. 1999. Heterologous expression of the Phycomyces blakesleeanus phytoene dehydrogenase gene (carB) in Mucor circinelloides. Curr. Microbiol. 39: 259-64.

Sambrook, J. Fritsch, E. F. and Maniatis, T. 1989. Molecular cloning: A laboratory manual, 2^{nd} edn. Cold Spring Harbor Laboratory Press, Cold Spring Harbor, N.Y.

Schuren, F. H. J. and Wessels. J. G. H. 1994. Highly-efficient transformation of the homobasidiomycete Schizophyllum commune to phleomycin resistance. Curr. Genet. 26: 179-83.

Sock, J. and Rohringer, R. 1988. Activity staining of blotted enzymes by reaction coupling with transfer membrane-immobilized auxiliary enzymes. Anal. Biochem. 171: 310-19.

Sorol, M. R., Pastori, R. L., Muro, A., Moreno, S. and Rossi, S. 2000. Structural and functional analysis of the cAMP binding domain from the regulatory subunit of Mucor rouxii protein kinase A. Arch. Biochem. Biophys. 382: 173-81.

Strøman, P., Bruyant, P., Jepsen, L. P., Arnau, J. and Wind, A. 1990. Secretion of active calf chymosin from Mucor circinelloides. In 5^{th} European Congress on Biotechnology, Proceedings vol. 1 (C. Christiansen, L. Munck and J. Villadsen, Eds.), pp. 203-206. Munksgaard, Copenhagen.

Taylor, S. S., Buechler, J. A. and Yonemoto, W. 1990. cAMP-dependent protein kinase: framework for a diverse family of regulatory enzymes Annu. Rev. Biochem. 59: 971-1005.

Trinci, A. P. 1974. A study of the kinetics of hyphal extension and branch initiation of fungal mycelia. J Gen Microbiol. 81:225-36.

van den Hondel, C. A. M. J. J. and Punt, P. J. 1991. Gene tranfer system and vector devlopment for filamentous fungi. In Applied molecular genetics of fungi (J. F. Peberdy, C. E. Caten, J. E. Ogden and J. W. Bennett, Eds.), pp. 1-28. Cambridge University Press, Cambridge.

van den Rhee, M. D. Graa, P. M. A., Huizing, H. J. and Mooibroek, H. 1996. Transformation of the cultivated mushroom, Agaricus bisporus, to hygromycin B resistance. Mol. Gen. Genet. 250: 252-58.

van Heeswijck, R. and Roncero, M. I. G. 1984. High frequency transformation of Mucor with recombinant plasmid DNA. Carlsberg Res. Commun. 49:691-702.

van Heeswijck, R., Roncero, M. I. G. and Jepsen, L. P. 1988. Genetic analysis and manipulation of Mucor species by DNA mediated transformation. In Modern methods of plant analysis vol. VII. (J. H. Linskens, and J. F. Jackson, Eds.). pp. 207-220. Springer-Verlag, Berlin.

Verdoes, J. C. Wery, J., Boekhout, T. and Van Ooyen, A. J. 1997. Molecular characterization of the glyceraidehyde-3-phosphate dehydrogenase gene of Phaffia rhodoxyma. Yeast 13: 1231-42.

Wada, M., Beppu, T. and Horinouchi, S. 1996. Integrative transformation of the zygomycete Rhizomucor pusillus by homologous recombination. Appl Microbiol Biotechnol. 45: 652-7.

Wartmann, T., Kruger, A., Adler, K., Duc, B. M., Kunze, I. and Kunze, G. 1995. Temperature-dependent dimorphism of the yeast Arxula adeninivorans Ls3. Antonie Van Leeuwenhoek 68: 215-23.

Waterham, H. R., Digan, M. E., Koutz, P. J., Lair, S. V. and Cregg, J. M. 1997. Isolation of the Pichia pastoris glyceraldehyde-3-phosphate dehydrogenase gene and regulation and use of its promoter. Gene 186: 37-44.

Wolff, A. M. and Arnau, J. 2002. Cloning of glyceraldehyde-3-phosphate dehydrogenase-encoding genes in Mucor circinelloides (syn. racemosus) and use of the gpd1 promoter for recombinant protein production. Fungal Gen. Biol. 35: 21-9.

Wöstemeyer, J., Burmester, A. and Weigel, C. 1987. Neomycin resistance as a dominantly selectable marker for transformation of the zygomycete Absidia glauca. Curr. Genet. 12: 625-7.

Wösten, H. A., Moukha, S. M., Sietsma, J. H. and Wessels, J. G. 1991. Localization of growth and secretion of proteins in Aspergillus niger. J. Gen. Microbiol. 137: 2017-23.

Yue, C., Cavallo, L. M., Alspaugh, J. A., Wang, P., Cox, G. M., Perfect, J. R. and Heitman, J. 1999. The STE12α homolog is required for haploid filamentation but largely dispensable for mating and virulence in Cryptococcus neoformans. Genetics 153: 1601-15.

1. An isolated polynucleotide comprising
i) a first nucleotide sequence encoding at least one regulator of morphology capable of regulating the morphology of a dimorphic fungal cell, and operably linked thereto
ii) a second nucleotide sequence comprising an expression signal capable of directing the expression of the first nucleotide sequence in a dimorphic fungal cell,
wherein the first and second nucleotide sequences are not natively associated.

2. Polynucleotide according to claim 1, wherein the dimorphic fungal cell is capable of growing as a multinucleated cell having a unicellular, essentially spherical morphology and/or capable of growing as a mycelium having a filamentous structure and comprising multinucleated cells.

3. Polynucleotide according to claim 2, wherein the multinucleated cells are multipolar.

4. Polynucleotide according to claim 2, wherein the dimorphic fungal cell belongs to the class of Zygomycetes.

5. Polynucleotide according to claim 4, wherein the dimorphic fungal cell belongs to the order of Mucorales.

6. Polynucleotide according to claim 4, wherein the dimorphic fungal cell belongs to a genus selected from the group of genera consisting of Mucor, Thermomucor, Rhizomucor, Mycotypha, Rhizopus, and Cokeromyces.

7. Polynucleotide according to claim 4, wherein the dimorphic fungal cell belongs to the genus Mucor.

8. Polynucleotide according to claim 4, wherein the dimorphic fungal cell is a Mucor species selected from the group of Mucor species consisting of M. circinelloides, M. hiemalis, M. rouxii, M. genevensis, M. bacilliformis, and M. subtillissimus.

9. Polynucleotide according to claim 8, wherein the Mucor species is M. circinelloides.

10. Polynucleotide according to claim 1, wherein the dimorphic fungal cell is capable of growing as a uninucleated cell having a unicellular, essentially spherical morphology, and/or capable of growing as a filamentous structure comprising uninucleated cells.

11. Polynucleotide according to claim 10, wherein the dimorphic fungal cell is selected from the group consisting of Yarrowia, Candida and Arxula.

12. Polynucleotide according to claim 1, wherein the first nucleotide sequence is selected from the group consisting of
i) a polynucleotide comprising nucleotides 542 to 1930 of SEQ ID NO:1, and
ii) a polynucleotide comprising or essentially consisting of the coding sequence of pkaR encoding the regulatory subunit of protein kinase A (PKAR) of Mucor circinelloides, as deposited with DSMZ under accession number DSM 14062; and
iii) a polynucleotide encoding a polypeptide having the amino acid sequence as shown in SEQ ID NO:2; and
iv) a polynucleotide encoding a fragment of a polypeptide encoded by polynucleotides (i), (ii) or (iii), wherein said fragment
a) has Mucor circinelloides protein kinase A regulatory subunit acitivty and is a regulator of morphology of a dimorphic fungal cell; and/or
b) is recognised by an antibody, or a binding fragment thereof, which is capable of recognising a cAMP binding domain of Mucor circinelloides protein kinase A, wherein said cAMP binding domain is comprised by the polypeptide having the amino acid sequence as shown in SEQ ID NO:2; and/or
c) is competing with a polypeptide comprising or essentially consisting of the amino acid sequence as shown in SEQ ID NO:2 for binding to at least one predetermined binding partner, including cAMP and/or the catalytic subunit for protein kinase A; and
v) a polynucleotide, the complementary strand of which hybridizes, under stringent conditions, with a polynucleotide as defined in any of (i), (ii) (iii), and (iv), and encodes a polypeptide that
a) has Mucor circinelloides protein kinase A regulatory subunit acitivty and is a regulator of morphology of a dimorphic fungal cell; and/or
b) is recognised by an antibody, or a binding fragment thereof, which is capable of recognising a cAMP binding domain of Mucor circinelloides protein kinase A, wherein said cAMP binding domain is comprised by the polypeptide having the amino acid sequence as shown in SEQ ID NO:2; and/or
c) is competing with a polypeptide comprising or essentially consisting of the amino acid sequence as shown in SEQ ID NO:2 for binding to at least one predetermined binding partner, including cAMP and/or the catalytic subunit for protein kinase A; and
vi) a polynucleotide comprising a nucleotide sequence which is degenerate to the nucleotide sequence of a polynucleotide as defined in any of (iv) and (v),
and the complementary strand of such a polynucleotide.

13. Polynucleotide according to claim 1, wherein the first nucleotide sequence comprises nucleotides 542 to 1930 of SEQ ID NO:1.

14. Polynucleotide according to claim 1, wherein the first nucleotide sequence comprises or essentially consists of the coding sequence of pkaR encoding the regulatory subunit of protein kinase A (PKAR) of Mucor circinelloides, as deposited with DSMZ under accession number DSM 14062.

15. Polynucleotide according to claim 1, wherein the first nucleotide sequence encodes a polypeptide having the amino acid sequence as shown in SEQ ID NO:2.

16. Polynucleotide according to claim 1, wherein the first nucleotide sequence encodes a fragment of the polypeptide having the amino acid sequence as shown in SEQ ID NO:2, wherein said fragment
a) has Mucor circinelloides protein kinase A regulatory subunit acitivty and is a regulator of morphology of a dimorphic fungal cell; and/or
b) is recognised by an antibody, or a binding fragment thereof, which is capable of recognising a cAMP binding domain of Mucor circinelloides protein kinase A, wherein said cAMP binding domain is comprised by the polypeptide having the amino acid sequence as shown in SEQ ID NO:2; and/or
c) is competing with a polypeptide comprising or essentially consisting of the amino acid sequence as shown in SEQ ID NO:2 for binding to at least one predetermined binding partner, including cAMP and/or the catalytic subunit for protein kinase A.

17. Polynucleotide according to claim 1, wherein the complementary strand of said polynucleotide hybridizes under stringent conditions with the polynucleotide according to any of claims 13 to 16 and encodes a polypeptide that
a) has Mucor circinelloides protein kinase A regulatory subunit acitivty and is a regulator of morphology of a dimorphic fungal cell; and/or
b) is recognised by an antibody, or a binding fragment thereof, which is capable of recognising a cAMP binding domain of Mucor circinelloides protein kinase A, wherein said cAMP binding domain is comprised by the polypeptide having the amino acid sequence as shown in SEQ ID NO:2; and/or
c) is competing with a polypeptide comprising or essentially consisting of the amino acid sequence as shown in SEQ ID NO:2 for binding to at least one predetermined binding partner, including cAMP and/or the catalytic subunit for protein kinase A.

18. Polynucleotide according to claim 1 and comprising a nucleotide sequence which is degenerate to the first nucleotide sequence according to any of claims 16 and 17.

19. Polynucleotide according to claim 12, said polynucleotide comprising the complementary strand of the polynucleotide according to any of claims 12 to 18.

20. Polynucleotide according to claim 1, wherein the first nucleotide sequence is selected from the group consisting of
i) a polynucleotide comprising nucleotides 534 to 2471 of SEQ ID NO:11, and
ii) a polynucleotide comprising or essentially consisting of the coding sequence of pkaC encoding a catalytic subunit of protein kinase A (PKAC) of Mucor circinelloides, as deposited with DSMZ under accession number DSM 14839; and
iii) a polynucleotide encoding a polypeptide having the amino acid sequence as shown in SEQ ID NO:12; and
iv) a polynucleotide encoding a fragment of a polypeptide encoded by polynucleotides (i), (ii) or (iii), wherein said fragment
a) has Mucor circinelloides catalytic subunit of protein kinase A activity and is a regulator of morphology of a dimorphic fungal cell; and/or
b) is recognised by an antibody, or a binding fragment thereof, which is capable of recognising a protein kinase A binding domain of Mucor circinelloides PKAC, wherein said domain is comprised by the polypeptide having the amino acid sequence as shown in SEQ ID NO:12; and/or
c) is competing with a polypeptide comprising or essentially consisting of the amino acid sequence as shown in SEQ ID NO:12 for binding to at least one predetermined binding partner, including PKAR; and
v) a polynucleotide, the complementary strand of which hybridizes, under stringent conditions, with a polynucleotide as defined in any of (i), (ii) (iii), and (iv), and encodes a polypeptide that
a) has Mucor circinelloides catalytic subunit of protein kinase A activity and is a regulator of morphology of a dimorphic fungal cell; and/or
b) is recognised by an antibody, or a binding fragment thereof, which is capable of recognising a protein kinase A binding domain of Mucor circinelloides PKAC, wherein said domain is comprised by the polypeptide having the amino acid sequence as shown in SEQ ID NO:12; and/or
c) is competing with a polypeptide comprising or essentially consisting of the amino acid sequence as shown in SEQ ID NO:12 for binding to at least one predetermined binding partner, including PKAR; and
vi) a polynucleotide comprising a nucleotide sequence which is degenerate to the nucleotide sequence of a polynucleotide as defined in any of (iv) and (v),
and the complementary strand of such a polynucleotide.

21. Polynucleotide according to claim 1, wherein the first nucleotide sequence comprises nucleotides 534 to 2471 of SEQ ID NO:11.

22. Polynucleotide according to claim 1, wherein the first nucleotide sequence comprises or essentially consists of the coding sequence of pkaC encoding a catalytic subunit of protein kinase A (PKAC) of Mucor circinelloides, as deposited with DSMZ under accession number DSM 14839.

23. Polynucleotide according to claim 1, wherein the first nucleotide sequence encodes a polypeptide having the amino acid sequence as shown in SEQ ID NO:12.

24. Polynucleotide according to claim 1, wherein the first nucleotide sequence encodes a fragment of the polypeptide having the amino acid sequence as shown in SEQ ID NO:12, wherein said fragment
a) has Mucor circinelloides catalytic subunit of protein kinase A activity and is a regulator of morphology of a dimorphic fungal cell; and/or
b) is recognised by an antibody, or a binding fragment thereof, which is capable of recognising a protein kinase A binding domain of Mucor circinelloides PKAC, wherein said domain is comprised by the polypeptide having the amino acid sequence as shown in SEQ ID NO:12; and/or
c) is competing with a polypeptide comprising or essentially consisting of the amino acid sequence as shown in SEQ ID NO:12 for binding to at least one predetermined binding partner, including PKAR.

25. Polynucleotide according to claim 1, wherein the complementary strand of said polynucleotide hybridizes under stringent conditions with the polynucleotide according to any of claims 21 to 24 and encodes a polypeptide that
a) has Mucor circinelloides catalytic subunit of protein kinase A activity and is a regulator of morphology of a dimorphic fungal cell; and/or
b) is recognised by an antibody, or a binding fragment thereof, which is capable of recognising a protein kinase A binding domain of Mucor circinelloides PKAC, wherein said domain is comprised by the polypeptide having the amino acid sequence as shown in SEQ ID NO:12; and/or
c) is competing with a polypeptide comprising or essentially consisting of the amino acid sequence as shown in SEQ ID NO:12 for binding to at least one predetermined binding partner, including PKAR.

26. Polynucleotide according to claim 1 and comprising a nucleotide sequence which is degenerate to the first nucleotide sequence according to any of claims 24 and 25.

27. Polynucleotide according to claim 20, said polynucleotide comprising the complementary strand of the polynucleotide according to any of claims 21 to 26.

28. Polynucleotide according to claim 1, wherein said first and/or second nucleotide sequence is derived from a microbial cell.

29. Polynucleotide according to claim 28, wherein said microbial cell is selected from the group of microbial cells consisting of eukaryotic microbial cells and procaryotic cells.

30. Polynucleotide according to claim 29, wherein said microbial cell is a eukaryotic microbial cell.

31. Polynucleotide according to claim 30, wherein said eukaryotic microbial cell is selected from the group of eukaryotic cells consisting of fungal cells and a yeast cells.

32. Polynucleotide according to claim 31, wherein said eukaryotic microbial cell is a fungal cell, including a filamentous fungal cell.

33. Polynucleotide according to claim 32, wherein said fungal cell is a dimorphic fungal cell.

34. Polynucleotide according to claim 32, wherein said fungal cell belongs to the class of Zygomycetes.

35. Polynucleotide according to claim 32, wherein said fungal cell belongs to the order of Mucorales.

36. Polynucleotide according to claim 32, wherein said fungal cell belongs to the genus selected from the group of genera consisting of Mucor, Mycotypha, and Cokeromyces.

37. Polynucleotide according to claim 32, wherein said fungal cell belongs to the genus Mucor.

38. Polynucleotide according to claim 37, wherein said fungal cell is a Mucor species selected from the group of Mucor species consisting of M. circinelloides; M. rouxii, M. genevensis, M. bacilliformis, and M. subtillissimus.

39. Polynucleotide according to claim 37, wherein the Mucor species is M. circinelloides.

40. Polynucleotide according to claim 1, wherein the second nucleotide sequence comprising an expression signal comprises at least one element of a promoter region capable of being regulated, including being induced or repressed, during growth of the dimorphic fungal cell, by any one or more factors including
a) the composition of the growth medium, including at least one of carbon source, nitrogen source including amino acids or precursors thereof, oxygen content, ionic strength, including NaCl content, pH, low molecular weight compounds, cAMP, and the presence or absence of a cell constituent, or a precursor thereof,
b) the temperature of the growth medium, including any change thereof, including an upshift eliciting the expression of one or more heat shock genes,
c) the growth phase of the dimorphic fungal cell, and
d) the growth rate of the dimorphic fungal cell.

41. Polynucleotide according to claim 40, wherein the induction, derepression or repression of the expression of the first nucleotide sequence being operably linked to the expression signal is an induction or a repression of said expression, as compared to a predetermined expression level, by at least a factor of 2.0.

42. Polynucleotide according to claim 40, wherein the at least one element of the promoter region comprised by the expression signal is regulated, during growth of the dimorphic fungal cell, by the carbon source of the growth medium.

43. Polynucleotide according to claim 40, wherein the at least one element of the promoter region comprised by the expression signal is regulated, during growth of the dimorphic fungal cell, by the oxygen content and the carbon source of the growth medium.

44. Polynucleotide according to claim 40, wherein the expression of the first nucleotide sequence being operably linked to the expression signal comprising the at least one element of the promoter region is induced by the presence in the growth medium, or the addition to the growth medium, of a carbon source.

45. Polynucleotide according to claim 44, wherein the carbon source comprises a hexose

46. Polynucleotide according to claim 45, wherein the carbon source is selected from glucose and galactose.

47. Polynucleotide according to claim 40, wherein the at least one element of the promoter region comprised by the expression signal is selected from the group consisting of
i) a polynucleotide comprising nucleotides 1 to 741 of SEQ ID NO:9,
ii) a polynucleotide comprising or essentially consisting of the promoter region of gpd1 of Mucor circinelloides, as deposited with DSMZ under accession number DSM 14066; and
iii) a polynucleotide comprising at least one fragment of SEQ ID NO:9, wherein said fragment
a) is capable of directing gene expression in a dimorphic fungal cell; and/or
b) is regulatable, during growth of the dimorphic fungal cell, by at least one factor capable of regulating gene expression directed by SEQ ID NO:9; and
iv) a polynucleotide, the complementary strand of which hybridizes, under stringent conditions, with a polynucleotide as defined in any of (i), (ii) and (iii), wherein said polynucleotide
a) is capable of directing gene expression in a dimorphic fungal cell; and/or
b) is regulatable, during growth of the dimorphic fungal cell, by at least one factor capable of regulating gene expression directed by SEQ ID NO:9,
and the complementary strand of such a polynucleotide.

48. Polynucleotide according to claim 40, wherein the at least one element of the promoter region comprised by the expression signal comprises nucleotides 1 to 741 of SEQ ID NO:9, or a fragment thereof, wherein said fragment is capable of directing gene expression in a dimorphic fungal cell and is regulatable, during growth of the dimorphic fungal cell, by at least one factor capable of regulating gene expression directed by SEQ ID NO:9.

49. Polynucleotide according to claim 48, wherein said factor is selected from the group consisting of
a) the composition of the growth medium, including at least one of carbon source, nitrogen source including amino acids or precursors thereof, oxygen content, ionic strength, including NaCl content, pH, low molecular weight compounds, cAMP, and the presence or absence of a cell constituent, or a precursor thereof
b) the temperature of the growth medium, including any change thereof, including an upshift eliciting the expression of one or more heat shock genes,
c) the growth phase of the dimorphic fungal cell, and
d) the growth rate of the dimorphic fungal cell.

50. Polynucleotide according to claim 40, wherein the at least one element of the promoter region comprised by the expression signal comprises nucleotides 1 to 741 of SEQ ID NO: 9, or the promoter region of gpd1 of M. circinelloides, as deposited with DSMZ under accession number DSM 14066.

51. Polynucleotide according to claim 40, wherein at least one element of the promoter region comprised by the expression signal is selected from the group consisting of
i) a polynucleotide comprising nucleotides 1 to 755 of SEQ ID NO:10,
ii) a polynucleotide comprising or essentially consisting of the promoter region of prnC of Mucor circinelloides, as deposited with DSMZ under accession number DSM 14067; and
iii) a polynucleotide comprising at least one fragment of SEQ ID NO:10, wherein said fragment
a) is capable of directing gene expression in a dimorphic fungal cell; and/or
b) is regulatable, during growth of the dimorphic fungal cell, by at least one factor capable of regulating gene expression directed by nucleotides 1 to 755 of SEQ ID NO:10; and
iv) a polynucleotide, the complementary strand of which hybridizes, under stringent conditions, with a polynucleotide as defined in any of (i), (ii) and (iii), wherein said polynucleotide
a) is capable of directing gene expression in a dimorphic fungal cell; and/or
b) is regulatable, during growth of the dimorphic fungal cell, by at least one factor capable of regulating gene expression directed by nucleotides 1 to 755 of SEQ ID NO:10,
and the complementary strand of such a polynucleotide.

52. Polynucleotide according to claim 40, wherein the at least one element of the promoter region comprised by the expression signal comprises nucleotides 1 to 755 of SEQ ID NO:10, or a fragment thereof, wherein said fragment is capable of directing gene expression in a dimorphic fungal cell and is regulatable, during growth of the dimorphic fungal cell, by at least one factor capable of regulating gene expression directed by nucleotides 1 to 755 of SEQ ID NO:10.

53. Polynucleotide according to claim 52, wherein said factor is selected from the group consisting of
a) the composition of the growth medium, including at least one of carbon source, nitrogen source including amino acids or precursors thereof, oxygen content, ionic strength, including NaCl content, pH, low molecular weight compounds, cAMP, and the presence or absence of a cell constituent, or a precursor thereof
b) the temperature of the growth medium, including any change thereof, including an upshift eliciting the expression of one or more heat shock genes,
c) the growth phase of the dimorphic fungal cell, and
d) the growth rate of the dimorphic fungal cell.

54. Polynucleotide according to claim 40, wherein the at least one element of the promoter region comprised by the expression signal comprises nucleotides 1 to 755 of SEQ ID NO: 10, or the promoter region of prnC of M. circinelloides, as deposited with DSMZ under accession number DSM 14067.

55. Polynucleotide according to claim 40, wherein the at least one element of the promoter region comprised by the expression signal is selected from the group consisting of
i) a polynucleotide comprising nucleotides 1 to 927 of SEQ ID NO:13,
ii) a polynucleotide comprising or essentially consisting of the promoter region of tubA of Mucor circinelloides, as deposited with DSMZ under accession number DSM 14841; and
iii) a polynucleotide comprising at least one fragment of SEQ ID NO:13, wherein said fragment
a) is capable of directing gene expression in a dimorphic fungal cell; and/or
b) is regulatable, during growth of the dimorphic fungal cell, by at least one factor capable of regulating gene expression directed by SEQ ID NO:13; and
iv) a polynucleotide, the complementary strand of which hybridizes, under stringent conditions, with a polynucleotide as defined in any of (i), (ii) and (iii), wherein said polynucleotide
a) is capable of directing gene expression in a dimorphic fungal cell; and/or
b) is regulatable, during growth of the dimorphic fungal cell, by at least one factor capable of regulating gene expression directed by SEQ ID NO:13,
and the complementary strand of such a polynucleotide.

56. Polynucleotide according to claim 40, wherein the at least one element of the promoter region comprised by the expression signal comprises nucleotides 1 to 927 of SEQ ID NO:13, or a fragment thereof, wherein said fragment is capable of directing gene expression in a dimorphic fungal cell and is regulatable, during growth of the dimorphic fungal cell, by at least one factor capable of regulating gene expression directed by SEQ ID NO:13.

57. Polynucleotide according to claim 56, wherein said factor is selected from the group consisting of
a) the composition of the growth medium, including at least one of carbon source, nitrogen source including amino acids or precursors thereof, oxygen content, ionic strength, including NaCl content, pH, low molecular weight compounds, cAMP, and the presence or absence of a cell constituent, or a precursor thereof
b) the temperature of the growth medium, including any change thereof, including an upshift eliciting the expression of one or more heat shock genes,
c) the growth phase of the dimorphic fungal cell, and
d) the growth rate of the dimorphic fungal cell.

58. Polynucleotide according to claim 40, wherein the at least one element of the promoter region comprised by the expression signal comprises nucleotides 1 to 927 of SEQ ID NO: 13, or the promoter region of tubA of M. circinelloides, as deposited with DSMZ under accession number DSM 14841.

59. Polynucleotide according to claim 40, wherein the at least one element of the promoter region comprised by the expression signal is selected from the group consisting of
i) a polynucleotide comprising nucleotides 1 to 419 of SEQ ID NO:14,
ii) a polynucleotide comprising or essentially consisting of the promoter region of gal1 of Mucor circinelloides, as deposited with EMBL under accession number AJ438267; and
iii) a polynucleotide comprising at least one fragment of SEQ ID NO:14, wherein said fragment
a) is capable of directing gene expression in a dimorphic fungal cell; and/or
b) is regulatable, during growth of the dimorphic fungal cell, by at least one factor capable of regulating gene expression directed by SEQ ID NO:14; and
iv) a polynucleotide, the complementary strand of which hybridizes, under stringent conditions, with a polynucleotide as defined in any of (i), (ii) and (iii), wherein said polynucleotide
a) is capable of directing gene expression in a dimorphic fungal cell; and/or
b) is regulatable, during growth of the dimorphic fungal cell, by at least one factor capable of regulating gene expression directed by SEQ ID NO:14,
and the complementary strand of such a polynucleotide.

60. Polynucleotide according to claim 40, wherein the at least one element of the promoter region comprised by the expression signal comprises nucleotides 1 to 419 of SEQ ID NO:14, or a fragment thereof, wherein said fragment is capable of directing gene expression in a dimorphic fungal cell and is regulatable, during growth of the dimorphic fungal cell, by at least one factor capable of regulating gene expression directed by SEQ ID NO:14.

61. Polynucleotide according to claim 60, wherein said factor is selected from the group consisting of
i) the composition of the growth medium, including at least one of carbon source, nitrogen source including amino acids or precursors thereof, oxygen content, ionic strength, including NaCl content, pH, low molecular weight compounds, cAMP, and the presence or absence of a cell constituent, or a precursor thereof
ii) the temperature of the growth medium, including any change thereof, including an upshift eliciting the expression of one or more heat shock genes,
iii) the growth phase of the dimorphic fungal cell, and
iv) the growth rate of the dimorphic fungal cell.

62. Polynucleotide according to claim 40, wherein the at least one element of the promoter region comprised by the expression signal comprises nucleotides 1 to 419 of SEQ ID NO: 14, or the promoter region of gal1 of M. circinelloides, as deposited with EMBL under accession number AJ438267.

63. Polynucleotide according to claim 1 and operably linked to a further polynucleotide selected from the group of polynucleotides consisting of a 3′ untranslated region, or a fragment thereof, and/or a 5′ upstream region, or a fragment thereof.

64. Polynucleotide according to claim 1, wherein the at least one regulator of morphology is a polypeptide capable of regulating gene transcription in a dimorphic fungal cell by forming an interaction with a recognition motif of a promoter region having an affinity for the at least one regulator of morphology.

65. Polynucleotide according to claim 1, wherein the expression of said first polynucleotide results in the production of the at least one regulator in an increased or decreased amount, as compared to the amount of regulator produced, when the first polynucleotide encoding the at least one regulator is operably linked to the native promoter region under substantially identical growth conditions, and wherein the expression of said first polynucleotide and the production of the at least one regulator in an increased or a decreased amount results in an improved filamentation, or in a dimorphic shift of the dimorphic fungal cell.

66. Polynucleotide according to claim 65, wherein an increased amount of the at least one regulator of morphology is produced from the expression of the first nucleotide sequence encoding said regulator.

67. Polynucleotide according to claim 65, wherein a decreased amount of the at least one regulator of morphology is produced from the expression of the first nucleotide sequence encoding said regulator.

68. Polynucleotide according to claim 65, wherein the amount of regulator produced is increased or decreased at least by a factor of 2.0.

69. Polynucleotide according to claim 65, wherein the at least one regulator of morphology is a polypeptide encoded by a first polynucleotide comprising a first polynucleotide sequence forming part of a cAMP-dependent signal transduction pathway of a microbial cell, including a fungal cell, including a Mucor species, including M. circinelloides,
wherein the expression of said first polynucleotide results in the production of the at least one regulator in an increased or decreased amount, as compared to the amount of regulator produced, when the first polynucleotide encoding the at least one regulator is operably linked to the native promoter region under substantially identical growth conditions,
wherein the expression of said first polynucleotide and the production of the at least one regulator in an increased or a decreased amount results in an improved filamentation, or in a dimorphic shift of the dimorphic fungal cell.

70. Polynucleotide according to claim 65, wherein the at least one regulator of morphology is a polypeptide encoded by a first polynucleotide comprising a first polynucleotide sequence forming part of a MAP kinase-dependent signal transduction pathway of a microbial cell, including a fungal cell, including a Mucor species, including M. circinelloides,
wherein the expression of said first polynucleotide results in the production of the at least one regulator in an increased or decreased amount, as compared to the amount of regulator produced, when the first polynucleotide encoding the at least one regulator is operably linked to the native promoter region under substantially identical growth conditions,
wherein the expression of said first polynucleotide and the production of the at least one regulator in an increased or a decreased amount results in an improved filamentation, or in a dimorphic shift of the dimorphic fungal cell.

71. Polynucleotide according to claim 65, wherein the dimorphic shift of the dimorphic fungal cell is a shift from a first morphological condition of the dimorphic fungal cell characterised by a unicellular, essentially spherical morphology, to a second morphological condition of the dimorphic fungal cell, wherein the fungal cell comprises a mycelium and is characterised by filamentous growth.

72. Polynucleotide according to claim 65, wherein the dimorphic shift of the dimorphic fungal cell is a shift from a second morphological condition of the dimorphic fungal cell, wherein the fungal cell comprises a mycelium and is characterised by filamentous growth, to a first morphological condition of the dimorphic fungal cell characterised by a unicellular, essentially spherical morphology.

73. Polynucleotide according to any of claims 71 and 72, wherein the first morphological condition of the fungal cell characterised by a unicellular, essentially spherical morphology is further characterised by an essentially isodiametrical or spherical shape of the fungal cells.

74. Polynucleotide according to any of claims 71 and 72, wherein the second morphological condition of the dimorphic fungal cell, wherein the fungal cell comprises a mycelium and is characterised by filamentous growth, is further characterised by an essentially elongated, hyphal cell shape resulting from a polarised growth of a fungal cell characterised by the first morphological condition.

75. An isolated polypeptide capable of regulating the morphology of a dimorphic fungal cell and encoded by the first nucleotide sequence of claim 1.

76. An isolated polypeptide according to claim 75 comprising or essentially consisting of the amino acid sequence of SEQ ID NO:2, or a fragment thereof, or a polypeptide functionally equivalent to SEQ ID NO: 2, or a fragment thereof, wherein said fragment or functionally equivalent polypeptide
a) has Mucor circinelloides protein kinase A regulatory subunit acitivty and is a regulator of morphology of a dimorphic fungal cell; and/or
b) is recognised by an antibody, or a binding fragment thereof, which is capable of recognising a cAMP binding domain of Mucor circinelloides protein kinase A, wherein said cAMP binding domain is comprised by the polypeptide having the amino acid sequence as shown in SEQ ID NO:2; and/or
c) is competing with a polypeptide comprising or essentially consisting of the amino acid sequence as shown in SEQ ID NO:2 for binding to at least one predetermined binding partner, including cAMP and/or PKAC.

77. An isolated polypeptide according to claim 75 comprising or essentially consisting of the amino acid sequence of SEQ ID NO:12, or a fragment thereof, or a polypeptide functionally equivalent to SEQ ID NO: 12, or a fragment thereof, wherein said fragment or functionally equivalent polypeptide
a) has Mucor circinelloides catalytic subunit of protein kinase A activity and is a regulator of morphology of a dimorphic fungal cell; and/or
b) is recognised by an antibody, or a binding fragment thereof, which is capable of recognising a protein kinase A binding domain of Mucor circinelloides PKAC, wherein said domain is comprised by the polypeptide having the amino acid sequence as shown in SEQ ID NO:12; and/or
c) is competing with a polypeptide comprising or essentially consisting of the amino acid sequence as shown in SEQ ID NO:12 for binding to at least one predetermined binding partner, including PKAR.

78. Polypeptide according to claim 75, wherein a substantially identical morphological shift is obtained from the production in a dimorphic fungal cell, under substantially identical conditions, of substantially identically amounts of i) the polypeptide comprising the at least one regulator of morphology of a dimorphic fungal cell, and ii) a functionally equivalent polypeptide comprising a functionally equivalent regulator of morphology, including any fragments thereof.

79. Polypeptide according to claim 78, wherein the functionally equivalent polypeptide, or a fragment thereof, comprises at least one conservative amino acid substitution.

80. A extrachromosomal, recombinant DNA molecule, preferably in the form of an expression vector, comprising the polynucleotide according to any of claims 1 to 74.

81. Recombinant DNA molecule according to claim 80 and further comprising a selectable marker.

82. A fungal host cell transfected or transformed with the polynucleotide according to any of claims 1 to 74, or the vector according to any of claims 80 and 81.

83. Fungal host cell according to claim 82, wherein said host organism is a dimorphic fungal cell.

84. Fungal cell according to claim 82 or dimorphic fungal cell according to claim 83, wherein said fungal cell or said dimorphic cell further comprises
i) at least one nucleotide sequence encoding a gene product, and operably linked thereto, and
ii) at least one further nucleotide sequence comprising a further expression signal capable of directing the expression in a dimorphic fungal cell of the at least one nucleotide sequence encoding the gene product, wherein said further expression signal is regulatable, during growth of the dimorphic fungal cell, by one or more of
a) the composition of the growth medium, including at least one of carbon source, nitrogen source including amino acids or precursors thereof, oxygen content, ionic strength, including NaCl content, pH, low molecular weight compounds, cAMP, and the presence or absence of a cell constituent, or a precursor thereof,
b) the temperature of the growth medium, including any change thereof, including an upshift eliciting the expression of one or more heat shock genes,
c) the growth phase of the dimorphic fungal cell, and
d) the growth rate of the dimorphic fungal cell.
wherein the nucleotide sequence encoding the gene product and the further nucleotide sequence comprising the regulatable expression signal are not natively associated.

85. Dimorphic fungal cell comprising
i) at least one nucleotide sequence encoding a gene product, and operably linked thereto, and
ii) at least one further nucleotide sequence comprising a further expression signal capable of directing the expression in a dimorphic fungal cell of the at least one nucleotide sequence encoding the gene product, wherein said further expression signal is regulatable, during growth of the dimorphic fungal cell, by one or more of
a) the composition of the growth medium, including at least one of carbon source, nitrogen source including amino acids or precursors thereof, oxygen content, ionic strength, including NaCl content, pH, low molecular weight compounds, cAMP, and the presence or absence of a cell constituent, or a precursor thereof,
b) the temperature of the growth medium, including any change thereof, including an upshift eliciting the expression of one or more heat shock genes,
c) the growth phase of the dimorphic fungal cell, and
d) the growth rate of the dimorphic fungal cell,
wherein the nucleotide sequence encoding the gene product and the further nucleotide sequence comprising the regulatable expression signal are not natively associated.

86. Dimorphic fungal cell according to claim 85 transfected or transformed with the polynucleotide according to any of claims 1 to 74, or the vector according to any of claims 80 and 81.

87. Dimorphic fungal cell according to claim 83, wherein the fungal cell belongs to the class of Zygomycetes.

88. Dimorphic fungal cell according to claim 87, wherein the fungal cell belongs to the order of Mucorales.

89. Dimorphic fungal cell according to claim 87, wherein the fungal cell belongs to a genus selected from the group of genera consisting of Mucor, Thermomucor, Rhizomucor, Mycotypha, Rhizopus, and Cokeromyces.

90. Dimorphic fungal cell according to claim 89, wherein the fungal cell belongs to the genus Mucor.

91. Dimorphic fungal cell according to claim 90, wherein the fungal cell belongs to a Mucor species selected from the group of Mucor species consisting of M. circinelloides; M. hiemalis, M. rouxii, M. genevensis, M. bacilliformis, and M. subtillissimus.

92. Dimorphic fungal cell according to claim 91, wherein the Mucor species is M. circinelloides.

93. Dimorphic fungal cell according to claim 85, wherein the fungal cell belongs to the class of Zygomycetes.

94. Dimorphic fungal cell according to claim 93, wherein the fungal cell belongs to the order of Mucorales.

95. Dimorphic fungal cell according to claim 93, wherein the fungal cell belongs to a genus selected from the group of genera consisting of Mucor, Thermomucor, Rhizomucor, Mycotypha, Rhizopus, and Cokeromyces.

96. Dimorphic fungal cell according to claim 95, wherein the fungal cell belongs to the genus Mucor.

97. Dimorphic fungal cell according to claim 96, wherein the fungal cell belongs to a Mucor species selected from the group of Mucor species consisting of M. circinelloides; M. hiemalis, M. rouxii, M. genevensis, M. bacilliformis, and M. subtillissimus.

98. Dimorphic fungal cell according to claim 97, wherein the Mucor species is M. circinelloides.

99. Dimorphic fungal cell according to claim 85, wherein the at least one further nucleotide sequence comprising the further expression signal is selected from the group consisting of
i) a polynucleotide comprising nucleotides 1 to 741 of SEQ ID NO:9,
ii) a polynucleotide comprising or essentially consisting of the promoter region of gpd1 of Mucor circinelloides, as deposited with DSMZ under accession number DSM 14066; and
iii) a polynucleotide comprising at least one fragment of SEQ ID NO:9, wherein said fragment
a) is capable of directing gene expression in a dimorphic fungal cell; and/or
b) is regulatable, during growth of the dimorphic fungal cell, by at least one factor capable of regulating gene expression directed by SEQ ID NO:9; and
iv) a polynucleotide, the complementary strand of which hybridizes, under stringent conditions, with a polynucleotide as defined in any of (i), (ii) and (iii), wherein said polynucleotide
a) is capable of directing gene expression in a dimorphic fungal cell; and/or
b) is regulatable, during growth of the dimorphic fungal cell, by at least one factor capable of regulating gene expression directed by SEQ ID NO:9,
and the complementary strand of such a polynucleotide.

100. Dimorphic fungal cell according to claim 85, wherein the at least one further nucleotide sequence comprising the further expression signal is selected from the group consisting of
i) a polynucleotide comprising nucleotides 1 to 755 of SEQ ID NO:10,
ii) a polynucleotide comprising or essentially consisting of the promoter region of prnC of Mucor circinelloides, as deposited with DSMZ under accession number DSM 14067; and
iii) a polynucleotide comprising at least one fragment of SEQ ID NO:10, wherein said fragment
a) is capable of directing gene expression in a dimorphic fungal cell; and/or
b) is regulatable, during growth of the dimorphic fungal cell, by at least one factor capable of regulating gene expression directed by SEQ ID NO:10; and
iv) a polynucleotide, the complementary strand of which hybridizes, under stringent conditions, with a polynucleotide as defined in any of (i), (ii) and (iii), wherein said polynucleotide
a) is capable of directing gene expression in a dimorphic fungal cell; and/or
b) is regulatable, during growth of the dimorphic fungal cell, by at least one factor capable of regulating gene expression directed by SEQ ID NO:10,
and the complementary strand of such a polynucleotide.

101. Dimorphic fungal cell according to claim 85, wherein the at least one further nucleotide sequence comprising the further expression signal is selected from the group consisting of
i) a polynucleotide comprising nucleotides 1 to 927 of SEQ ID NO:13
ii) a polynucleotide comprising or essentially consisting of the promoter region of the tubA gene of Mucor circinelloides, as deposited with DSMZ under accession number DSM 14841; and
iii) a polynucleotide comprising at least one fragment of SEQ ID NO:13, wherein said fragment
a) is capable of directing gene expression in a dimorphic fungal cell; and/or
b) is regulatable, during-growth of the dimorphic fungal cell, by at least one factor capable of regulating gene expression directed by SEQ ID NO:13; and
iii) a polynucleotide, the complementary strand of which hybridizes, under stringent conditions, with a polynucleotide as defined in any of (i), (ii) and (iii), wherein said polynucleotide
a) is capable of directing gene expression in a dimorphic fungal cell; and/or
b) is regulatable, during growth of the dimorphic fungal cell, by at least one factor capable of regulating gene expression directed by SEQ ID NO:13,
and the complementary strand of such a polynucleotide.

102. Dimorphic fungal cell according to claim 85, wherein the at least one further nucleotide sequence comprising the further expression signal is selected from the group consisting of
i) a polynucleotide comprising nucleotides 1 to 419 of SEQ ID NO:14,
ii) a polynucleotide comprising or essentially consisting of the promoter region of gal1 of Mucor circinelloides, as deposited with EMBL under accession number AJ438267, and
iii) a polynucleotide comprising at least one fragment of SEQ ID NO:14, wherein said fragment
a) is capable of directing gene expression in a dimorphic fungal cell; and/or
c) is regulatable, during growth of the dimorphic fungal cell, by at least one factor capable of regulating gene expression directed by SEQ ID NO:14; and
iii) a polynucleotide, the complementary strand of which hybridizes, under stringent conditions, with a polynucleotide as defined in any of (i), (ii) and (iii), wherein said polynucleotide
a) is capable of directing gene expression in a dimorphic fungal cell; and/or
b) is regulatable, during growth of the dimorphic fungal cell, by at least one factor capable of regulating gene expression directed by SEQ ID NO:14,
and the complementary strand of such a polynucleotide.

103. Dimorphic fungal cell according to claim 85, wherein the expression in the dimorphic fungal cell of the nucleotide sequence encoding the gene product results in the production of an increased amount of the gene product as compared to the production of the gene product in an at least substantially identical dimorphic fungal cell under substantially identical conditions, when the nucleotide sequence encoding the gene product is operably linked to the expression signal natively associated therewith.

104. Dimorphic fungal cell according to claim 103, wherein the amount of the gene product is increased by a factor of at least 1.25.

105. Dimorphic fungal cell according to claim 103, wherein the nucleotide sequence encoding a gene product and/or the further nucleotide sequence comprising an expression signal is derived from a fungal cell.

106. Dimorphic fungal cell according to claim 103, wherein the gene product is secreted into the growth medium.

107. Dimorphic fungal cell according to claim 103, wherein the gene product is selected from the group of gene products consisting of catalase, laccase, phenoloxidase, oxidase, oxidoreductases, cellulase, xylanase, peroxidase, lipase, hydrolase, esterase, cutinase, protease and other proteolytic enzymes, aminopeptidase, carboxypeptidase, phytase, lyase, pectinase and other pectinolytic enzymes, amylase, glucoamylase, alpha-galactosidase, beta-galactosidase, alpha-glucosidase, beta-glucosidase, mannosidase, isomerase, invertase, transferase, ribonuclease, chitinase, mutanase and deoxyribonuclease.

108. Dimorphic fungal cell according to claim 83 comprising i) a first nucleotide sequence comprising the coding sequence of SEQ ID NO:11, or a fragment thereof encoding a regulator of morphology of said dimorphic fungal cell, said first sequence being operably linked to a second nucleotide sequence comprising SEQ ID NO:9, or a fragment thereof capable of directing gene expression in said dimorphic fungal cell, said cell further comprising ii) a first nucleotide sequence comprising the coding sequence of SEQ ID NO:1, or a fragment thereof encoding a regulator of morphology of said dimorphic fungal cell, said first sequence being operably linked to a second nucleotide sequence comprising SEQ ID NO:14, or a fragment thereof capable of directing gene expression in said dimorphic fungal cell.

109. Dimorphic fungal cell according to claim 83 comprising i) a first nucleotide sequence comprising the coding sequence of SEQ ID NO:11, or a fragment thereof encoding a regulator of morphology of said dimorphic fungal cell, said first sequence being operably linked to a second nucleotide sequence comprising SEQ ID NO:13, or a fragment thereof capable of directing gene expression in said dimorphic fungal cell, said cell further comprising ii) a first nucleotide sequence comprising the coding sequence of SEQ ID NO:1, or a fragment thereof encoding a regulator of morphology of said dimorphic fungal cell, said first sequence being operably linked to a second nucleotide sequence comprising SEQ ID NO:14, or a fragment thereof capable of directing gene expression in said dimorphic fungal cell.

110. Dimorphic fungal cell according to claim 83 comprising i) a first nucleotide sequence comprising the coding sequence of SEQ ID NO:11, or a fragment thereof encoding a regulator of morphology of said dimorphic fungal cell, said first sequence being operably linked to a second nucleotide sequence comprising SEQ ID NO:13, or a fragment thereof capable of directing gene expression in said dimorphic fungal cell, said cell further comprising ii) a first nucleotide sequence comprising the coding sequence of SEQ ID NO:1, or a fragment thereof encoding a regulator of morphology of said dimorphic fungal cell, said first sequence being operably linked to a second nucleotide sequence comprising SEQ ID NO:10, or a fragment thereof capable of directing gene expression in said dimorphic fungal cell.

111. Dimorphic fungal cell according to claim 83 comprising i) a first nucleotide sequence comprising the coding sequence of SEQ ID NO:11, or a fragment thereof encoding a regulator of morphology of said dimorphic fungal cell, said first sequence being operably linked to a second nucleotide sequence comprising SEQ ID NO:9, or a fragment thereof capable of directing gene expression in said dimorphic fungal cell, said cell further comprising ii) a first nucleotide sequence comprising the coding sequence of SEQ ID NO:1, or a fragment thereof encoding a regulator of morphology of said dimorphic fungal cell, said first sequence being operably linked to a second nucleotide sequence comprising SEQ ID NO:10, or a fragment thereof capable of directing gene expression in said dimorphic fungal cell.

112. Dimorphic fungal cell according to claim 83 comprising i) a first nucleotide sequence comprising the coding sequence of SEQ ID NO:11, or a fragment thereof encoding a regulator of morphology of said dimorphic fungal cell, said first sequence being operably linked to a second nucleotide sequence comprising SEQ ID NO:14, or a fragment thereof capable of directing gene expression in said dimorphic fungal cell, said cell further comprising ii) a first nucleotide sequence comprising the coding sequence of SEQ ID NO:1, or a fragment thereof encoding a regulator of morphology of said dimorphic fungal cell, said first sequence being operably linked to a second nucleotide sequence comprising SEQ ID NO:10, or a fragment thereof capable of directing gene expression in said dimorphic fungal cell.

113. Dimorphic fungal cell according to claim 83, wherein said cell comprises a replicon harbouring a selectable marker conferring resistance against an antibiotic.

114. Dimorphic fungal cell according to claim 113, wherein said antibiotic is geneticin.

115. Dimorphic fungal cell according to claim 113, wherein said replicon is capable of undergoing extrachromosomal replication in a bacterial cell.

116. Dimorphic fungal cell according to claim 113, wherein said replicon comprises SEQ ID NO:9, or a fragment thereof capable of directing gene expression.

117. Dimorphic fungal cell according to claim 113, wherein said replicon can be maintained at about 0.5 copies per nucleus at a first predetermined concentration of antibiotic, and maintained at about 2 to 50 copies per nucleus at a second and higher predetermined concentration of antibiotica.

118. Method for constructing a recombinant fungal cell according to claim 82, or a recombinant dimorphic fungal cell according to claim 83, said method comprising the step of transforming or transfecting a polynucleotide according to any of claims 1 to 74, or the vector of any of claims 80 and 81, into a fungal cell or a dimorphic fungal cell.

119. A method of claim 118 and comprising the further step of transforming or transfecting said recombinant fungal cell or said recombinant dimorphic fungal cell with a further polynucleotide comprising
i) at least one nucleotide sequence encoding a gene product, and operably linked thereto, and
ii) at least one further nucleotide sequence comprising a further expression signal capable of directing the expression in a dimorphic fungal cell of the at least one nucleotide sequence encoding the gene product, wherein said further expression signal is regulatable, during growth of the dimorphic fungal cell, by one or more of
a) the composition of the growth medium, including at least one of carbon source, nitrogen source including amino acids or precursors thereof, oxygen content, ionic strength, including NaCl content, pH, low molecular weight compounds, cAMP, and the presence or absence of a cell constituent, or a precursor thereof,
b) the temperature of the growth medium, including any change thereof, including an upshift eliciting the expression of one or more heat shock genes,
c) the growth phase of the dimorphic fungal cell, and
d) the growth rate of the dimorphic fungal cell,
wherein the nucleotide sequence encoding the gene product and the further nucleotide sequence comprising the regulatable expression signal are not natively associated.

120. Method for regulating the morphology of a recombinant fungal cell according to claim 82, or a recombinant dimorphic fungal cell according to claim 83, said method comprising the steps of
i) cultivating said fungal cell or said dimorphic fungal cell under conditions allowing expression of said first nucleotide sequence encoding the at least one regulator of morphology, and
ii) regulating the morphology of said recombinant fungal cell or said recombinant dimorphic fungal cell.

121. Method for obtaining a predetermined dimorphic shift of a dimorphic fungal cell according to any of claims 83 or 86, said method comprising the steps of
i) cultivating said dimorphic fungal cell under conditions allowing expression of said first nucleotide sequence encoding the at least one regulator of morphology, and
ii) obtaining a predetermined dimorphic shift of said dimorphic fungal cell, wherein said dimorphic shift results from regulating the expression in said dimorphic cell of said regulator of morphology.

122. Method for increasing the filamentation of a dimorphic fungal cell according to any of claims 83 or 86, said method comprising the steps of
i) cultivating said dimorphic fungal cell under conditions allowing expression of said first nucleotide sequence encoding the at least one regulator of morphology, and
ii) increasing the filamentation of said dimorphic fungal cell, wherein said increased filamentation results from regulating the expression in said dimorphic cell of said regulator of morphology.

123. Method for increasing the secretory capacity of a dimorphic fungal cell according to any of claims 83 or 86, said method comprising the steps of
i) cultivating said dimorphic fungal cell under conditions allowing expression of said first nucleotide sequence encoding the at least one regulator of morphology, and
ii) increasing the secretory capacity of said dimorphic fungal cell, wherein said increased secretory capacity results from regulating the expression in said dimorphic cell of said regulator of morphology.

124. Method for producing a gene product in a dimorphic fungal cell according to claim 86, said method comprising the steps of
i) cultivating said dimorphic fungal cell under conditions allowing expression of said first nucleotide sequence encoding said at least one regulator of morphology, and
ii) cultivating said dimorphic fungal cell under conditions allowing expression of said nucleotide sequence encoding said gene product, and
iii) producing a gene product.


SEQUENCE LISTING (65 sequences)

SEQ ID NO: 1 - DNA, 2525 bp, Mucor circinelloides
  CDS (542)..(724): Exon of pkar
aagctttattcatttcactggtcaacgtaagtacatttctctcagtattggtcgcttttatatcatctttttggctgctttacgtgatgaacaaaacattatgctactaaacccagctcagtttgagatattcggtgaaagaaactatttccataactgaaaaagttaaaccaaaaagatatatgaaaatgatacatttacttgttcatttgagctccatattaatcctcttctcctctagttggcatgtctttttgcaagccaaagctacctatagctcaggtctattagatgtatcatcttgatcttttttgaattgaataaataaatttcttgtattttaaaatgtaacactttaatgcctaatttctgcgtgcaatgtcgtttttttttctgtgataaccctgaactgctcaaatgctttcatgatgtcatctcataatctgttgggttacatccaatactgttaaattgtatgtgttgatcttgagtataagggatcgatcatttgattgtctttttctctcctattttcattaaaatgatcactgacgaacatccgtttgaatttgcgcctcagcaagatgaatacacgcagctgttgacagagttacataacgaatactgcgctgagcaaccactagatgtgcttcagttctgctccaactttttcattcgcaaactcgaagagcagcgcttggagcatagaaacaaccaccattcccgtaacaacttgtttgatagtaaagtgtctctgccacgagcctagtgatggatgctaacgtttttccttagccnaatgataccagtaatgatttacatcctttgtgtgagcaaccacaagaagacttttcacaacagcaaggcatccagtgggaaaccacgcatatgggccatcccaacgaccacggtgctcttcatgatgatgatgatgatccgttggaagacgaagacgatgaagagtttgacaaattttcaactgaacctttgccctcgctgcctcccacaaactataaccgtggccgccgcacatctgttaagtgcagagagcatggcacccagcgccaaccaagactttgtcaaggtcatcatccccaaatctcaggcacaagcgagcgcatcaaagtctccatcagcaacaactttttgtttcgcaacctggacgaagagcagtacctggatgtggtgaatgccatgtctgaaaagcgcgtcgtcaagggcaccacagtgatcgagcaaggcagtgtgggtgatttcttctacgtcgtcgagtcgggtactttggattgttttattgggcaaaacaaggttaccaactatgaggcaggtggtagcttcggtgaattagccttaatgtacaacgcccctcgtgctgctactattattacaacatcagactctgtgctttgggctctggacagaaacacttcggcaccatccttgatggagaacacctcacgcaaaagacgcatgtatgaatacttcttatcagaagtcgtcttgttaaaatccctggaatcatatgaacagcataaaattgcggatgccctcgaatcagtttattttgaagatggacaggaggttgtgaagcagggtgatgtcggagatcagttctacatcattgaatccggtgaagccatcgtcctgaaggaagagaacggcgtccagcaacaggtgaaccagcttgagcgaggatcctactttggaggtaagatggagcttgttggggttggtgatgtgtcgctaaccactgtgtgatagaactggccctgttaaacgatgctcctcgagctgcaaccgtagttgctcacggcagactcaagtgcgctacactgggcaaaaaggcattcactcgtcttcttggccctgttttggacatcttgaagcgtaattcagaaaactatcatgctgtcattaaccagcaatcataatcgcaccaaaaagttacactagatttcaaataaaaaccatggatactttccgatctgatgttgacttgactgtaacaaagcgacaggaaaaagaaacttgatttgcttcctgaccaacaatgcagccaatctccttaaacaagatgctctctatttcggcctgaaaatataacctccttgatttcgtattttgktgttgtgcttttttccctctctctctcttcttcttttcactcttgttataaaaaaaatatgacgggtatgattcacagtatggagagcaacccttgatgagcctccacctcaaagcgccagcggcctcttctaatctgcctggcacaggtattgccaatctaccaaatcaaagacacaagattgttgccaaaaatggcgccaatttcaccatcatggtttgtggtaagacatatgtatacttgcaagtgaaaggaccaggtaactgaattttgcttaggtgaatcgggtgtcggaaaaacaacctttgtaaacacactgttcacatccaccatcaaggagccaaagaacctgacaaagagacatctcaagacaccttccaagacggtgcaaatccagatc

SEQ ID NO: 2 - protein, 421 aa, Mucor circinelloides
MITDEHPFEFAPQQDEYTQLLTELHNEYCAEQPLDVLQFCSNFFIRKLEEQRLEHRNNHHSPNDTSNDLHPLCEQPQEDFSQQQGIQWETTHMGHPNDHGALHDDDDDPLEDEDDEEFDKFSTEPLPSLPPTNYNRGRRTSVKCREHGTQRQPRLCQGHHPQISGTSERIKVSISNNFLFRNLDEEQYLDVVNAMSEKRVVKGTTVIEQGSVGDFFYVVESGTLDCFIGQNKVTNYEAGGSFGELALMYNAPRAATIITTSDSVLWALDRNTSAPSLMENTSRKRRMYEYFLSEVVLLKSLESYEQHKIADALESVYFEDGQEVVKQGDVGDQFYIIESGEAIVLKEENGVQQQVNQLERGSYFGELALLNDAPRAATVVAHGRLKCATLGKKAFTRLLGPVLDILKRNSENYHAVINQQS

SEQ ID NO: 3 - DNA, 634 bp, Mucor circinelloides
  CDS (1)..(273): Exon of ste20
tccgccagcaacaggatgccaaaaagactggtcgaaaccgcagagccatcgccttcatctcaaacaarnatggacgattttgaaatcaaacagccaataggtaacagatggacggcatctgcatgtactgttactgatagacacctgcttcaaggctacggatcatctgccatggtttatagcgcagtgtatatacctcacaacaaacgggtcgccatcaaggtgattgatctggacatgtttgagcgcaaccaaatagacgagctgagggtagtacatggcagcacacactaggattcccttcttattgacaaaacgtatatatngagagagacagccttgatggctctgtccaagcatccacatgtgttgcgagtctacggctcatttgtccacggatccaagctgtacattgtcactccttatatggcagtaggatcctgtctcgatatcatgaagttgagtttccccgacggcctagacgagattagcattgctactatcctaaaacaggcactggaaggactagcctatttgcacaaaaatggccacatccatcgagacgtaaaggcaggcaacctgctgatggatgaggacggctctgtgctgctggcggatggtgtgctcaccaaag

SEQ ID NO: 4 - protein, 193 aa, Mucor circinelloides
  misc_feature (23)..(23): The ′Xaa′ at location 23 stands for Arg, Ser, Lys, or Asn.
SASNRMPKRLVETAEPSPSSQTXMDDFEIKQPIGNRWTASACTVTDRHLLQGYGSSAMVYSAVYIPHNKRVAIKVIDLDMFERNQIDELRVRETALMALSKHPHVLRVYGSFVHGSKLYIVTPYMAVGSCLDIMKLSFPDGLDEISIATILKQALEGLAYLHKNGHIHRDVKAGNLLMDEDGSVLLADGVLTK

SEQ ID NO: 5 - DNA, 541 bp, Mucor circinelloides
  CDS (2)..(133): Exon i mkp1
ttacatagttcaggagattatggaggctgatcttcaccagattattcgctccggccagccgttgacggatgctcatttccaatactttgtctaccaaatctgcagaggactaaagtacattcacagtgccaatgtaagcatatatagacgatttgacaacatgcgtattaatgtgctttgctctcaaaggtgttgcatcgagatctcaagccaggtaaattacgaataaacggcataacacagatcacgtcgtgatatttatcatgtgataatttataaacaggcaacctccttgtcaacgctgattgcgaattaaaggtaaggaaacacagggtgcagacaattcgtacatgtattaaatcgagggaaccaaagatttgtgatttcggcttggctcgtggctattctgagaacgacgaacacaatgtgggcttcatgaccgaatatgtaagttatctgatgcttgagtgtgaggacgtggtgtaacagtgtgtttatttgaaaggttgcaacaagatggtaccgcgcccctgaaataatg

SEQ ID NO: 6 - protein, 103 aa, Mucor circinelloides
YIVQEIMEADLHQIIRSGQPLTDAHFQYFVYQICRGLKYIHSANVLHRDLKPGKLRINGITQITEPKICDFGLARGYSENDEHNVGFMTEYVATRWYRAPEIM

SEQ ID NO: 7 - DNA, 384 bp, Mucor circinelloides
  CDS (1)..(384)
aagttttttcttgctacggctcctgtgaattgggagcacaacaaaccgttaaagcgctttgcattaccaggcggttcagcagcagcagcacccggcggacgatcgcccaacggcagcggcgagagcatttcgtgcgtcttgtggaacgacctgttcttcatcacaggcaccgacattgtgcgctcgctgacctttcgcttccatgcgtttggccgacccgttacgaacgcaaagaagtttgaagagggcatattttctgatttgcgcaacttaaaaccaggtcatgatgctcggttggaggaacccaaatctgaattgctggacatgctctacaagaacaattgcatccgcacacaaaaaaaacaaaaagtatttttctggttt

SEQ ID NO: 8 - protein, 128 aa, Mucor circinelloides
KFFLATAPVNWEHNKPLKRFALPGGSAAAAPGGRSPNGSGESISCVLWNDLFFITGTDIVRSLTFRFHAFGRPVTNAKKFEEGIFSDLRNLKPGHDARLEEPKSELLDMLYKNNCIRTQKKQKVFFWF

SEQ ID NO: 9 - DNA, 741 bp, Mucor circinelloides
aagctttcaaatgtgttggatgaacaattcatcctataatctctaatgaaatcccgaagatctacacagcatcacattcgatagatggggctgctgtttatgtgattaaaacctcactgatattatctgtttcatgtaaaaaaaaactctgttgtggtacaaacattagtgtgaaccacgcgcagccataccactagtcaaaataatgctctactgcaaaaaatgacgtttgacgaataatgcaacgtaaagatggtttagaaacccttgatatccaaattacacgtgtagcagccttcgtgggtatttttcatcacaacactactaggtagctcagggatagttcaaacgggcaatttccatcctcatcacactttattcaccaaggaaagaagtgaaatggcatcttctatcgttcaacatctacagggacatctgtgagatacatctgattgctcgacaagcggacaatagatgacacgttatcaatgctatcactctaaaatgtcatgtctgactgagtccattgcaatcatcactccatccgacatcaggtcacaatttatgcttctattttccaatggatccgaatccgattcaaacaagattaattctccctcaaaatacccatgaagtgtgagacattgcgaaatgttatataaacccaatgcatttctcgtctttcagggtttttttcttcttcttcatactatatctctatatattttataaattctaaca

SEQ ID NO: 10 - DNA, 755 bp, Mucor circinelloides
cgttaattaagatatgattaggttgctaatgaatcatatcgattatcttcatcactcgattagactgccgatctttctttgcttctgttcgtttttccatgtcatctggtggtacctgggtgtgattgagcgaaaacttccatggaggtaactcccgaaactttgtttttctcactgggcttgataaaatgaaaaaactaaaaatctcacgaacatggtacaaggggaaaccttgaaattagtagcggatgatgccagcataacgatgatttctctctctctgctctactcttttatgttgtggtgattctcttcacagagcagcactactgtcaacatggagcgatattctccaatttctccaaatgtcggtatttcataaattgagatgcctttcaaatgcctttcagatgcctttccaaggcacttgctaaaataatgcatttgctggcatacaaacaataactaattctccgggaattgccgggcaaatcaccttgtgtgcagtgattagtatatcgaaaggcggggatatctagaactttgtttgtgtggtaacattaaggtttagaagcctttttttatagcgtcctaccatgacttcatgtggaggatccaatcaagtctttatttatacctttgacagggttaaactaaaaacaatttagaaaaaagaaaaactataaaagccatccaacattccagcaatgcctgcctctcttcttctcttcaacactctattctgttaaacaaca

SEQ ID NO: 11 - DNA, 2578 bp, Mucor circinelloides
  CDS (534)..(1028)
  Intron (1029)..(1085)
  CDS (1086)..(1307)
  Intron (1308)..(1370)
  CDS (1371)..(2468)
tcgaaatcaaaggtctgctactagtctaataccacaggaagcagatattggtatttgaaactgccacgttattgaaatgcctctgatcgtatgactagctggcccaatgaacattacattggccccaacagccaatcaaagacgtcccaatttaaaggggatgttggcatctaatgttgatcgcagatagacacacctaaaattatgcactgttttgggttacacattgattttaggtaaccacaccatctagaattcaggacatgtagaagccggtatatgagatggaaggtacattgtttacaagtactagcgtcaataaagtatcaaatagattcagtgagtagtctgctatcactctatactagcgagcacagtcaatcggccgataagaaataggaacagaaatatacccccaacatcagtggtcttcaacggaatctcaaacatgaaactgttaaatatgagatggatcttgcctattcttctctcttgctcattcttctcatcatcgcagaatacacatacgtaatatggctgatttcacagattctctcatcaagaacattggcgttcactcatcatctcctgtcatgacatctgtcaatatgggtcaattgggtgaaaagcttcgtcaagctcgtacaacaacacttgcttccttatctcaagctctttcaaagaagcccgaagctgctgctgctgctgccactgcccccaacgctgttaatgaaagtaccaccacacccaccacaatgcaactccctgcttcggaaaaagccactagtcaattggagatcaatgtggttgaagctcgtaatttgaccattgctgatgcgcgcaaagccgacacctactgtattgttcattacgaaggcaacaccacatcaacgcttgataaagtagatgatggcatcttgcccagcacgcctctggtgattaaatctcaagtcgctagcggtgcattcaaggcatttgaaatcatgatgagcgctagttctcccaagtggatgcatcgtgtcaacttgtaagttgctatccagaatatgtcaaaaagggctctgcgctaaccatgttactatagtgatgtaactgctggtaacaaggagatcactgtgtttgtctatgatcgtggtaacaaattgcccaatggtgaagatcgcttcttgggcatgtctagcattgttcccaacttggtcaacaagaagacggtcgagctgatctttcctcttcacggccgtcctgacgatgatcaagaagttactggtgatgtccgtcttcaagttacttttatcgaccctaaaaaggtaattttatatgagtatgattcttgacagctgatgtctgacacttctaaaaccctattcaaggctaatcttaagccagaggatttccgcattgtgcgtatgattggtcaaggctcagtgggtaaggtgtatgaggtgatcaagcgtgattctggccgtacctatgccatgaaggtgctctctaagcgtctcttgctcgccgagaatgaagtcgatactgccttcaacgagcgcaatgtgctggttcagtctctctcaagccctttcattgccaatctcaagtacagtttccaaacaacaaaccatctcttcttggttatggattactttccgggtggcgaattgtttgatttcctggagcgtgagcgttgtttgagcgagaagcgttgccaattctttgctgccgagattgtgtgtgcctttgacaacatccatgctcgcaacattgtctatcgtaacctgaagccagagagcatcttgctggatgcacatggacacattgccttgacagatttcggcttatgcaagcaattgaagaacaagatggatttgattcaaggtgtgcctcaagtcattacacaagaatacctcgcccctgaaatggtaatgcaaaagccctatggcatggctgccgactggtggagtctcggtgttttgatgtttgagctgttgactggatctcctcctttccattctgttgaacaaggtgaattgtttagacaaatcctggaagctcccattaaattccctgctgggggctgcattacagaggaagccaaggatttcatctgccaactgctggagcgtgatcctgccaagcgtctgggctcccatggtgatgttgctcaggtcaaagcacatccattctttaaggatctcaactgggatgtcgtttacaagaagcaaatgcagcttccctttgtgcccgaggtagaagagcagctccgcgaagaagccattgctgctgctgctgccattagcattcctgtgaccaacagcaagaccgagtctaccaatgccaatgtgatgcctgtggctgatcaatccaaattcaagggatttagctatattcgtgaagatgtcatggcaaagaagggcgagcatcgtctgggtgtcaatcctgaggatgaagatcccgaagttgatttctggtttagacagtaaaaatcgtccatctatccttacattttgtacatatatattaatcaagacccccctcctcattcaataaagcacatatttgttcatataccaaaaaaaaaaaaaaaaaa

SEQ ID NO: 12 - protein, 605 aa, Mucor circinelloides
MADFTDSLIKNIGVHSSSPVMTSVNMGQLGEKLRQARTTTLASLSQALSKKPEAAAAAATAPNAVNESTTTPTTMQLPASEKATSQLEINVVEARNLTIADARKADTYCIVHYEGNTTSTLDKVDDGILPSTPLVIKSQVASGAFKAFEIMMSASSPKWMHRVNLDVTAGNKEITVFVYDRGNKLPNGEDRFLGMSSIVPNLVNKKTVELIFPLHGRPDDDQEVTGDVRLQVTFIDPKKANLKPEDFRIVRMIGQGSVGKVYEVIKRDSGRTYAMKVLSKRLLLAENEVDTAFNERNVLVQSLSSPFIANLKYSFQTTNHLFLVMDYFPGGELFDFLERERCLSEKRCQFFAAEIVCAFDNIHARNIVYRNLKPESILLDAHGHIALTDFGLCKQLKNKMDLIQGVPQVITQEYLAPEMVMQKPYGMAADWWSLGVLMFELLTGSPPFHSVEQGELFRQILEAPIKFPAGGCITEEAKDFICQLLERDPAKRLGSHGDVAQVKAHPFFKDLNWDVVYKKQMQLPFVPEVEEQLREEAIAAAAAISIPVTNSKTESTNANVMPVADQSKFKGFSYIREDVMAKKGEHRLGVNPEDEDPEVDFWFRQ

SEQ ID NO: 13 - DNA, 927 bp, Mucor circinelloides
tctttatcaccaaatcagcacgagcaatttgctaatctaaccgtgcaagacttgtcattctatgaccaacatccacatttgcaacaacaacaacaacaacaacagcaacaccaccaccaccaacaagagccattaacgtggacagatttgcccttttgtaagtactcaaattagtcaagtgatagactcacacactcacactcacacaaacctctagatgaagatccctctctcatgatgacaccaactacaccatctatatttacagctaataacaacaacccctatgatatcccttcttctgcctcaaatgctacacacaccgcatctactacacatactactaatacacaaatcatatctgccgaagcactgcaaattggtacctggaagagaatgacatttgaacccaatgacctctcatgccagttcgatagagacagcaaactcttcagctggtgcatccaagacggtatttccaagttcaaaatggaattcccacaagaatttgtgcaatccatcaagctatcacccttaacaagtcgacctggctgggcagattggagatgaatgtactatctactcaacacatcttgttctacatggagacgccgcaacaaagctggattcaatgccgcgactacactgaagacaagcaggcttccatcatcagcctgcaccaactagacggccctgcacttgcattaaaggcagaactagaatccctctctaaggaaaacgactatctagctaccatcattcattaatttgcatatcattgattggtgcgcctgattaaaattgtgtaatataaaataccatgttgacctctccccctccatttttctcttcttcttcttcttcaacctttgttgcttattctcccttaacttttgaataaatcaactttctaaacaccctataaaa

SEQ ID NO: 14 - DNA, 419 bp, Mucor circinelloides
gatctctcggtttttttttctcttgcaacatgtggtacatgcatttccagcttggatggctcactcattccaaagaaagtattccttggatctcaaatgcaatccaatggaaaacagatgcttgggtcgtcttggtggcataaattggaaaaactgggttttccgttcataaggtcccattttccgtggaaagtctaaaatcgactgacttttttccaatgaggaaagcctggaggaggtcgacttgtatcacaacaaggttgcttatgaaatcaacagagtcacatcccgtctaaaacccagtttggatccgttttcttcgcttctatctgtgggtgcgaggatttggtataaaaaggactagattctccacaacaatttccattttttccctcattatcattcaataatactgtaaa

SEQ ID NO: 15 - DNA, 24 bp, Artificial Sequence
  Oligonucleotide primer
ggngaytayttytaygtngtngar

SEQ ID NO: 16 - DNA, 24 bp, Artificial Sequence
  Oligonucleotide primer
raangtnacnckrtcnarngccca

SEQ ID NO: 17 - DNA, 36 bp, Artificial Sequence
  oligonucleotide primer
actgcctcgagatgatcactgacgaacatccgtttg

SEQ ID NO: 18 - DNA, 42 bp, Artificial Sequence
  oligonucleotide primer
acgctagcggccgccgcctgcgctttgaggtggaggctcatc

SEQ ID NO: 19 - DNA, 21 bp, Artificial Sequence
  oligonucleotide primer
ggnaarggnacnttyggncar

SEQ ID NO: 20 - DNA, 24 bp, Artificial Sequence
  oligonucleotide primer
rttytcnggyttnarrtcnckrta

SEQ ID NO: 21 - DNA, 33 bp, Artificial Sequence
  oligonucleotide primer
raaccaraaraanacyttytgyttyttytgngt

SEQ ID NO: 22 - DNA, 21 bp, Artificial Sequence
  oligonucleotide primer
tayathgtncargarathatg

SEQ ID NO: 23 - DNA, 24 bp, Artificial Sequence
  oligonucleotide primer
catdatytcnggngcnckrtacca

SEQ ID NO: 24 - DNA, 2471 bp, Mucor circinelloides
  CDS (742)..(1038)
  Intron (1039)..(1095)
  CDS (1096)..(1668)
  Intron (1669)..(1724)
  CDS (1725)..(1865)
  misc_feature (2059)..(2059): n is a, c, g or t
aagctttcaaatgtgttggatgaacaattcatcctataatctctaatgaaatcccgaagatctacacagcatcacattcgatagatggggctgctgtttatgtgattaaaacctcactgatattatctgtttcatgtaaaaaaaaactctgttgtggtacaaacattagtgtgaaccacgcgcagccataccactagtcaaaataatgctctactgcaaaaaatgacgtttgacgaataatgcaacgtaaagatggtttagaaacccttgatatccaaattacacgtgtagcagccttcgtgggtatttttcatcacaacactactaggtagctcagggatagttcaaacgggcaatttccatcctcatcacactttattcaccaaggaaagaagtgaaatggcatcttctatcgttcaacatctacagggacatctgtgagatacatctgattgctcgacaagcggacaatagatgacacgttatcaatgctatcactctaaaatgtcatgtctgactgagtccattgcaatcatcactccatccgacatcaggtcacaatttatgcttctattttccaatggatccgaatccgattcaaacaagattaattctccctcaaaatacccatgaagtgtgagacattgcgaaatgttatataaacccaatgcatttctcgtctttcagggtttttttcttcttcttcatactatatctctatatattttataaattctaacaatggttgttcaagtcggtattaacggtttcggtcgtattggtcgtattgtccttcgtgctactgagtccaacaaggatgtccaagttgttgctatcaacgatcccttcattcctctcgactatatggtctacatgttgaagtacgatactgttcacggtcgtttcgatggttccgtcgaggccaaggatggtaagctcgttgtcaacggtcatgctatcgccgtctctgctgagcgcgatcctacctctattccttggggttccgctggtgctgactacgttgtcgagtccactgggtaaatatactgaaatgcattatatctcgaatatctaatctaacattgacgtaatagtgtcttcactaccactgaagctgcctctgctcatcttaagggtggtgccaagaaggtcatcatctctgctccctctgctgatgcccccatgttcgtctgtggtgtcaacctcgaagcttacaagtctgaatacaaggttatctccaacgcctcttgtaccaccaactgtttggctcccctcgccaaggtcattaacgataactttggtatcgctgatggtttgatgaccactgtccacgccaccactgccacccaaaagactgtcgatggtccctctcacaaggattggagaggtggtcgtgccgctgctgccaacatcatcccctcttccactggtgctgccaaggctgtcggtaaggtcattcccgctctcaacggtaagctcactggtatggctttccgtgtccccacccccgatgtctctgtcgttgatttgaccgtcaacctctccaagggtgcttcttatgatgaaatcaagcaagccatcaagaaggcctctgaaactaccatgaagggtgtcctcggttacacttctgatgctgttgtcagcagtgatttcgtgggtgaagtttggtaagaaacgttattatttcatcgtttgaatagtttactaacattgaaaatcatagttcttccgtatttgacgctgctgccggtatccaattgacccccacttttgttaagcttatcgcttggtatgacaatgagtatggttactctaaccgtgtcgttgacctcctcgttcatgccgctaaggtcgatggtgctctctaaatcgtaaatcatttctagtcattgcatttcatacacacatctgttacataaataaacttcatgtaaaaagtcggtcataagatcgttttttgttaattagcttatattaatttctgttccaaccctctgatatgtaaaatgttgacgaattgcaagtattttgacaggcagaatgacagcatatatttgangcctgtgvacaatctgtgttacataagattcctggtaaaggatggatgatattatattttacagttataagagccggtattggcacacgaaggaagccttgcagcgagaaggacgacgctcttttttataggctcatcactcaatgagagttgcaggaagcactattttgtaaatgcctgaaatacagagaccctctggactattattctcaagaagcactttaacaagaaaaatatagttcttttgctaatttcaagaccttaatcatatatnncgctttcatttttatttcatggtttcattcaatttatagatgtattactacactactgattgctgttactgttactatcgccctggccattgttgttgttgttgtcgctgccatcgcatcgccgttattgtcatcgc

SEQ ID NO: 25 - protein, 337 aa, Mucor circinelloides
MVVQVGINGFGRIGRIVLRATESNKDVQVVAINDPFIPLDYMVYMLKYDTVHGRFDGSVEAKDGKLVVNGHAIAVSAERDPTSIPWGSAGADYVVESTGVFTTTEAASAHLKGGAKKVIISAPSADAPMFVCGVNLEAYKSEYKVISNASCTTNCLAPLAKVINDNFGIADGLMTTVHATTATQKTVDGPSHKDWRGGRAAAANIIPSSTGAAKAVGKVIPALNGKLTGMAFRVPTPDVSVVDLTVNLSKGASYDEIKQAIKKASETTMKGVLGYTSDAVVSSDFVGEVWSSVFDAAAGIQLTPTFVKLIAWYDNEYGYSNRVVDLLVHAAKVDGAL

SEQ ID NO: 26 - DNA, 33 bp, Artificial Sequence
  oligonucleotide primer
aarttyttyytngcnacngcnccngtnaaytgg

SEQ ID NO: 27 - DNA, 23 bp, Artificial Sequence
  oligonucleotide primer
ccnggnmgngtnaayytnathgg

SEQ ID NO: 28 - DNA, 20 bp, Artificial Sequence
  oligonucleotide primer
ccnccccanccngcnccngt

SEQ ID NO: 29 - DNA, 23 bp, Artificial Sequence
  oligonucleotide primer
garcayggnathcarccngaygg

SEQ ID NO: 30 - DNA, 21 bp, Artificial Sequence
  oligonucleotide primer
catnccytcnccnacrtacca

SEQ ID NO: 31 - DNA, 22 bp, Artificial Sequence
  oligonucleotide primer
catccttgttggactcagtagc

SEQ ID NO: 32 - DNA, 22 bp, Artificial Sequence
  oligonucleotide primer
cttcagggttagagagagaagc

SEQ ID NO: 33 - DNA, 21 bp, Artificial Sequence
  oligonucleotide primer
ccttggggttttcgagggagg

SEQ ID NO: 34 - DNA, 36 bp, Artificial Sequence
  oliognucleotide primer
actgcggagctcattatgatcactgacgaacatccg

SEQ ID NO: 35 - DNA, 29 bp, Artificial Sequence
  oligonucleotide primer
gcgcatgcttatgattgctggttaatgac

SEQ ID NO: 36 - protein, 427 aa, Mucor circinelloides
MITDEHPFEFAPQQDEYTQLLTELHNEYCAEQPLDVLQFCSNFFIRKLEEQRLEHRNNHHSRNNLFDTNDTSNDLHPLCEQPQEDFSQQQGIQWETTHMGHPNDHGALHDDDDDPLEDEDDEEFDKFSTEPLPSLPPTNYNRGRRTSVKCREHGTQRQPRLCQGHHPQISGTSERIKVSISNNFLFRNLDEEQYLDVVNAMSEKRVVKGTTVIEQGSVGDFFYVVESGTLDCFIGQNKVTNYEAGGSFGELALMYNAPRAATIITTSDSVLWALDRNTSAPSLMENTSRKRRMYEYFLSEVVLLKSLESYEQHKIADALESVYFEDGQEVVKQGDVGDQFYIIESGEAIVLKEENGVQQQVNQLERGSYFGELALLNDAPRAATVVAHGRLKCATLGKKAFTRLLGPVLDILKRNSENYHAVINQQS

SEQ ID NO: 37 - protein, 411 aa, Aspergillus niger
MAESAFPSAQQPLRVGTKDDKAAAFQKISEEDEYEVTSPTDPTFRSANAAAASSSTGSPFFGGSYGENSGPIRFNRSPFDNGPREEDEEGADEFPPEDIRPTGAANQGFPNNYALGRRTSVSAESLNPTSAGSDSWTPPYHEKTEEQLSRLKTAVSSNFLFSHLDDDQFKSVLDALVEKPIPAKGIKVISQGDAGDYFYIVENGHFDFMIHPSGSVQPGPDGMGNKVGSVGPGGSFGELALMYNAPRAATVVSVDPKSTLWALDRITFRRILMDSAFQRRRMYEAFLEEVPLLSSLKPYERAKIADALDAIKYPAGSTIIAEGDPGDAFYLLESGEADAFKNGVEGPVKSYKRGDYFGELALLDDKPRAASIVAKTDVKVAKLGRDGFKRLLGPVEDIMRRAEYESNPVPA

SEQ ID NO: 38 - protein, 403 aa, Blastocadiella emersonii
MADYTIPSELPPILKDLSREVLRHQPADLVQFCHDYFAKLLAQQRKVLMDSADPATKATIASTAGPAVDADEAARANSYAYSTDDGFGTEDDDDDDDDEDDEAAIPPPVVNRGRRTSVSAESMAPTAHDVDAVKTVIPKSDEQRARIQASIGNNFLFRNLDEDQYTDVVNAMAEKKVAAGEVVIRQGGVGDYFYVVETGALDVFVNRNGNGDVKVTDYSAGGSFGELALMYNAPRAATVVATAESVLWALDRVTFRRILMDHTSRKRRMYEAFLEEVPLLSSLEPYERHKIADALESVAYADGDVVIRQGDVGENFYIIEAGDAEVIKIDENGEEHHFRPLHKGNYFGELALLSDKPRVATIRAKGKLKCAKLGKKAFTRLLGPLADIMQRNTQDYEKYPGEH

SEQ ID NO: 39 - protein, 459 aa, Candida albicans
MSNPQQQFISDELSQLQKEIISKNPQDVLQFCANYFNTKLQAQRSELWSQQAKAEAAGIDLFPSVDHVNVNSSGVSIVNDRQPSFKSPFGVNDPHSNHDEDPHAKDTKTDTAAAAVGGGIFKSNFDVKKSASNPPTKEVDPDDPSKPSSSSQPNQQSASASSKTPSSKIPVAFNANRRTSVSAEALNPAKLKLDSWKPPVNNLSITEEETLANNLKNNFLFKQLDANSKKTVIAALQQKSFAKDTVIIQQGDEGDFFYIIETGTVDFYVNDAKVSSSSEGSSFGELALMYNSPRAATAVAATDVVCWALDRLTFRRILLEGTFNKRLMYEDFLKDIEVLKSLSDHARSKLADALSTEMYHKGDKIVTEGEQGENFYLIESGNCQVYNEKLGNIKQLTKGDYFGELALIKDLPRQATVEALDNVIVATLGKSGFQRLLGPVVEVLKEQDPTKSQDPTAGH

SEQ ID NO: 40 - protein, 415 aa, Saccharomyces cerevisiae
VSSLPKESQAELQLFQNEINAANPSDFLQFSANYFNKRLEQQRAFLKAREPEFKAKNIVLFPEPEESFSRPQSAQSQSRSRSSVMFKSPFVNEDPHSNVFKSGFNLDPHEQDTHQQAQEEQQHTREKTSTPPLPMHFNAQRRTSVSGETLQPNNFDDWTPDHYKEKSEQQLQRLEKSIRNNFLFNKLDSDSKRLVINCLEEKSVPKGATIIKQGDQGDYFYVVEKGTVDFYVNDNKVNSSGPGSSFGELALMYNSPRAATVVATSDCLLWALDRLTFRKILLGSSFKKRLMYDDLLKSMPVLKSLTTYDRAKLADALDTKIYQPGETIIREGDQGENFYLIEYGAVDVSKKGQGVINKLKDHDYFGEVALLNDLPRQATVTATKRTKVATLGKSGFQRLLGPAVDVLKLNDPTRH

SEQ ID NO: 41 - protein, 412 aa, Schizosaccharomyces pombe
MSFEEVYEELKALVDEQNPSDVLQFCYDFFGEKLKAERSVFRRGDTITESFSDGDESDFLSELNDMVAGPEAIGPDAKYVPELGGLKEMNVSYPQNYNLLRRQSVSTESMNPSAFALETKRTFPPKDPEDLKRLKRSVAGNFLFKNLDEEHYNEVLNAMTEKRIGEAGVAVIVQGAVGDYFYIVEQGEFDVYKRPELNITPEEVLSSGYGNYITTISPGEYFGELALMYNAPRAASVVSKTPNNVIYALDRTSFRRIVFENAYRQRMLYESLLEEVPILSSLDKYQRQKIADALQTVVYQAGSIVIRQGDIGNQFYLIEDGEAEVVKNGKGVVVTLTKGDYFGELALIHETVRNATVQAKTRLKLATFDKPTFNRLLGNAIDLMRNQPRARMGMDNEYGDQSLHRSPPSTKA

SEQ ID NO: 42 - protein, 248 aa, Mucor rouxii
MDEEHYQDIVNAMIEKPVRKGETIIEQGAVGDYFYVVASGTFDCYIKKPGQEKPLKVTSYERGGSFGELALMYNAPRAATVTSTSESVLWALDRVTFRTILMENTALKRRVYESFLEEVALLISLEPYERHKIADSLETIFFNDNGHVISQGDIGDQFYIIESGSAIVYKTDSNGDQQMVNQLERGAYFGELALLNDCPRAATVIAKGTLRCVTLGKKAFTRLLGPVHEILKRNAENYQAILSQQQQY

SEQ ID NO: 43 - protein, 605 aa, Mucor circinelloides
MADFTDSLIKNIGVHSSSPVMTSVNMGQLGEKLRQARTTTLASLSQALSKKPEAAAAAATAPNAVNESTTTPTTMQLPASEKATSQLEINVVEARNLTIADARKADTYCIVHYEGNTTSTLDKVDDGILPSTPLVIKSQVASGAFKAFEIMMSASSPKWMHRVNFDVTAGNKEITVSVYDRGNKLPNGEDRFLGMSSIVPNLVNKKTVELIFPLHGRPDDDQEVTGDVRLQVTFIDPKKANLKPEDFRIVRMIGQGSVGKVYEVIKRDSGRTYAMKVLSKRLLLAENEVDTAFNERNVLVQSLSSPFIANLKYSFQTTNHLFLVMDYFPGGELFDFLERERCLSEKRCQFFAAEIVCAFDNIHARNIVYRNLKPESILLDAHGHIALTDFGLCKQLKNKMDLIQGVPQVITQEYLAPEMVMQKPYGMAADWWSLGVLMFELLTGSPPFHSVEQGELFRQILEAPIKFPAGGCITEEAKDFICQLLERDPAKRLGSHGDVAQVKAHPFFKDLNWDVVYKKQMQLPFVPEVEEQLREEAIAAAAAISIPVTNSKTESTNANVMPVADQSKFKGFSYIREDVMAKKGEHRLGVNPEDEDPEVDFWFRQ

SEQ ID NO: 44 - protein, 480 aa, Aspergillus niger
MPSLGGLLKKRRTKDSQTLSKELEAGSAQTQTSPNAAEDHHNHNHHQHHHHLFHHHHQPQPATNSGSAANTPPQPQDSVPQQSNRSSGAEKSSDGQVASMQSAVTQASPSAHHTSGLPQPNANAASIQNIINPSQQGAMHSASSGHTQSHHAGRSDARTTKGKYSLDDFSLQRTLGTGSFGRVHLVQSKHNHRFYAVKVLKKAQVVKMKQIEHTNDERRMLNRVRHPFLITLWGTWQDSRNLYMVMDFVEGGELFSLVRKSQRFPNPVAKFYAAEVTLALEYLHTQNIIYRDLKPENLLLDRHGHLKITDFGFAKEVPDITWTLCGTPDYLAPEVVSSKGYNKSVDWWSLGILIFEMLCGFTPFWDSGSPVKIYENILRGRVKYPPYLHPDAVDLLSQLITADLTKRLGNLHGGSDDVKNHPWFAEVTWDRLARKDIDAPYVPPIRGGQGDASQYDRYPEETEQYGMAGEDPHGHLFPDF

SEQ ID NO: 45 - protein, 425 aa, Blastocadiella emersonii
MTLIDKLMEKTKKVVGSSDKDAPAPASPSSPSTAAGAGSASSTASSTTTAAASGNLSIPSPLVAGSTTSSSISHAQKMATAAHTNSDYSPSPAATPSAPLDAVAERRRRKTTLADLELRQTLGTGSFGRVHLVRLRSTGKYYAMKVLKKAEVVKHKQVEHTLNEKGILEQIDHPFLVALHSSFQDSANLYMVMEYVTGGELFTYLRRSQRFSNNVAKFYAAEVVLAFEYLHSKDIIYRDLKPENLLLDAQGHVKITDFGFAKHVPDITWTLCGTPDYLAPEIIQSRGYGRAVDWYALGVLIFEMLAGYPPFYDEDHVRMYEKILQGKVKWPSHFDPAAKDLLKRLLTTDLTKRYGNLKGGSKDIKMHKWFAGLDWTKLFNKQIPPPYTPPNRGDGDTSNFDAYPEETEPYGKVQPDPYAQLFKDF

SEQ ID NO: 46 - protein, 442 aa, Candida albicans
MVNLLKKLHITKSHQSNHSNSDSNSLNSNTSMDNHQQQQQLQQYQQQFQQPQQQLYPGEQIVHPAAAQTGQNTTNVTAVSSSNITQSATSSLHSQQLQHVDVSKSAAEEAIRRSLLPERSTVSKGKYSLTDFSIMRTLGTGSFGRVHLVRSVHNGRYYAIKVLKKHQVVKMKQVEHTNDERRMLKLVEHPFLIRMWGTFQDSKNLFMVMDYIEGGELFSLLRKSQRFPNPVAKFYAAEVTLALEYLHSHDIIYRDLKPENILLDRNGHIKITDFGFAKEVSTVTWTLCGTPDYIAPEVITTKPYNKSVDWWSLGVLIFEMLAGYTPFYDSTPMKTYEKILAGKIHYPSFFQPDVIDLLTKLITADLTRRLGNLINGPADIRNHPWFSEVVWEKLLAKDIETPYEPPITAGVGDSSLFDHYPEEQLDYGSQGEDPYASYFLDF

SEQ ID NO: 47 - protein, 380 aa, Saccharomyces cerevisiae
MEFVAERAQPVGQTIQQQNVNTYGQGVLQPHHDLQQRQQQQQQRQHQQLLTSQLPQKSLVSKGKYTLHDFQIMRTLGTGSFGRVHLVRSVHNGRYYAIKVLKKQQVVKMKQVEHTNDERRMLKLVEHPFLIRMWGTFQDARNIFMVMDYIEGGELFSLLRKSQRFPNPVAKFYAAEVILALEYLHAHNIIYRDLKPENILLDRNGHIKITDFGFAKEVQTVTWTLCGTPDYIAPEVITTKPYNKSVDWWSLGVLIYEMLAGYTPFYDTTPMKTYEKILQGKVVYPPYFHPDVVDLLSKLITADLTRRIGNLQSGSRDIKAHPWFSEVVWERLLAKDIETPYEPPITSGIGDTSLFDQYPEEQLDYGIQGDDPYAEYFQDF

SEQ ID NO: 48 - protein, 512 aa, Schizosaccharomyces pombe
MDTTAVASKGSTNVGSSTDTLSTSASLHPSMNAGSVNEYSEQQRHGTNSFNGKPSVHDSVGSDASVSNGHNNHNESSLWTSGIPKALEEATKSKKPDSLVSTSTSGCASAHSVGYQNIDNLIPSPLPESASRSSSQSSHQRHSRDGRGELGSEHGERRSAMDGLRDRHIRKVRVSQLLDLQRRRIRPADHTTKDRYGIQDFNFLQTLGTGSFGRVHLVQSNHNRLYYAIKVLEKKKIVDMKQIEHTCDERYILSRVQHPFITILWGTFQDAKNLFMVMDFAEGGELFSLLRKCHRFPEKVAKFYAAEVILALDYLHHNQIVYRDLKPENLLLDRFGHLKIVDFGFAKRVSTSNCCTLCGTPDYLAPEIISLKPYNKAADWWSLGILIFEMLAGYPPFYSENPMKLYENILEGKVNYPSYFSPASIDLLSHLLQRDITCRYGNLKDGSMDIIMHPWFRDISWDKILTRKIEVPYVPPIQAGMGDSSQFDAYADVATDYGTSEDPEFTSIFKDF

SEQ ID NO: 49 - DNA, 70 bp, Artificial Sequence
  synthetic
tttctctctttcagggtttttttcttcttcttcatactatatctctatatattttataaatctcgagatg

SEQ ID NO: 50 - protein, 125 aa, Mucor circinelloides
KFFLATAPVNWEHNKPLKRFALPGGSAAAAPGGRSPNGSGESISCVLWNDLFFITGTDIVRSLTFRFHAFGRPVTNAKKFEEGIFSDLRNLKPGHDARLEEPKSELLDMLYKNNCIRTQKKQKVF

SEQ ID NO: 51 - protein, 111 aa, Saccharomyces cerevisiae
KFFLATAPVNWQENQIIRRYYLNSGQGFVSCVFWNNLYYITGTDIVKCCLYRMQKFGREVVQKKKFEEGIFSDLRNLKCGIDATLEQPKSEFLSFLFRNMCLKTQKKQKVF

SEQ ID NO: 52 - protein, 111 aa, Candida albicans
KFFLATAPANWQENQVIRRYYLNHDEGFVSCVYWNNLYFITGTDIVRCIVYKFEHFGRKIIDRKKFEEGIFSDLRNLKCGADAILEPPRSEFLEFLFKNSCLRTQKKQKVF

SEQ ID NO: 53 - protein, 111 aa, Kluyveromyces lactis
KFFLATRPANWQENQVIRRYYLSNDEGFVSCVFWNNLYYITGTDIVRCCAYRMQKFGREIVERKKFEEGIFSDLRNLKCGIDATLEKPKSDLLAFLYKNMCLKTQKKQKVF

SEQ ID NO: 54 - protein, 110 aa, Aspergillus nidulans
KYFLLSAPVDWQPDQLIRRFLLPTGDYISCVLWSNLFHISGTDIVRCLAFRFQAFGRPVKNSKKFEEGIFSDLRNLKAGTDATLEEPKSPFLDFLYKNNCIRTQKKQKVF

SEQ ID NO: 55 - protein, 111 aa, Clavispora lusitaniae
KFFLATAPANWQENQVIRRYYLNNDEGFVSCVFWNNLYFVTGTDIVRCILYKFQHFGRTITDRKKFEEGIFSDLRNLKAGSDSVLEEPKSPFLEFLYNNSCLRTQKKQKVF

SEQ ID NO: 56 - protein, 103 aa, Mucor circinelloides; identical to SEQ ID NO: 6

SEQ ID NO: 57 - protein, 100 aa, Schizosaccharomyces pombe
YIYEELMEADLNAIIKSGQPLTDAHFQSFIYQILCGLKYIHSANVIHRDLKPGNLLVNADCELKICDFGLARGCSENPEENPGFMTEYVATRWYRAPEIM

SEQ ID NO: 58 - protein, 100 aa, Candida albicans
YLYEELMECDMHQIIRSGQPLSDQHYQSFIYQVLCGLNFIHSADVLHRDLKPGNLLVNADCELKICDFGLARGFSENPDENAGFMTEYVATRWYRAPEIM

SEQ ID NO: 59 - protein, 98 aa, Fusarium oxysporum
YLIQELMETDMHRVIRTQDLSDDHCQYFIYQTLRALKAMHSANVLHRDLKPSNLLLNANCDLKVCDFGLARSAASQEDNSGFMTEYVATRWYRAPEIM

SEQ ID NO: 60 - protein, 100 aa, Saccharomyces cerevisiae
YLYEELMECDMHQIIKSGQPLTDAHYQSFTYQILCGLKYIHSADVLHRDLKPGNLLVNADCQLKICDFGLARGYSENPVENSQFLTEYVATRWYRAPEIM

SEQ ID NO: 61 - protein, 98 aa, Candida albicans
YLIQELMETDLHRVIRTQNLSDDHIQYFIYQTLRALKAMHSANVLHRDLKPSNLLLNSNCDLKICDFGLARSIASQEDNYGFMTEYVATRWYRAPEIM

SEQ ID NO: 62 - protein, 14 aa, Artificial Sequence
  consensus sequence
ISXPXXFXHXXHVG

SEQ ID NO: 63 - protein, 16 aa, Artificial Sequence
  consensus sequence
ISXPXXXXFXHXXHVG

SEQ ID NO: 64 - protein, 99 aa, Artificial Sequence
  consensus sequence
FXXXXXXXXXXREXXXXXXXXXXXXXXXXXXXXXXXXXXXXXXXXXXXXXXXXXXXXXXXXXXXXXXXXXXXXXXXXXXXXXXXXRDXKXXXXXXXXXC

SEQ ID NO: 65 - protein, 113 aa, Artificial Sequence
  consensus sequence
FXXXXXXXXXXREXXXXXXXXXXXXXXXXXXXXXXXXXXXXXXXXXXXXXXXXXXXXXXXXXXXXXXXXXXXXXXXXXXXXXXXXXXXXXXXXXXXXXXRDXKXXXXXXXXXC